(19) United States
(12) Patent Application Publication
(10) Pub. No.: US 2021/0190760 A1
(43) Pub. Date: Jun. 24, 2021

(54) PLEURAL EFFUSION-BASED METHODS FOR ASSESSING IMMUNORESPONSIVE CELL ACTIVITY

(71) Applicant: MEMORIAL SLOAN-KETTERING CANCER CENTER, New York, NY (US)

(72) Inventor: Prasad S. Adusumilli, New York, NY (US)

(73) Assignee: MEMORIAL SLOAN-KETTERING CANCER CENTER, New York, NY (US)

(21) Appl. No.: 17/193,367

(22) Filed: Mar. 5, 2021

Related U.S. Application Data
(63) Continuation of application No. PCT/US2019/049771, filed on Sep. 5, 2019.
(60) Provisional application No. 62/884,736, filed on Aug. 9, 2019.
(60) Provisional application No. 62/819,141, filed on Mar. 15, 2019.
(60) Provisional application No. 62/727,463, filed on Sep. 5, 2018.

(51) Int. Cl. G01N 33/50; C07K 16/30; C07K 14/705; C07K 14/725

(52) U.S. Cl. CPC G01N 33/505; C07K 16/30; G01N 2800/7004; C07K 14/7051; C07K 14/70521

(57) ABSTRACT

The present disclosure relates to methods, kits and systems for assessing cytotoxicity of immunoresponsive cells using a pleural effusion. The present disclosure also relates to methods, kits and systems for assessing effect of an immunotherapeutic agent on cytotoxicity of immunoresponsive cells using a pleural effusion.

### CROSS-REFERENCED TO RELATED APPLICATIONS

This application is Continuation of International Patent Application No. PCT/US2019/049771, filed Sep. 5, 2019, which claims priority to U.S. Provisional Application No. 62/727,463 filed Sep. 5, 2018, U.S. Provisional Application No. 62/819,141 filed Mar. 15, 2019, and U.S. Provisional Application No. 62/884,736 filed Aug. 9, 2019, the contents of each of which are incorporated by reference in their entireties, and to each of which priority is claimed.

### GRANT INFORMATION

This invention was made with government support under Grant Nos. CA236615-01, CA235667-01A1, CA217169-01-A1, and CA213139 awarded by National Cancer Institute of National Institutes of Health, and Grant Nos.: CA180889, CA170630, LC160212, and BC132124 awarded by United States Department of Defense. The government has certain rights in the invention.

### SEQUENCE LISTING

The specification further incorporates by reference the Sequence Listing submitted herewith via EFS on Mar. 5, 2021. Pursuant to 37 C.F.R. § 1.52(e)(5), the Sequence Listing text file, identified as 072734_1225_SL.txt, is 9,370 bytes and was created on Mar. 3, 2021. The Sequence Listing electronically filed herewith, does not extend beyond the scope of the specification and thus does not contain new matter.

### 1. INTRODUCTION

The present disclosure relates to methods, kits and systems for assessing cytotoxicity of immunoresponsive cells using a pleural effusion. The present disclosure also relates to methods, kits and systems for assessing effects of an immunotherapeutic agent on cytotoxicity of immunoresponsive cells using a pleural effusion.

### 2. BACKGROUND

Discovery of new drug therapies usually starts with in vitro model systems of cells and tissues to identify the etiology/pathogenesis of disease states, and novel therapeutic strategies that might interfere with the pathologic processes demonstrated in the cells or tissues. Potential therapeutic agents are further tested in vivo in more complex systems including animal models, e.g., mice, dogs, and monkeys. The closer an animal model resembles the pathophysiology of a human disease, the more predictive is the model on the human response to the tested therapeutic agents. However, for certain therapeutics, no good animal models are available to predict the in vivo effects of a therapeutics inhuman patients. In particular, for immunotherapeutic agents for treating solid tumors, tumor microenvironment significantly impacts the activities of these agents in human patients. However, it is difficult to recreate the tumor microenvironment of humans in animal models.

Thus, there remains a need for methods of evaluating immunotherapeutic agents preclinically under conditions that mimic tumor microenvironment in humans.

### 3. SUMMARY OF THE INVENTION

The present disclosure relates to methods, kits and systems for assessing cytotoxicity of immunoresponsive cells using a pleural effusion (e.g., a pleural effusion collected from a subject). The present disclosure also relates to methods, kits and systems for assessing effect of an immunotherapeutic agent on cytotoxicity of immunoresponsive cells using a pleural effusion (e.g., a pleural effusion collected from a subject).

The present disclosure provides methods for assessing cytotoxicity of an immunoresponsive cell. In certain embodiments, the method comprises:
(a) culturing an immunoresponsive cell in a pleural effusion;
(b) contacting the immunoresponsive cell with a target cell; and
(c) measuring a status of the target cell contacted with the immunoresponsive cell,
wherein the status of the target cell indicates the cytotoxicity of the immunoresponsive cell.

In certain embodiments, (b) comprises culturing the target cell and immunoresponsive cell in the pleural effusion.

Furthermore, the present disclosure provides methods for assessing the effect of an immunotherapeutic agent on cytotoxicity of an immunoresponsive cell. In certain embodiments, the method comprises:
(a) culturing an immunoresponsive cell in a first pleural effusion;
(b) contacting the immunoresponsive cell with a target cell;
(c) measuring a status of the target cell contacted with the immunoresponsive cell, wherein the status indicates the cytotoxicity of the immunoresponsive cell;
(d) culturing the immunoresponsive cell in a second pleural effusion;
(e) contacting an immunotherapeutic agent with the immunoresponsive cell;
(f) contacting the immunoresponsive cell with the target cell;
(g) measuring the status of the target cell contacted with the immunoresponsive cell; and
(h) comparing the status measured in (g) with the status measured in (c), wherein a change between the status measured in (g) and the status measured in (c) indicates that the immunotherapeutic agent has an effect on cytotoxicity of the immunoresponsive cell.

In certain embodiments, (b) comprises culturing the target cell and immunoresponsive cell in the first pleural effusion. In certain embodiments, (f) comprises culturing the target cell and immunoresponsive cell in the second pleural effusion.

In certain embodiments, (e) comprises culturing the immunotherapeutic agent and the immunoresponsive cell in the second pleural effusion.

Furthermore, the present disclosure provides kits and systems for assessing cytotoxicity of an immunoresponsive cell. In certain embodiments, the kit or system comprises a pleural effusion, an immunoresponsive cell, and a target cell.

In certain embodiments, the kit or system further comprises instructions for assessing the cytotoxicity of the immunoresponsive cell. In certain embodiments, the instructions comprise:
(a) culturing the immunoresponsive cell in a pleural effusion;
(b) contacting the immunoresponsive cell with the target cell;
(c) measuring a status of the target cell contacted with the immunoresponsive cell,
wherein the status of the target cell indicates the cytotoxicity of the immunoresponsive cell.

The present disclosure also provides kits and systems for assessing the effect of an immunotherapeutic agent on cytotoxicity of an immunoresponsive cell. In certain embodiments, the kit or system comprises: a pleural effusion, an immunoresponsive cell, and a target cell.

In certain embodiments, the kit or system further comprises instructions for assessing the effect of the immunotherapeutic agent on the cytotoxicity of the immunoresponsive cell. In certain embodiments, the instructions comprise:
(a) culturing the immunoresponsive cell in a first pleural effusion;
(b) contacting the immunoresponsive cell with the target cell;
(c) measuring a status of the target cell contacted with the immunoresponsive cell, wherein the status indicates the cytotoxicity of the immunoresponsive cell;
(d) culturing the immunoresponsive cell in a second pleural effusion;
(e) contacting an immunotherapeutic agent with the immunoresponsive cell;
(f) contacting the immunoresponsive cell with the cell comprising the tumor antigen or pathogen antigen;
(g) measuring the status of the target cell contacted with the immunoresponsive cell; and
(h) comparing the status measured in (g) with the status measured in (c), wherein a change between the status measured in (g) and the status measured in (c) indicates that the immunotherapeutic agent has an effect on cytotoxicity of the immunoresponsive cell.

In certain various embodiments, the target cell comprises a tumor antigen or a pathogen antigen.

In certain various embodiments, the status of the target cell is selected from the group consisting of cell death, cell proliferation, cell apoptosis, cell necrosis, cell autophagy, cell lysis, cell growth arrest, cell antigen expression suppression, cell chemokine receptor expression, cell chemokine secretion, cell receptor (e.g., PD-1, PD-2) expression, cell ligand (e.g., PD-L1, PD-L2) expression, and combinations thereof. In certain various embodiments, the status of the target cell is measured by a Cr⁵¹ release assay, a bioluminescence assay, a flow cytometry assay, an impedance assay, an apoptosis assay, an assay measuring chemokine secretion, an assay measuring cell ligand expression, an assay measuring cell receptor expression, or a combination of the foregoing. In certain various embodiments, the status of the target cells is measured by an impedance assay.

In certain various embodiments, the pleural effusion is obtained from a subject. In certain various embodiments, the pleural effusion is obtained from two or more subjects. In certain various embodiments, the subject suffers from cancer. In certain various embodiments, the pleural effusion is obtained from a subject who previously received an anti-cancer agent. In certain embodiments, the anti-cancer agent is selected from the group consisting of an immune checkpoint inhibitor, cytokines, oncolytic virus, T cells, dendritic cells, bispecific antibodies, BiTEs, immunotoxins, and combinations thereof. In certain various embodiments, the anti-cancer agent comprises an immune checkpoint inhibitor. In certain various embodiments, the pleural effusion is substantially free of immune cells. In certain embodiments, the pleural effusion comprises immune cells. In certain various embodiments, the immune cells are selected from the group consisting of T cells, B cells, Nature Killer (NK) cells, neutrophils, macrophages, dendritic cells, and combinations thereof. In certain various embodiments, the T cells are selected from a group consisting of cytotoxic T lymphocytes (CTLs), regulatory T cells (Tregs), and Natural Killer T (NKT) cells, and combinations thereof. In certain various embodiments, the pleural effusion has an immunosuppressive effect.

In certain various embodiments, the method comprises culturing the immunoresponsive cells in the pleural effusion for at least about 30 minutes before its initial contact with the target cell. In certain various embodiments, the method comprises culturing the immunoresponsive cells in the pleural effusion for up to about 72 hours before its initial contact with the target cell. In certain various embodiments, the method comprises culturing the immunoresponsive cells in the pleural effusion for about 24 hours before its initial contact with the target cell.

In certain various embodiments, the method comprises measuring the status of the target cell at least about 1 hour from the initial contact of the immunoresponsive cell with the target cell. In certain various embodiments, the method comprises measuring the status of the target cell no later than about 72 hours from the initial contact of the immunoresponsive cell with the target cell. In certain various embodiments, the method comprises measuring the status of the target cell about 18 hours from the initial contact of the immunoresponsive cell with the target cell.

In certain embodiments, the immunoresponsive cell comprises a receptor that binds to an antigen. In certain embodiments, the receptor is a T-cell receptor (TCR) or a chimeric antigen receptor (CAR). In certain embodiments, the CAR comprises an extracellular antigen-binding domain, a transmembrane domain, and an intracellular signaling domain. In certain embodiments, the intracellular signaling domain further comprises a co-stimulatory signaling domain. In certain embodiments, the extracellular antigen-binding domain specifically binds to mesothelin. In certain embodiments, the extracellular antigen-binding domain comprises a V_{H} CDR1 comprising amino acids having the sequence set forth in SEQ ID NO: 1, a V_{H} CDR2 comprising the amino acid sequence set forth in SEQ ID NO: 2, a V_{H} CDR3 comprising the amino acid sequence set forth in SEQ ID NO: 3, a V_{L} CDR1 comprising the amino acid sequence set forth in SEQ ID NO: 4, a V_{L} CDR2 comprising the amino acid sequence set forth in SEQ ID NO: 5, and a V_{L} CDR3 comprising the amino acid sequence set forth in SEQ ID NO: 6. In certain embodiments, the transmembrane domain comprises a CD28 polypeptide. In certain embodiments, the CD28 polypeptide comprises or has the amino acid sequence set forth in SEQ ID NO: 16. In certain embodiments, the intracellular signaling domain comprises a CD3ζ polypeptide. In certain embodiments, the CD3ζ polypeptide comprises or has amino acids 52 to 164 of SEQ ID NO: 12. In certain embodiments, the co-stimulatory signaling region comprises a CD28 polypeptide. In certain embodiments, the CD28 polypeptide comprises or has the amino acid sequence set forth in SEQ ID NO: 14.

In certain embodiments, the antigen to which the receptor binds is a tumor antigen or a pathogen antigen. In certain embodiments, the antigen to which the receptor binds is a tumor antigen. In certain embodiments, the tumor antigen is mesothelin. In certain embodiments, the immunoresponsive cell can be selected from the group consisting of T cells, Natural Killer (NK) cells, human embryonic stem cells, and pluripotent stem cells from which lymphoid cells may be differentiated. In certain embodiments, the immunoresponsive cell is a T cell. In certain embodiments, the T cell is selected from the group consisting of cytotoxic T lymphocytes (CTLs), regulatory T cells (Tregs), and Natural Killer T (NKT) cells.

### 4. BRIEF DESCRIPTION OF THE DRAWINGS

FIG. 1 depicts the available assays for translational research and that the methods and systems disclosed herein, e.g., ex-vivo plural effusion culture system (ePECS) can be used to replace the available assays for translational research, and to fulfill unmet need for non-existing human model system to investigate immunotherapeutic agents.

FIG. 2 shows the cell components of the malignant plural effusion (MPE) obtained from patients.

FIG. 3 shows that the cells contained in the MPE, e.g., tumor cells, and a full complement of immune cells including T cells, Tregs, B cells, NK cells, neutrophils, macrophages, and dendritic cells, and cytokines.

FIG. 4 shows the optimal seeding concentration of tumor cells in the impedance assay for measuring T-cell cytotoxicity. The optimal seeding concentration of tumor cells for the assay was between 10,000-20,000 cells/well.

FIG. 5 shows that T cells had a concentration-dependent but minimal effect on impedance. Impact of T cells at low E:T ratios on cell index was negligible.

FIG. 6 shows that CAR T-cell cytotoxicity demonstrated a concentration-dependent drop in cell index.

FIG. 7 shows that the impedance assay was comparable to (equally effective as) Chromium (Cr) release cytotoxicity T lymphocyte (CTL) assay.

FIG. 8 shows cell-free pleural effusions influenced CAR T-cell efficacy. CAR T cells obtained from different donors were influenced differently by pleural effusions from different patients.

FIG. 9 shows differential cytotoxicity of CAR T cells cultured in a cell-free pleural effusion from different patients.

FIG. 10 shows the impact of pleural effusion cells on cell index. MPE cells were shown to have a concentration-dependent impact on cell index.

FIG. 11 shows the impact of pleural effusion cells on tumor growth. MPE cells were shown to inhibit tumor growth in a concentration-dependent manner.

FIG. 12 shows that functional exhaustion relating to cytotoxicity was observed following repeat stimulations of CAR T-cells.

FIG. 13 shows the results of antigen stress test for CAR T-cells, which measured CAR T-cell cytotoxicity following exposure to repeat antigen stimulation.

FIG. 14 shows the results of antigen stress test for CAR T-cells.

FIGS. 15A and 15B show the influence of TGF-β on tumor growth in cell line 1 (FIG. 15A) and cell line 2 (FIG. 15B).

FIGS. 16A and 16B show that recombinant TGF-β inhibited CAR T-cell efficacy, measured by cytolysis (FIG. 16A) and cell index (FIG. 16B).

FIG. 17 shows TGF-β inhibition depended on activation status of CAR T cells, and depended on the prior antigen stimulation of CART cells.

FIG. 18 shows cytotoxicity kinetics of CAR T-cells in the presence of TGF-β, and the rescue of CAR T-cell cytotoxicity in the presence of anti-TGFβ antibody.

FIGS. 19A-19B show that CAR T-cell 1 (FIG. 19A) and CAR T-cell 2 (FIG. 19B) were equally inhibited by TGF-β.

FIG. 20 shows the impact of pleural effusion cells and cell-free effusion on target and CAR T cells.

FIG. 21 shows the impact of pleural effusion cells and cell-free effusion on target and CAR T cells.

FIG. 22 shows that the composite of MPE, e.g., it is composed of immune and tumor cell-derived soluble factors.

FIG. 23 shows an exemplary scheme of the methods and systems for evaluating the activity of antigen-receptors in accordance with certain embodiments of the present disclosure.

FIG. 24 shows gene expression of CAR T cells varied upon exposure to MPE.

FIG. 25 shows MPE induced downregulation of effector gene expression.

FIG. 26 shows that CAR T-cell cytotoxicity was associated with soluble factors present in MPE.

FIG. 27 shows the specifics of soluble PD-1 and PD-L1 used.

FIG. 28 shows the timeline of experiment.

FIG. 29 shows transduction of M28z and M28z-PD1DNR in T-cell day 9 (day of eCTL; cells for flow incubated separately at 2×10⁵/200 ul in 96W plate 3 days before).

FIG. 30 shows MSLN expression of A549GM (non-small cell lung cancer cells) and MGM (mesothelioma cells).

FIG. 31 shows cell counts after 3 day incubation with soluble factors (no antigen) (before plating in eCTL).

FIG. 32 shows that at E:T ratio 3:1, M28z killed faster than M28z-PD1DNR (no soluble factors added).

FIG. 33 shows that sPD-1 and sPD-L1 did not affect M28z and M28z-PD1DNR cytotoxicity: A549GM.

FIG. 34 shows that sPD-1 and sPD-L1 did not affect M28z and M28z-PD1DNR cytotoxicity: MGM.

FIGS. 35A and 35B each show that M28z and M28z-PD1DNR were about equally inhibited by TGFβ1: A549GM FIG. 36 shows that TGFβ1 inhibition in UT T cells.

FIG. 37 shows that TGFβ1 inhibition normalized to cell index of tumor cells and UT as mock control.

FIG. 38 shows the results of combining TGFβ with sPD-1 and sPD-L1: A549GM.

FIG. 39 shows the results of combining TGFβ with sPD-1 and sPD-L1: MGM.

FIG. 40 shows the comparison M28z vs M28z-PD1DNR towards PD1 and TGFβ1 (A549GM).

FIG. 41 shows that PD-L1-Fc was biologically active but only at 1000 fold higher concentration than used in eCTL.

FIG. 42 shows that cell-free MPE as an immunosuppressive system to study CAR T-cell efficacy.

FIG. 43 shows treatment conditions.

FIG. 44 shows flow cytometry A549GM analysis.

FIG. 45 shows flow cytometry T-cell analysis. Columns represent antigen activation status of CAR T cells, whether TGFβ antibody was added or not, percentages of total T cells with CAR expression, and percentages of CD4 and CD8 CAR T cells with CAR expression.

FIG. 46 shows M28z in RPMI+10% FCS with recombinant TGFβ1 (no IL-2).

FIG. 47 shows M28z in RPMI+10% FCS with recombinant TGFβ1 (no IL-2).

FIG. 48 shows that addition of IL-2 improved cytotoxicity of M28z, with no TGFβ1 addition.

FIG. 49 shows the results in A549GM supernatant.

FIG. 50 shows summary A549GM supernatant.

FIG. 51 shows results of cell-free MPE11.

FIG. 52 shows summary cell-free MPE11.

FIG. 53 shows MPE 41 tumor panel gating strategy.

FIG. 54 shows MPE 41 results with FMO.

FIG. 55 shows MSLN/GFP expression of A549GM and MGM before plating.

FIG. 56 shows M28z transduction efficacy. Donor: ZTBD1; frozen T cells, day 8; last IL-2: after thawing until exp; sup from May 5, 2017, undiluted.

FIG. 57 shows impact of PE41 on A549GM and T-cell mediated killing.

FIG. 58 shows impact of PE41 on MGM and T-cell mediated killing.

FIG. 59 shows comparison CAR T cell efficacy in presence of PE41 cells (normalized).

FIGS. 60A and 60B show the experimental plan, including timeline (FIG. 60A) and groups (FIG. 60B).

FIG. 61 shows plate layout.

FIG. 62 shows ZEN microscope brightfield and GFP merged images over time.

FIG. 63 shows ZEN microscope brightfield and GFP merged images over time, in cells incubated in MPE 51.

FIG. 64 shows ZEN microscope brightfield and GFP merged images over time, in cells incubated in MPE 55.

FIG. 65 shows ZEN microscope brightfield and GFP merged images over time, in cells incubated in MPE 81.

FIG. 66 shows representative flow cytometry gating strategy.

FIG. 67 shows representative flow cytometry gating strategy.

FIG. 68 shows flow cytometry analysis of cell populations over time.

FIG. 69 shows flow cytometry analysis of cell populations over time.

FIG. 70 shows flow cytometry analysis of cell populations over time.

FIG. 71 shows assessing MGM tumor cell population through luciferin activity assay.

FIGS. 72A and 72B show the experimental plan, including timeline (FIG. 72A) and groups (FIG. 72B).

FIG. 73 shows plate layout.

FIG. 74 shows ZEN microscope brightfield and GFP merged images over time.

FIG. 75 shows representative flow cytometry gating strategy (immune cells).

FIG. 76 shows representative flow cytometry gating strategy (MGM cells).

FIG. 77 shows flow cytometry analysis of cell populations over time.

FIG. 78 shows flow cytometry analysis of cell populations over time.

FIG. 79 shows flow cytometry analysis of cell populations over time.

FIG. 80 shows assessing MGM tumor cell population through GFP and luciferin activity assay.

FIGS. 81A and 81B show the experimental plan, including timeline (FIG. 81A) and groups (FIG. 81B).

FIG. 82 shows plate layout.

FIG. 83 shows ZEN microscope brightfield and GFP merged images over time. Cells were incubated with or without different doses of radiation or with or without addition of CSF1 factor at different concentrations.

FIG. 84 shows representative flow cytometry analysis (D2), following addition of different doses of CSF1.

FIG. 85 shows representative flow cytometry analysis (D3), following addition of different doses of CSF1.

FIG. 86 shows flow cytometry analysis of cell populations, following different doses of radiation.

FIG. 87 shows flow cytometry analysis of cell populations, following different doses of radiation.

FIG. 88 shows assessing MGM tumor cell population through luciferin activity assay.

### 5. DETAILED DESCRIPTION

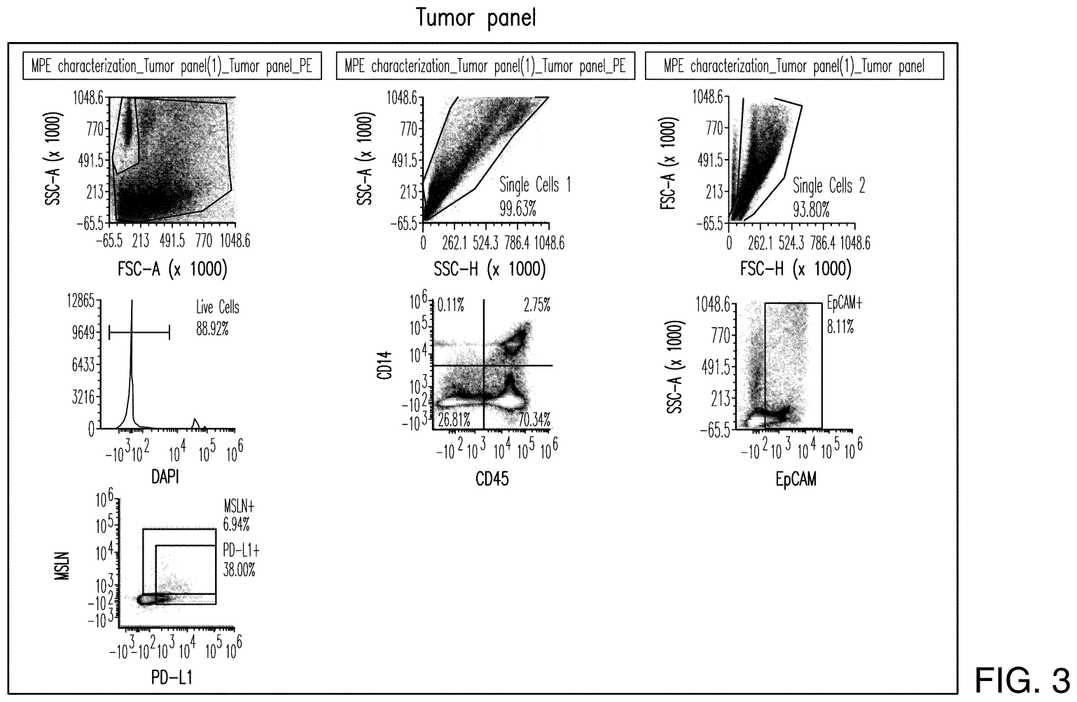

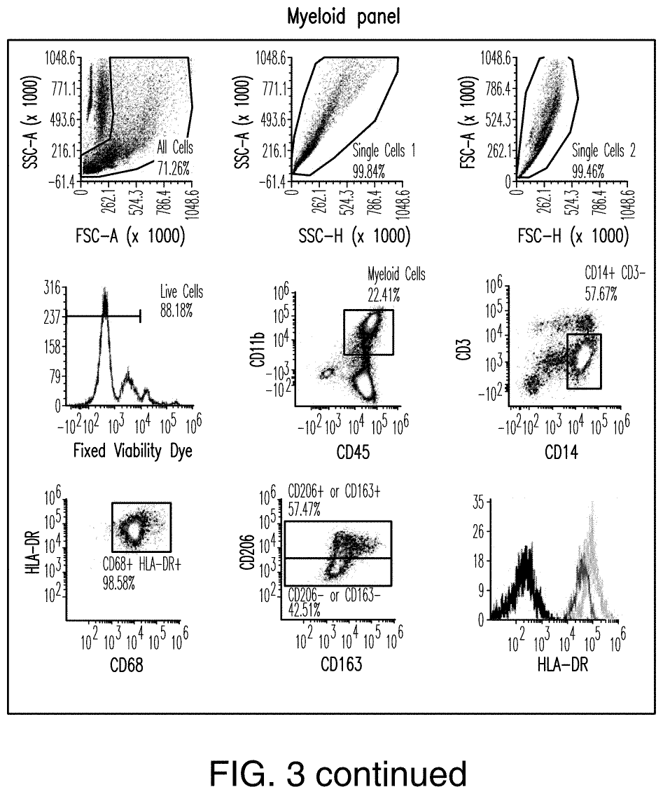

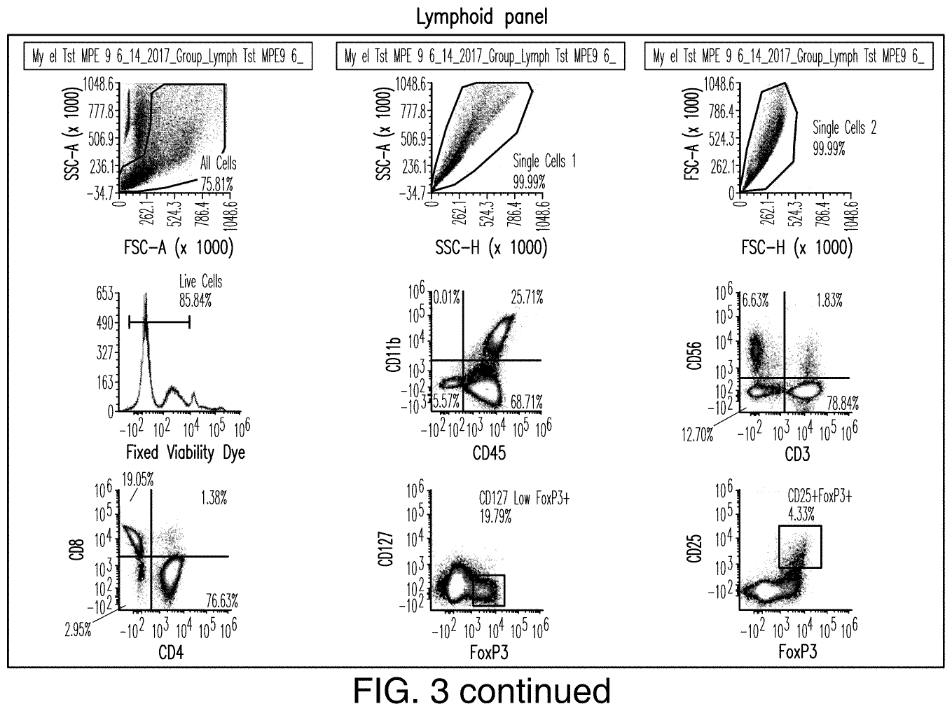

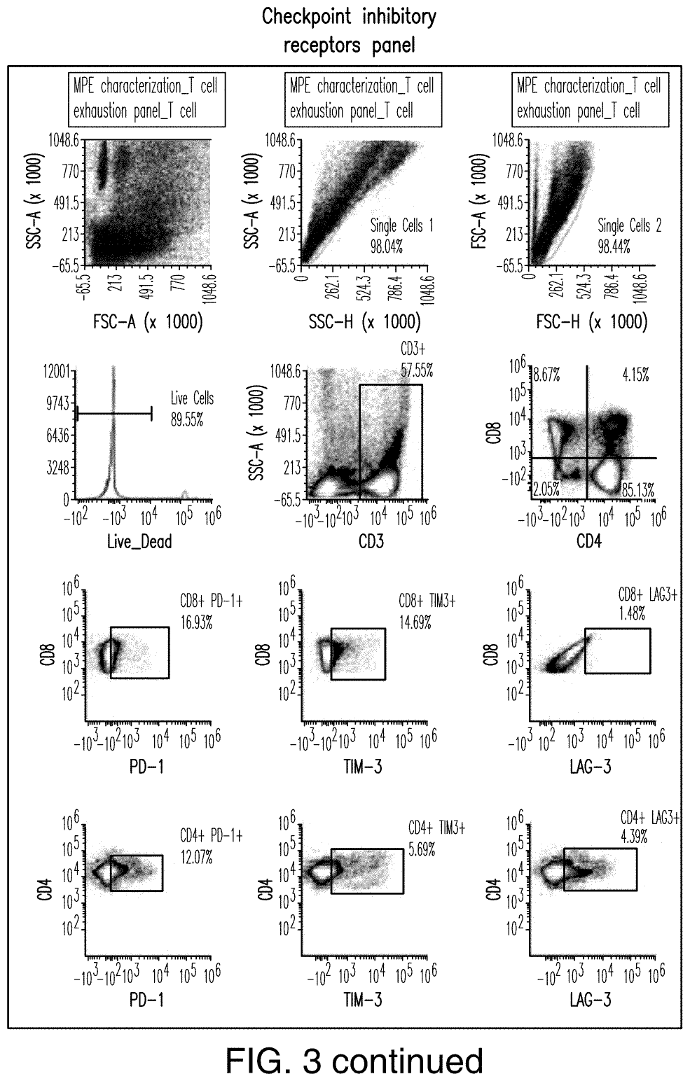

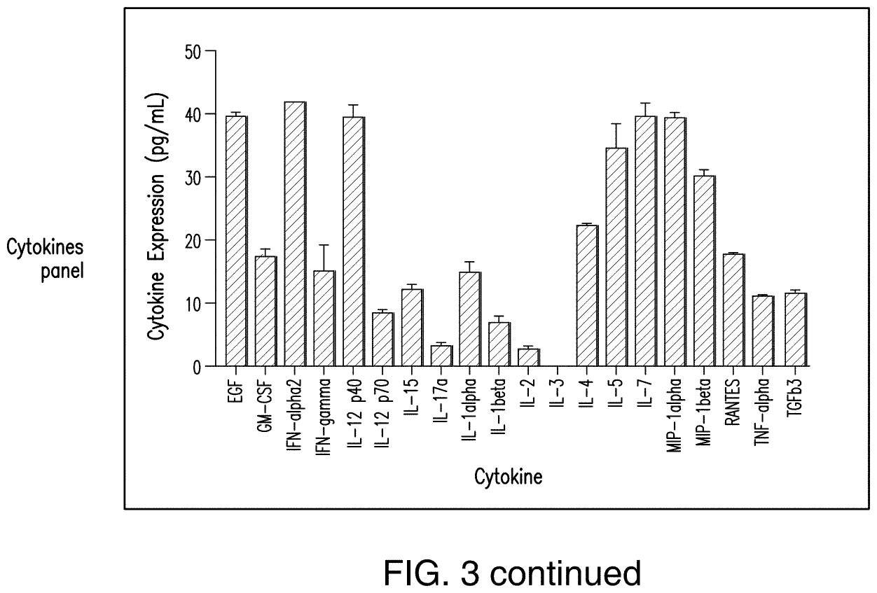

The present disclosure relates to methods, kits and systems for assessing cytotoxicity of immunoresponsive cells using a pleural effusion (e.g., a pleural effusion collected from a subject). The present disclosure also relates to methods, kits and systems for assessing effect of an immunotherapeutic agent on cytotoxicity of immunoresponsive cells using a pleural effusion (e.g., a pleural effusion collected from a subject). The present disclosure is partly based on the discovery that a pleural effusion obtained from a subject (e.g., a subject suffering from cancer) includes components of tumor microenvironment, such as tumor cells, a full complement of immune cells (e.g., T-cells, Tregs, B-cells, NK cells, neophiles, macrophages, and/or dendritic cells), and cytokines (see FIG. 3). Thus, a pleural effusion can be used in an in vitro system or kit to predict the in vivo performance of immunoresponsive cells (e.g., cytotoxicity toward target cells, e.g., cells comprising a tumor antigen or pathogen antigen) and immunotherapeutic agents (e.g., impact of immunotherapeutic agents on the cytotoxicity of immunoresponsive cells). Additionally, such in vitro system or kit provide advantages over animal testing, such as ethical issues, time-consuming protocols, and high cost.

Non-limiting embodiments of the present disclosure are described by the present specification and Examples.

For purposes of clarity of disclosure and not by way of limitation, the detailed description is divided into the following subsections:
5.1 Definitions;
5.2 Pleural effusions;
5.3. Uses of Pleural Effusions;
5.4. Immunoresponsive cells; and
5.5 Kits and Systems.

### 5.1. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “pleural effusion” refers to the excess fluid building up between the layers of the pleura outside the lungs. The causes of a pleural effusion include, but are not limited to, heart failure, pulmonary embolism, cirrhosis, post open heart surgery, pneumonia, cancer, pulmonary embolism, kidney disease, inflammatory disease, tuberculosis, autoimmune disease, bleeding (due to chest trauma), chylothorax (due to trauma), rare chest and abdominal infections, asbestos pleural effusion (due to exposure to asbestos), Meigs syndrome (due to a benign ovarian tumor), and ovarian hyperstimulation syndrome. Pleural effusion may also be caused by certain medications, abdominal surgery and radiation therapy. Pleural effusion may occur in several types of cancer including lung cancer, breast cancer and lymphoma. In certain embodiments, the pleural effusion may be malignant (cancerous), or may be a direct result of chemotherapy or immunotherapy.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes.

As used herein, the term “ligand” refers to a molecule that binds to a receptor. In particular, the ligand binds a receptor on another cell, allowing for cell-to-cell recognition and/or interaction.

As used herein, the term “immunoresponsive cell” refers to a cell that functions in an immune response or a progenitor, or progeny thereof.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “isolated,” “purified,” or “biologically pure” refers to material that is free to varying degrees from components which normally accompany it as found in its native state. “Isolate” denotes a degree of separation from original source or surroundings. As used herein, the term “isolated cell” refers to a cell that is separated from the molecular and/or cellular components that naturally accompany the cell. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “receptor” refers to mean a polypeptide, or portion thereof, present on a cell membrane that selectively binds one or more ligand.

As used herein, the term “recognize” is meant selectively binds to a target. A T cell that recognizes a virus or tumor typically expresses a receptor that binds an antigen expressed by the virus or tumor.

As used herein, the term “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

As used herein, the term “specifically binds” or “specifically binds to” or “specifically target” is meant a polypeptide or fragment thereof that recognizes and binds a biological molecule of interest (e.g., a polypeptide), but which does not substantially recognize and bind other molecules in a sample, for example, a biological sample, which naturally includes a polypeptide of the invention.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

An “effective amount” of a substance as that term is used herein is that amount sufficient to effect beneficial or desired results, including clinical results, and, as such, an “effective amount” depends upon the context in which it is being applied. An effective amount can be administered in one or more administrations.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more sign or symptoms, diminishment of extent of disease, stabilized (i.e., not worsening) state of disease, prevention of disease, delay or slowing of disease progression, and/or amelioration or palliation of the disease state. The decrease can be a 10%, 20%, 30%, 40%, 50%, 60%, 70%, 80%, 90%, 95%, 98% or 99% decrease in severity of complications or symptoms. “Treatment” can also mean prolonging survival as compared to expected survival if not receiving treatment.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “ex vivo” refers to procedures done with tissues taken from an organism in an external environment.

### 5.2. Pleural Effusions

In certain embodiments, the pleural effusion is obtained from a subject. The pleural effusion can be obtained from one subject or two or more subjects.

In certain embodiments, the pleural effusion is obtained from a subject having a disease or disorder. Non-limiting examples of diseases or disorders include heart failure, pulmonary embolism, cirrhosis, post open heart surgery, pneumonia, cancers, pulmonary embolism, kidney diseases, inflammatory diseases, tuberculosis, autoimmune diseases, bleeding (due to chest trauma), chylothorax (due to trauma), rare chest and abdominal infections, asbestos pleural effusion (due to exposure to asbestos), Meigs syndrome (due to a benign ovarian tumor), ovarian hyperstimulation syndrome, and combinations thereof. In certain embodiments, the pleural effusion is obtained from a subject who received an abdominal surgery, radiation therapy, chemotherapy, or a combination thereof.

In certain embodiments, the pleural effusion is obtained from a subject suffering from cancer. Non-limiting examples of cancers include lung cancer, breast cancer, ovarian cancer, leukemias, lymphomas, colorectal cancer, prostate cancer, sarcoma, mesothelioma, and combinations thereof.

In certain embodiments, the pleural effusion is obtained from a subject who receives or previously received an anti-cancer agent. Non-limiting examples of anti-cancer agents include chemotherapeutic agents, radiotherapeutic agents, cytokines, oncolytic virus, T cells, dendritic cells, bispecific antibodies, BiTEs, immunotoxins, anti-angiogenic agents, apoptosis-inducing agents, anti-cancer antibodies, targeted drugs, checkpoint inhibitors, agents that are capable of promoting the activity of an immune system, and combinations thereof. In certain embodiments, the anti-cancer agent is a radiotherapeutic agent. In certain embodiments, the anti-cancer agent is an agent that is capable of promoting the activity of the immune system, including but not limited to interleukins (ILs, e.g., interleukin 2), interferon, anti-CTLA4 antibodies, anti-PD-1 antibodies, oncolytic virus, T cells, dendritic cells, bispecific antibodies, BiTEs, immunotoxins, and anti-PD-L1 antibodies. In certain embodiments, the anti-cancer agent is an immune checkpoint inhibitor. Any suitable immune checkpoint inhibitors known in the art can be used with the present disclosure. In certain embodiments, the immune checkpoint inhibitor is an anti-CTLA4 antibody, an anti-PD-1 antibody, an anti-PD-L1 antibody, an anti-BTLA antibody, an anti-TIM3 antibody, or an anti-LAG-3 antibody.

In certain embodiments, the pleural effusion comprises cells. In certain embodiments, the pleural effusion comprises tumor cells (e.g., cancer cells). In certain embodiment, the pleural effusion comprises immune cells. In certain embodiments, the immune cells are selected from the group consisting of T cells (e.g., T-regs), B-cells, NK-cells, neutrophils, macrophages, dendritic cells, and combinations thereof. In certain embodiments, the cells are from the subject from whom the pleural effusion is obtained.

In certain embodiments, the pleural effusion comprises proteins. In certain embodiments, the proteins comprise cytokines. In certain embodiments, the cytokines are selected from the group consisting of EGF, GM-CSF, IFN-alpha2, IFN-gamma, IL-12 p40, IL-12 p70, IL-15, IL-17a, IL-1alpha, IL-1beta, IL-2, IL-3, IL-4, IL-5, IL-7, MIP-1alpha, MIP-1beta, RANTES, TNF-alpha, TGFβ3, and combinations thereof. In certain embodiments, the proteins (e.g., cytokines) are from the subject from whom the pleural effusion is obtained.

In certain embodiments, the pleural effusion is substantially free or free of cells. For example, the pleural effusion obtained from the subject is further processes to remove cells.

In certain embodiments, the pleural effusion does not have an immunosuppressive effect. In certain embodiments, the pleural effusion has an immunosuppressive effect. In certain embodiments, the immunosuppressive effect of the pleural effusion corresponds to the immunosuppressive microenvironment in which the tumor cells are located in vivo in the subject. In many cases, a major hurdle for cancer immunotherapy is that the tumor cells are located in an immunosuppressive microenvironment. Under the immunosuppressive environment, immunoresponsive cells (e.g., T-cells) cannot fully achieve their tumoricidal potential in vivo. Thus, the number of tumor antigen-specific T cells present in the periphery does not readily translate to tumor cell killing. In certain embodiments, the presently disclosed pleural effusions carry factors that contribute to the in vivo immunosuppressive microenvironment, and thus, can be used ex vivo and in vitro for predicting the cytotoxicity (e.g., tumor cell killing ability) of immunoresponsive cells in vivo.

### 5.3. Uses of Pleural Effusions

As disclosed in Section 5.2, a pleural effusion (e.g., one obtained from a subject) can be used to predict the cytotoxicity of immunoresponsive cells in vivo. The present disclosure provides methods for assessing cytotoxicity of immunoresponsive cells (e.g., cytotoxicity of immunoresponsive cells in vivo). In certain embodiments, the method comprises: (a) culturing an immunoresponsive cell in a pleural effusion; (b) contacting the immunoresponsive cell with a target cell; (c) measuring a status of the target cell, wherein the status of target cell indicates the cytotoxicity of the immunoresponsive cell. In certain embodiments, the target cell comprises a tumor antigen or a pathogen antigen.

Furthermore, the pleural effusion disclosed herein can also be used to assess the effect of an immunotherapeutic agent on cytotoxicity of immunoresponsive cells. In certain embodiments, the method comprises:
(a) culturing an immunoresponsive cell in a first pleural effusion;
(b) contacting the immunoresponsive cell with a target cell;
(c) measuring a status of the target cell contacted with the immunoresponsive cell, wherein the status indicates the cytotoxicity of the immunoresponsive cell;
(d) culturing the immunoresponsive cell in a second pleural effusion;
(e) contacting an immunotherapeutic agent with the immunoresponsive cell;
(f) contacting the immunoresponsive cell with the target cell;
(g) measuring the status of the target cell contacted with the immunoresponsive cell; and
(h) comparing the status measured in (g) with the status measured in (c), wherein a change between the status measured in (g) and the status measured in (c) indicates that the immunotherapeutic agent has an effect on cytotoxicity of the immunoresponsive cell.

In certain embodiments, contacting the immunoresponsive cell with the target cell comprises culturing the immunoresponsive cell and the target cell in the pleural effusion (e.g., in the first pleural effusion and/or the second pleural effusion).

In certain embodiments, contacting the immunoresponsive cell with the immunotherapeutic agent comprises culturing the immunoresponsive cell and the immunotherapeutic agent in the second pleural effusion.

In certain embodiments, the immunoresponsive cell is cultured in the pleural effusion (e.g., the first pleural effusion, and/or the second pleural effusion (optionally together with the immunotherapeutic agent)) for a period of time before its initial contact with the target cell. In certain embodiments, the immunoresponsive cell is cultured in the pleural effusion (e.g., the first and/or second pleural effusion)) for at least about 30 minutes, at least about 1 hour, at least about 5 hours, at least about 10 hours, at least about 15 hours, at least about 20 hours, at least about 24 hours, or at least about 2 days, before its initial contact with the target cell. In certain embodiments, the immunoresponsive cell is cultured in the pleural effusion (e.g., the first pleural effusion, and/or the second pleural effusion (optionally together with the immunotherapeutic agent)) for up to about 20 hours, up to about 24 hours, up to about 2 days, or up to about 3 days before its initial contact with the target cell.

In certain embodiments, the immunoresponsive cell is cultured in the pleural effusion (e.g., the first pleural effusion, and/or the second pleural effusion (optionally together with the immunotherapeutic agent)) for between about 2 hours and about 5 days, between about 2 hours and about 2 days, between about 1 day and about 2 days, between about 10 hours and about 36 hours, between about 10 hours and about 30 hours, between about 15 hours and about 30 hours, between about 15 hours and about 24 hours, or between about 24 hours and about 30 hours before its initial contact with the target cell.

In certain embodiments, the immunoresponsive cell is cultured in the pleural effusion (e.g., the first pleural effusion, and/or the second pleural effusion (optionally together with the immunotherapeutic agent)) for about 4 hours, about 6 hours, about 8 hours, about 10 hours, about 12 hours, about 14 hours, about 16 hours, about 18 hours, about 20 hours, about 22 hours, about 24 hours, about 2 days, about 3 days, about 4 days, or about 5 days before its initial contact with the target cell. In certain embodiments, the immunoresponsive cell is cultured in the pleural effusion (e.g., the first pleural effusion, and/or the second pleural effusion (optionally together with the immunotherapeutic agent)) for about 24 hours before its initial contact with the target cell.

In certain embodiments, the status of the target cell is measured at least about 1 hour, at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, at least about 22 hours, at least about 24 hours, at least about 26 hours, at least about 28 hours, at least about 30 hours, at least about 32 hours, at least about 36 hours, at least about 38 hours, at least about 40 hours, at least about 42 hours, at least about 44 hours, at least about 46 hours, or at least about 48 hours from the initial contact of the target cell with the immunoresponsive cell. In certain embodiments, the status of the target cell is measured no later than about 10 hours, no later than about 15 hours, no later than about 20 hours, no later than about 24 hours, no later than about 2 days, or no later than about 3 days from the initial contact of the target cell with the immunoresponsive cell.

In certain embodiments, the status of the target cell is measured between about 2 hours and about 5 days, between about 2 hours and about 2 days, between about 2 hours and about 24 hours, between about 1 day and about 2 days, between about 10 hours and about 20 hours, between about 15 hours and about 20 hours, between about 10 hours and about 24 hours, or between about 15 hours and about 24 hours from the initial contact of the target cell with the immunoresponsive cell. In certain embodiments, the status of the target cell is measured between about 10 hours and about 20 hours from the initial contact of the target cell with the immunoresponsive cell.

In certain embodiments, the status of the target cell is measured about 10 hours, about 15 hours, about 20 hours, about 24 hours, about 2 days, about 3 days, about 4 days, or about 5 days from the initial contact of the target cell with the immunoresponsive cell. In certain embodiments, the status of the target cell is measured about 20 hours from the initial contact of the target cell with the immunoresponsive cell. In certain embodiments, status of the target cell about 18 hours contact of the target cell is measured from the initial contact of the target cell with the immunoresponsive cell.

In certain embodiments, the status of the target cell is selected from the group consisting of cell death, cell proliferation, cell apoptosis, cell necrosis, cell autophagy, cell lysis, cell growth arrest, cell antigen expression suppression, cell chemokine receptor expression, cell chemokine secretion, cell receptor (e.g., PD-1, PD-2) expression, cell ligand (e.g., PD-L1, PD-L2) expression, and combinations thereof. In certain embodiments, the status of the target cell comprises cell death. In certain embodiments, the status of the target cell comprises cell apoptosis.

Any suitable methods for measuring the cell status can be used to measure the status of the target cell. In certain embodiments, the status of the target cell is measured by a chromium release assay, e.g., a Cr⁵¹ release assay. In certain embodiments, the status of the target cell is measured by a bioluminescence assay. In certain embodiments, the status of the target cell is measured by a flow cytometry assay. In certain embodiments, the status of the target cell is measured by an impedance assay, e.g., an xCELLigence system. In certain embodiments, the status of the target cell is measured by an apoptosis assay, an assay measuring chemokine secretion, an assay measuring cell ligand expression, an assay measuring cell receptor expression, or a combinations thereof.

In certain embodiments, the target cell comprises a tumor antigen or a pathogen antigen. Non-limiting examples of tumor antigens include mesothelin (MSLN), carbonic anhydrase IX (CA1X), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), ERBB2, MAGEA3, p53, MART1, GP100, Proteinase3 (PR1), Tyrosinase, Survivin, hTERT, EphA2, NKG2D ligands, cancer-testis antigen NY-ES0-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), and Wilms tumor protein (WT-1), BCMA, NKCS1, EGF1R, EGFR-VIII, CD99, CD70, ADGRE2, CCR1, LILRB2, PRAME and ERBB. In certain embodiments, the tumor antigen is mesothelin.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtherias, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

In certain embodiments, the target cell is a cancer cell. Non-limiting examples of cancer cells include lung cancer cells, breast cancer cells, ovarian cancer cells, leukemia cells, colorectal cancer cells, prostate cancer cells, sarcoma cells, mesothelioma cells, and lymphoma cells.

As used herein, an “immunotherapeutic agent” refers to an agent used in an immunotherapy. Immunotherapy is a treatment of a disease or disorder by activating or suppressing the immune system. Non-limiting examples of the immunotherapeutic agents include antibodies, immune checkpoint inhibitors, interferons, interferon alpha (e.g., Roferon-A, Intron A, Alferon), interleukins (e.g., IL-2), oncolytic virus (e.g., talimogene laherparepvec (Imlygic), T-VEC), and cancer vaccines, T cells, dendritic cells, bispecific antibodies, BiTEs, immunotoxins, and combinations thereof.

### 5.4. Immunoresponsive Cells

The immunoresponsive cells of the presently disclosed subject matter can be cells of the lymphoid lineage. The lymphoid lineage, comprising B cells, T cells, and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (Tregs, also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. In certain embodiments, the immunoresponsive cell is a T cell. The T cell can be a CD4⁺ T cell or a CD8⁺ T cell.

Natural killer (NK) cells can be lymphocytes that are part of cell-mediated immunity and act during the innate immune response. NK cells do not require prior activation in order to perform their cytotoxic effect on target cells.

In certain embodiments, the immunoresponsive cells comprise a receptor that binds to an antigen. In certain embodiments, the receptor is endogenous or exogenous. In certain embodiments, the receptor is a recombinant receptor. In certain embodiments, the antigen to which the receptor binds is the same as the antigen to which the target cell binds.

In certain embodiments, the antigen to which the receptor binds is a tumor antigen, e.g., one disclosed in Section 5.3. In certain embodiments, the antigen to which the receptor binds is a pathogen antigen, e.g., one disclosed in Section 5.3. In certain embodiments, the immunoresponsive cells comprise a receptor that binds to mesothelin.

In certain embodiments, the receptor is a T-cell receptor (TCR).

A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the receptor is a recombinant TCR. In certain embodiments, the is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

In certain embodiments, the receptor is a chimeric antigen receptor (CAR). CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., a scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40, CD27, CD40/My88 and NKGD2) to the cytoplasmic tail of the CAR to provide additional signals to the T cell. “Second generation” CARs comprise those that provide both co-stimulation (e.g., CD28 or 4-1BB) and activation (CD3). “Third generation” CARs comprise those that provide multiple co-stimulation (e.g., CD28 and 4-1BB) and activation (CD3). In certain embodiments, the CAR is a second-generation CAR. In certain embodiments, the CAR comprises an extracellular antigen-binding domain that binds to an antigen, a transmembrane domain, and an intracellular signaling domain, wherein the intracellular signaling domain comprises a co-stimulatory signaling domain. In certain embodiments, the CAR further comprises a hinger/spacer region.

In certain non-limiting embodiments, the extracellular antigen-binding domain of the CAR (embodied, for example, a scFv or an analog thereof) binds to the antigen with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸M or less, about 1×10⁻⁸M or less, about 9×10⁻⁹M or less, about 5×10⁻⁹M or less, about 4×10⁻⁹M or less, about 3×10⁻⁹ or less, about 2×10⁻⁹M or less, about 1×10⁻⁹M or less, about 1×10⁻¹⁰ M or less, or about 1×10⁻¹¹M or less. In certain non-limiting embodiments, the K_{d} is about 1×10⁻⁸M or less. In certain non-limiting embodiments, the K_{d} is about 1×10⁻⁹M or less. In certain non-limiting embodiments, the K_{d} is from about 1×10⁻⁹M to about 1×10⁻⁷ M.

Binding of the extracellular antigen-binding domain (for example, in a scFv or an analog thereof) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

In certain embodiments, the extracellular antigen-binding domain of the CAR comprises a scFv (e.g., a murine, human, or humanized scFv), a Fab (which is optionally crosslinked), or a F(ab)₂. In certain embodiments, any of the foregoing molecules may be comprised in a fusion protein with a heterologous sequence to form the extracellular antigen-binding domain.

In certain embodiments, the extracellular antigen-binding domain of the CAR comprises a heavy chain variable region CDR1 comprising amino acids having the sequence set forth in SEQ ID NO:1 or conservative modifications thereof, a heavy chain variable region CDR2 comprising amino acids having the sequence set forth in SEQ ID NO:2 or conservative modifications thereof, and a heavy chain variable region CDR3 comprising amino acids having the sequence set forth in SEQ ID NO:3 or conservative modifications thereof. In certain embodiments, the extracellular antigen-binding domain of the CAR comprises a light chain variable region CDR1 comprising amino acids having the sequence set forth in SEQ ID NO:4 or conservative modifications thereof, a light chain variable region CDR2 comprising amino acids having the sequence set forth in SEQ ID NO:5 or conservative modifications thereof, and a light chain variable region CDR3 comprising amino acids having the sequence set forth in SEQ ID NO:6 or conservative modifications thereof. SEQ ID NOs: 1-6 are provided below:

[SEQ ID NO: 1]
GGSVSSGSYY
[SEQ ID NO: 2]
IYYSGST
[SEQ ID NO: 3]
AREGKNGAFDIW
[SEQ ID NO: 4]
QSISSY
[SEQ ID NO: 5]
AASS
[SEQ ID NO: 6]
QQSYSTPLTF

In certain embodiments, the extracellular antigen-binding domain of the CAR comprises a V_{H} CDR1 comprising amino acids having the sequence set forth in SEQ ID NO: 1, a V_{H} CDR2 comprising the amino acid sequence set forth in SEQ ID NO: 2, a V_{H} CDR3 comprising the amino acid sequence set forth in SEQ ID NO: 3, a V_{L} CDR1 comprising the amino acid sequence set forth in SEQ ID NO: 4, a V_{L} CDR2 comprising the amino acid sequence set forth in SEQ ID NO: 5, and a V_{L} CDR3 comprising the amino acid sequence set forth in SEQ ID NO: 6. In certain embodiments, the CDRs are identified according to the Kabat numbering system.

As used herein, the term “conservative sequence modifications” refers to amino acid modifications that do not significantly affect or alter the binding characteristics of the presently disclosed CAR (e.g., the extracellular antigen-binding domain) comprising the amino acid sequence. Such conservative modifications include amino acid substitutions, additions and deletions. Modifications can be introduced into the human scFv of the presently disclosed subject matter by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered. In certain embodiments, the immunoresponsive cells comprise a CAR that binds to mesothelin. In certain embodiments, the extracellular antigen-binding domain of the CAR (e.g., a scFv) comprises a heavy chain variable region (V_{H}) comprising the amino acid sequence set forth in SEQ ID NO: 7. In certain embodiments, the extracellular antigen-binding domain of the CAR (e.g., a scFv) comprises a light chain variable region (V_{L}) comprising the amino acid sequence set forth in SEQ ID NO: 8. In certain embodiments, the extracellular antigen-binding domain of the CAR (e.g., a scFv) comprises a V_{H} comprising the amino acid sequence set forth in SEQ ID NO: 7 and a V_{L} comprising the amino acid sequence set forth in SEQ ID NO: 8, optionally with (iii) a linker sequence, for example a linker peptide, between the V_{H} and the V_{L}. In certain embodiments, the linker comprises amino acids having the sequence set forth in SEQ ID NO: 9. In certain embodiments, the extracellular antigen-binding domain of the CAR (e.g., a scFv) comprises a V_{H} comprising an amino acid sequence that is at least about 80% (e.g., at least about 85%, at least about 90%, or at least about 95%) homologous or identical to SEQ ID NO: 7. For example, the extracellular antigen-binding domain of the CAR (e.g., a scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 7. In certain embodiments, the extracellular antigen-binding domain comprises a V_{H} comprising the amino sequence set forth in SEQ ID NO: 7. In certain embodiments, the extracellular antigen-binding domain of the CAR (e.g., a scFv) comprises a V_{L} comprising an amino acid sequence that is at least about 80% (e.g., at least about 85%, at least about 90%, or at least about 95%) homologous or identical to SEQ ID NO: 8. For example, the extracellular antigen-binding domain of the CAR (e.g., a scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 8. In certain embodiments, the extracellular antigen-binding domain of the CAR (e.g., a scFv) comprises a V_{L} comprising the amino acid sequence set forth in SEQ ID NO: 8. In certain embodiments, the extracellular antigen-binding domain of the CAR (e.g., a scFv) comprises a V_{H} comprising an amino acid sequence that is at least about 80% (e.g., at least about 85%, at least about 90%, or at least about 95%) homologous or identical to SEQ ID NO: 7, and a V_{L} comprising an amino acid sequence that is at least about 80% (e.g., at least about 85%, at least about 90%, or at least about 95%) homologous or identical to SEQ ID NO: 8. In certain embodiments, the extracellular antigen-binding domain of the CAR (e.g., a scFv) comprises a V_{H} comprising the amino acid sequence set forth in SEQ ID NO: 7 and a V_{L} comprising the amino acid sequence set forth in SEQ ID NO: 8.

SEQ ID NOS: 7-9 are provided below.

[SEQ ID NO: 7]
QVQLQESGPGLVKPSETLSLTCTVSGGSVSSGSYYWSWIRQPPGKGLEWIG
YIYYSGSTNYNPSLKSRVTISVDTSKNQFSLKLSSVTAADTAVYYCAREGK
NGAFDIWGQGTMVTVSS
[SEQ ID NO: 8]
RHQMTQSPSSLSASVGDRVTITCRASQSISSYLNWYQQKPGKAPKLLIYAA
SSLQSGVPSRFSGSGSGTDFTLTISSLQPEDFATYYCQQSYSTPLTEGGGT
KVEIKRT
[SEQ ID NO: 9]
GGGGSGGGGSGGGGS.

An exemplary nucleic acid sequence encoding the amino acid sequence of SEQ ID NO: 7 is set forth in SEQ ID NO:10, which is provided below.

[SEQ ID NO: 10]
CAGGTTCAGCTTCAGGAGAGTGGCCCAGGCCTGGTGAAGCCAAGTGAGACT
CTCAGCTTGACTTGCACAGTTTCTGGAGGCAGTGTCTCCTCAGGCAGCTAT
TATTGGTCCTGGATTCGGCAGCCCCCTGGGAAAGGCCTGGAGTGGATTGGG
TACATATATTACAGTGGCAGCACAAATTACAATCCATCCCTGAAGTCTCGA
GTAACTATCAGTGTGGACACAAGCAAGAATCAGTTTTCACTCAAACTGTCT
TCTGTGACTGCTGCTGACACTGCTGTTTATTATTGTGCCAGGGAGGGGAAA
AATGGGGCATTTGATATTTGGGGTCAGGGCACAATGGTGACAGTCAGCTCT

An exemplary nucleic acid sequence encoding the amino acid sequence of SEQ ID NO:8 is set forth in SEQ ID NO:11, which is provided below.

[SEQ ID NO: 11]
CGCCATCAGATGACTCAGTCCCCCTCCAGTCTTTCTGCCTCAGTTGGGGAT
AGAGTGACCATCACATGCAGAGCAAGTCAGAGCATATCATCCTATCTGAAC
TGGTACCAGCAGAAGCCAGGGAAAGCCCCCAAATTGCTGATTTATGCAGCC
TCAAGTCTCCAGAGTGGGGTGCCAAGCAGGTTCTCAGGCAGTGGCAGTGGG
ACAGATTTCACATTGACAATCAGCTCCCTCCAACCTGAAGATTTTGCCACC
TACTATTGCCAGCAATCCTACAGCACGCCCCTGACTTTTGGAGGTGGCACA
AAGGTAGAGATCAAGAGGACT

In certain non-limiting embodiments, the intracellular signaling domain of the CAR comprises a CD3ζ polypeptide, which can activate or stimulate a cell (e.g., a cell of the lymphoid lineage, e.g., a T cell). In certain embodiments, the intracellular signaling domain of the CAR comprises a CD3ζ polypeptide comprising or having amino acids 52 to 164 of SEQ ID NO: 12. SEQ ID NO: 12 is provided below:

[SEQ ID NO: 12]

| | |
|---|---|
| 1 | MKWKALFTAA ILQAQLPITE AQSFGLLDPK LCYLLDGILF |
| | IYGVILTALF LRVKFSRSAD |
| 61 | APAYQQGQNQ LYNELNLGRR EEYDVLDKRR GRDPEMGGKP |
| | QRRKNPQEGL YNELQKDKMA |
| 121 | EAYSEIGMKG ERRRGKGHDG LYQGLSTATK DTYDALHMQA |
| | LPPR |

An exemplary nucleic acid sequence encoding amino acids 52 to 164 of SEQ ID NO: 12 is set forth in SEQ ID NO:13, which is provided below.

[SEQ ID NO: 13]
AGAGTGAAGTTCAGCAGGAGCGCAGACGCCCCCGCGTACCAGCAGGGCCAG
AACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGG
AAGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGGG
CACGATGGCCTTTACCAGGGTCTCAGTACAGCCACCAAGGACACCTACGAC
GCCCTTCACATGCAGGCCCTGCCCCCTCGCTAA

In certain non-limiting embodiments, the intracellular signaling domain of the CAR further comprises at least a co-stimulatory signaling region. In certain embodiments, the co-stimulatory signaling region comprises at least one co-stimulatory molecule, which can provide optimal lymphocyte activation.

As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, a CD27 polypeptide, a CD40/My88 polypeptide, a NKGD2 polypeptide or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR′ T cell. CARs comprising an intracellular signaling domain that comprises a co-stimulatory signaling region comprising 4-1BB, ICOS or DAP-10 are disclosed in U.S. Pat. No. 7,446,190, which is herein incorporated by reference in its entirety.

In certain embodiments, the intracellular signaling domain of the CAR comprises a co-stimulatory signaling region that comprises a CD28 polypeptide. In certain embodiments, the intracellular signaling domain of the CAR comprises the intracellular signaling domain of a human CD28 polypeptide. In certain embodiments, the intracellular signaling domain of a human CD28 polypeptide comprises or has an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 101 or fragments thereof, and/or may optionally comprise up to one or up to two or up to three conservative amino acid substitutions. In certain embodiments, the intracellular signaling domain of a CD28 polypeptide comprises or has the amino acid sequence set forth in SEQ ID NO: 14. SEQ ID NO: 14 is provided below:

[SEQ ID NO: 14]
RSKRSRLLHS DYMNMTPRRP GPTRKHYQPY APPRDFAAYR S

An exemplary nucleic acid sequence encoding the amino acid sequence of SEQ ID NO: 14 is set forth in SEQ ID NO: 15, which is provided below.

[SEQ ID NO: 15]
AGGAGTAAGAGGAGCAGGCTCCTGCACAGTGACTACATGAACATGACTCCC
CGCCGCCCCGGGCCCACCCGCAAGCATTACCAGCCCTATGCCCCACCACGC
GACTTCGCAGCCTATCGCTCC

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In certain non-limiting embodiments, the transmembrane domain of the CAR comprises a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, a synthetic polypeptide (not based on a protein associated with the immune response), or a combination thereof.

In certain embodiments, the transmembrane domain of the CAR comprises the transmembrane domain of a CD28 polypeptide. In certain embodiments, the transmembrane domain of the CAR comprises the transmembrane domain of a human CD28 polypeptide. In certain embodiments, the transmembrane domain a human CD28 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to SEQ ID NO: 16 or fragments thereof, and/or may optionally comprise up to one or up to two or up to three conservative amino acid substitutions. In certain embodiments, the transmembrane domain a human CD28 polypeptide comprises or has the amino acid sequence set forth in SEQ ID NO: 16. SEQ ID NO: 16 is provided below:

[SEQ ID NO: 16]
FWVLVVVGGV LACYSLLVTV AFIIFWV.

An exemplary nucleic acid sequence encoding the amino acid sequence of SEQ ID NO: 16 is set forth in SEQ ID NO: 17, which is provided below.

[SEQ ID NO: 17]
TTTTGGGTGCTGGTGGTGGTTGGTGGAGTCCTGGCTTGCTATAGCTTGCTA
GTAACAGTGGCCTTTATTATTTTCTGGGTG

In certain embodiments, the immunoresponsive cells comprise a mesothelin-targeted CAR disclosed in WO 2015/188141, which is incorporated by reference hereby in its entirety.

In certain embodiments, the immunoresponsive cells comprise a mesothelin-targeted CAR “M28z”, which comprises (a) an extracellular antigen-binding domain comprising a V_{H} CDR1 comprising amino acids having the sequence set forth in SEQ ID NO: 1, a V_{H} CDR2 comprising the amino acid sequence set forth in SEQ ID NO: 2, a V_{H} CDR3 comprising the amino acid sequence set forth in SEQ ID NO: 3, a V_{L} CDR1 comprising the amino acid sequence set forth in SEQ ID NO: 4, a V_{L} CDR2 comprising the amino acid sequence set forth in SEQ ID NO: 5, and a V_{L} CDR3 comprising the amino acid sequence set forth in SEQ ID NO: 6; (b) a transmembrane domain comprising a CD28 polypeptide having the amino acid sequence set forth in SEQ ID NO: 16; (c) an intracellular signaling domain comprising (i) a CD3ζ polypeptide having amino acids 52 to 164 of SEQ ID NO: 12; and (ii) a co-stimulatory signaling region comprising a CD28 polypeptide having the amino acid sequence set forth in SEQ ID NO: 14.

### 5.5. Kits and Systems

The present disclosure further provides kits and systems for assessing cytotoxicity of immunoresponsive cells. In certain embodiments, the kit or system comprises a pleural effusion as described by the present disclosure (e.g., as disclosed in Section 5.2), a target cell as described by the present disclosure (e.g., as disclosed in Section 5.3), and an immunoresponsive cell as described by the present disclosure (e.g., as disclosed in Section 5.4).

In certain embodiments, the kit or system further comprises instructions for assessing cytotoxicity of immunoresponsive cells. In certain embodiments, the instructions comprise methods for assessing cytotoxicity of immunoresponsive cells as described by the present disclosure (e.g., as disclosed in Section 5.3).

The present disclosure also provides kits and systems for assessing the effect of an immunotherapeutic agent on cytotoxicity of immunoresponsive cells. In certain embodiments, the kit or system comprises a pleural effusion as described by the present disclosure (e.g., as disclosed in Section 5.2), a target cell as described by the present disclosure (e.g., as disclosed in Section 5.3), an immunotherapeutic agent as described by the present disclosure (e.g., as disclosed in Section 5.3), and an immunoresponsive cell as described by the present disclosure (e.g., as disclosed in Section 5.4).

In certain embodiments, the kit or system further comprises instructions for assessing the effect of an immunotherapeutic agent on cytotoxicity of immunoresponsive cells. In certain embodiments, the instructions comprise methods for assessing the effect of an immunotherapeutic agent on cytotoxicity of immunoresponsive cells as described by methods of the present disclosure (e.g., as disclosed in Section 5.3).

### 6. EXAMPLES

### Example 1: An Immunocompetent Ex-Vivo Pleural Effusion Culture System

Human immunotherapeutic agent investigation to date is typically conducted in vitro with addition of one target or immune cell in media, and in vivo in immunodeficient mice prior to conducting safety studies in primates and translating into early-phase clinical trials. In the ex-vivo pleural effusion culture system, addition of one or more immune and/or target cells along with addition of one or more effectors and a mixture of cytokines/chemokines allows investigation of human immunotherapeutic agents in a complex microenvironment. Such a complex investigation pre-necessitates optimization of each component prior to administrating the mixture. This Example shows such optimization, followed by investigation of CAR T cells.

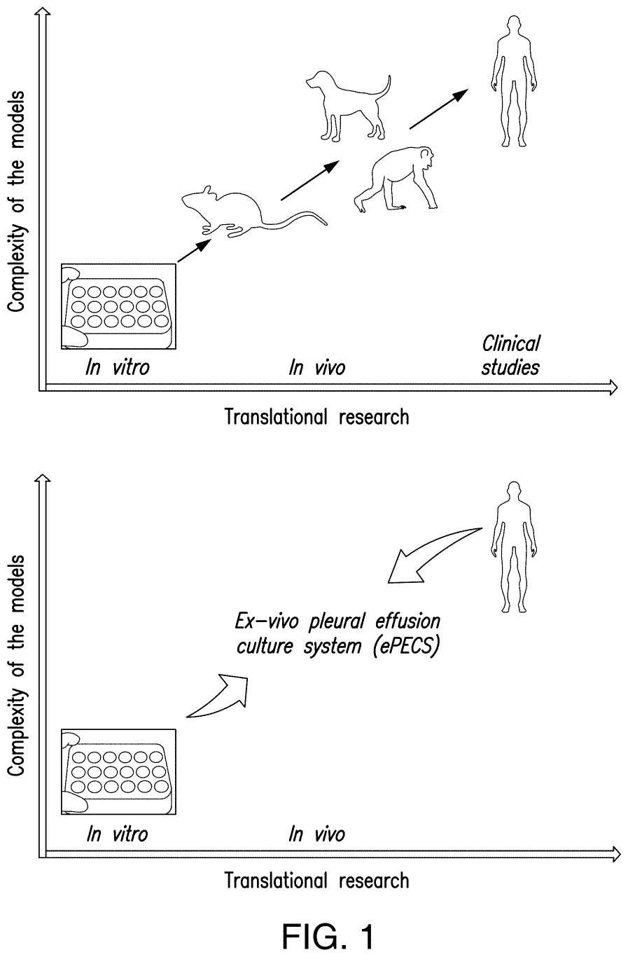

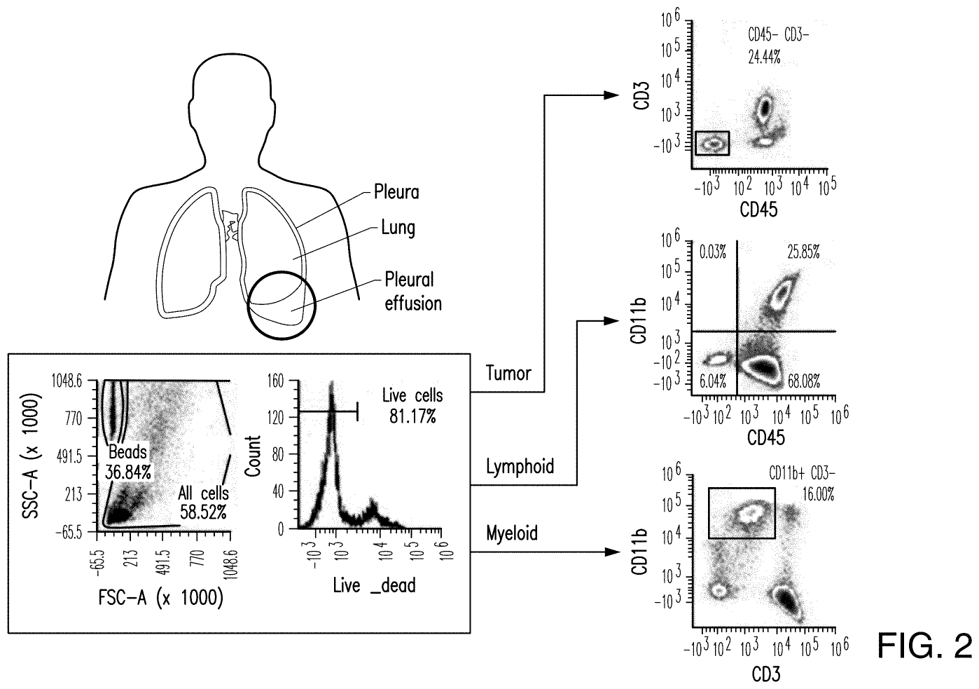

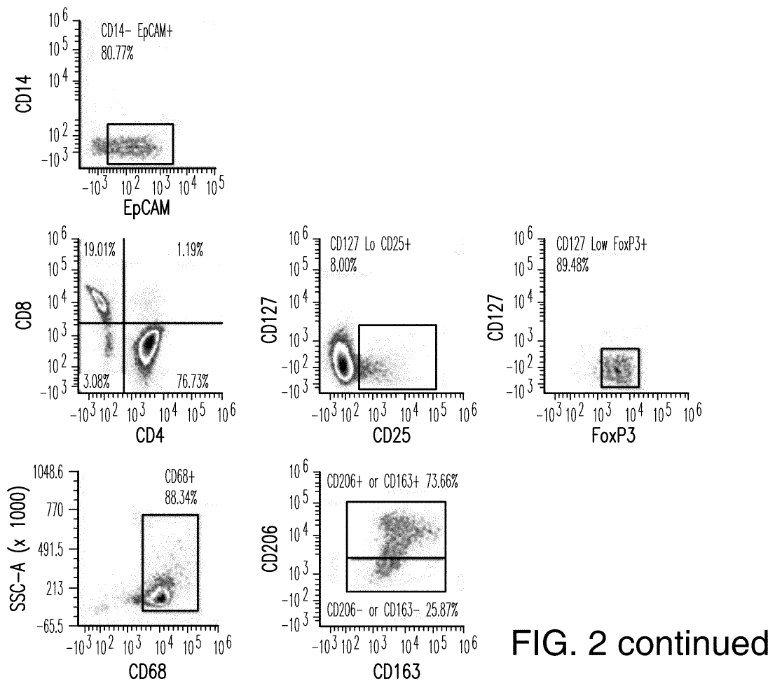

Methods and systems disclosed herein, e.g., ex-vivo plural effusion culture system (ePECS) can be used to replace the available assays for translational research, and to fulfill unmet need for non-existing human model system to investigate immunotherapeutic agents (FIG. 1). Cell components of the malignant plural effusion (MPE) obtained from patients include tumor cells, and a full complement of immune cells including T cells, Tregs, B cells, NK cells, neutrophils, macrophages, and dendritic cells, and cytokines (FIGS. 2-3).

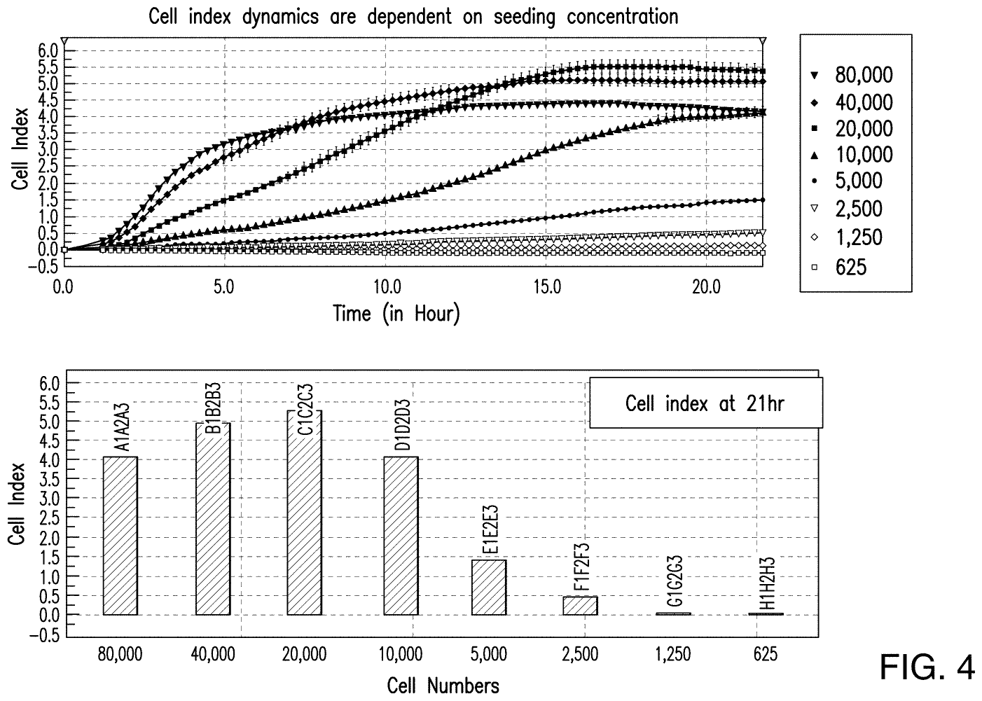

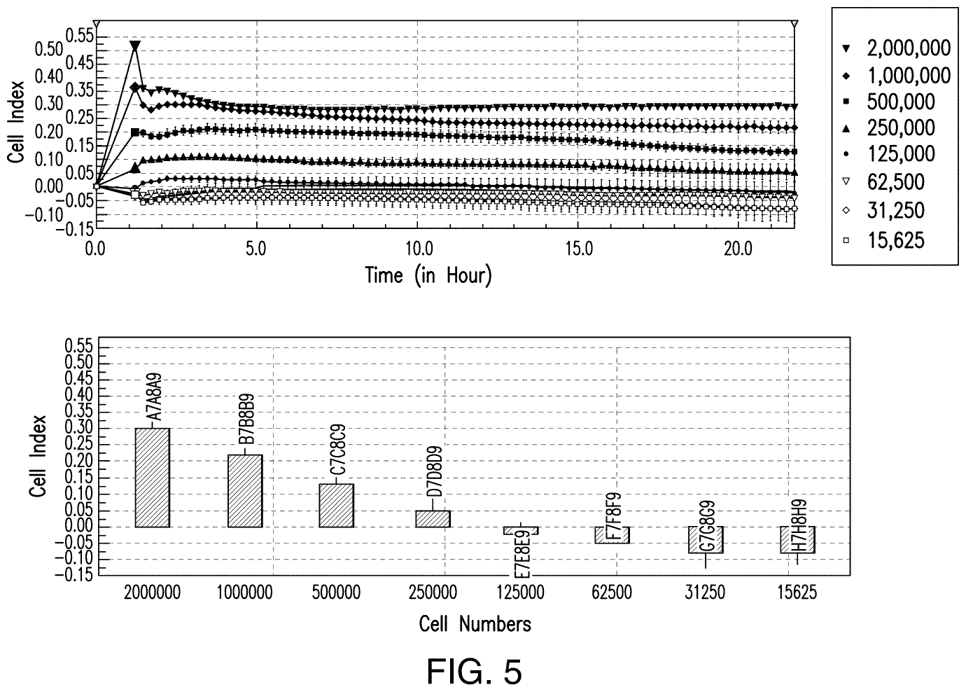

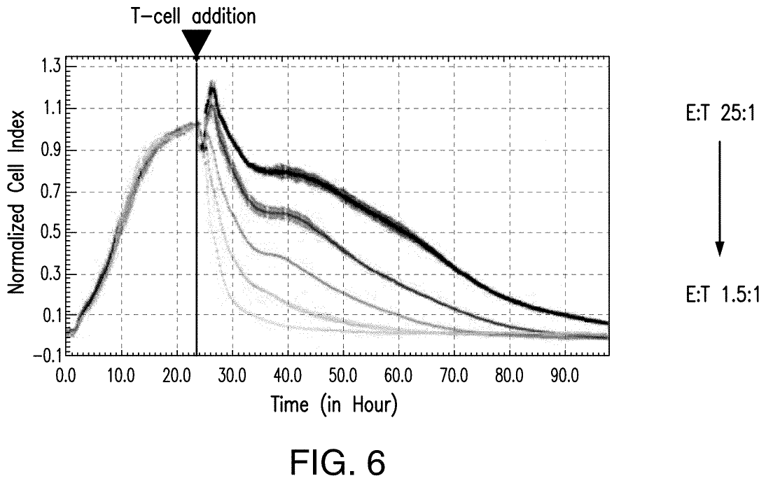

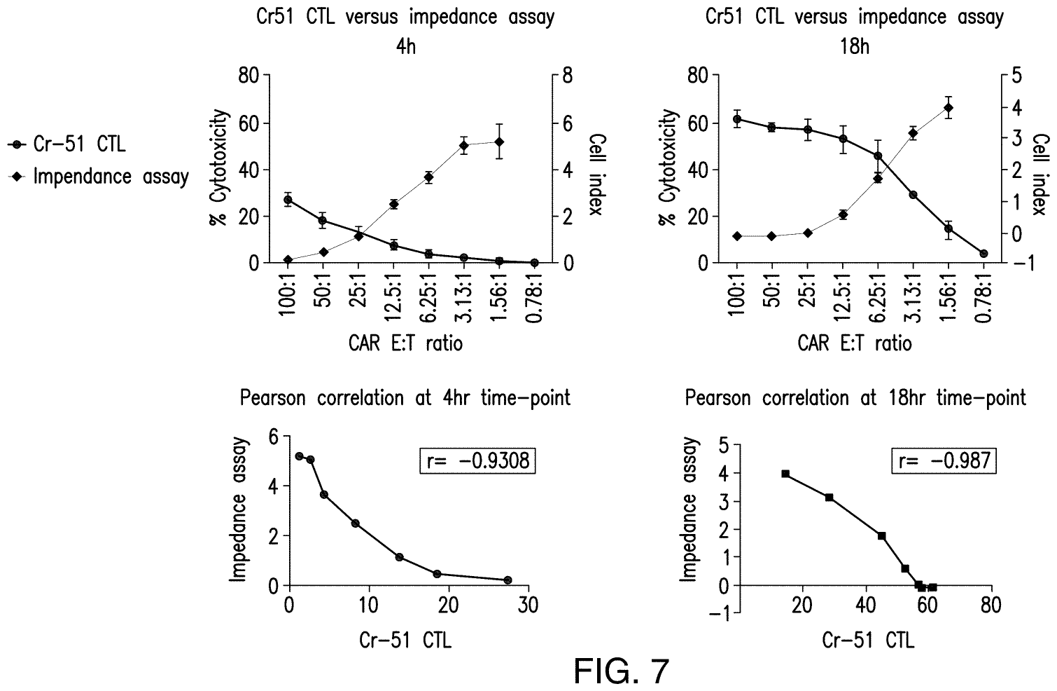

Impedance assay conditions were optimized. The optimal seeding concentration of tumor cells for the impedance assay was between 10,000-20,000 cells/well (FIG. 4). T cells had a concentration-dependent but minimal effect on impedance (FIG. 5). Impact of T cells at low E:T ratios on cell index was negligible. CAR T-cell cytotoxicity demonstrated a concentration-dependent drop in cell index (FIG. 6). The present Example shows that the impedance assay was comparable to (equally effective as) Chromium (Cr) release cytotoxicity T lymphocyte (CTL) assay (FIG. 7).

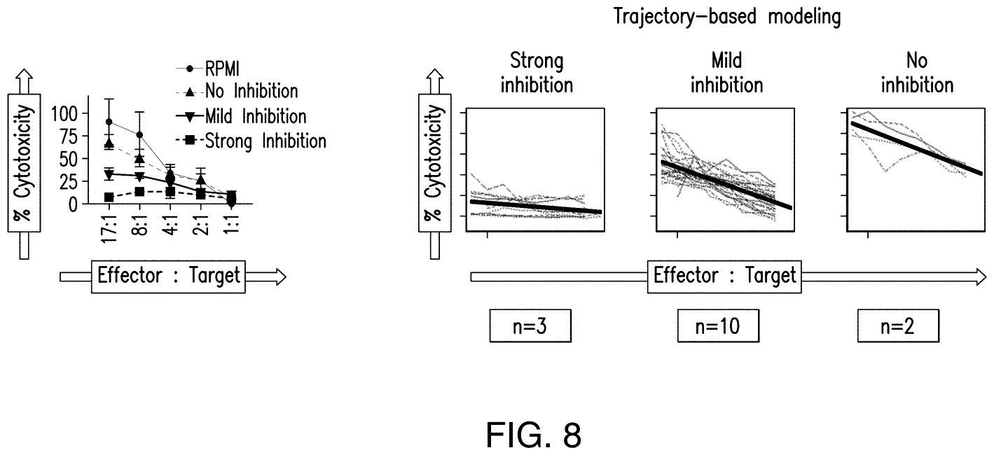

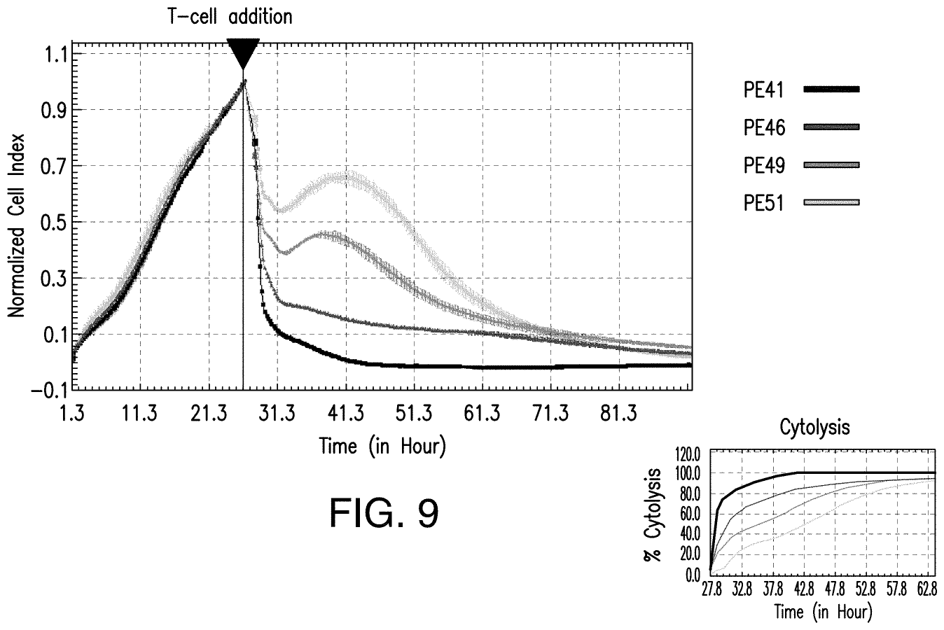

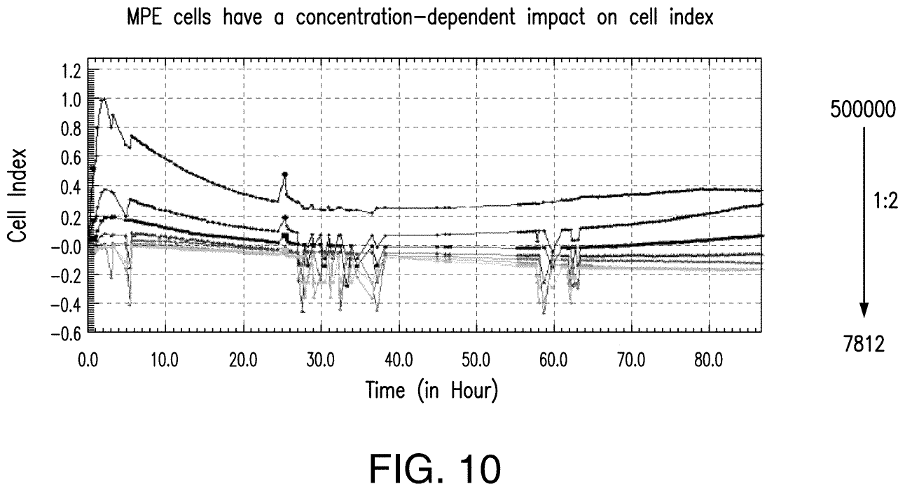

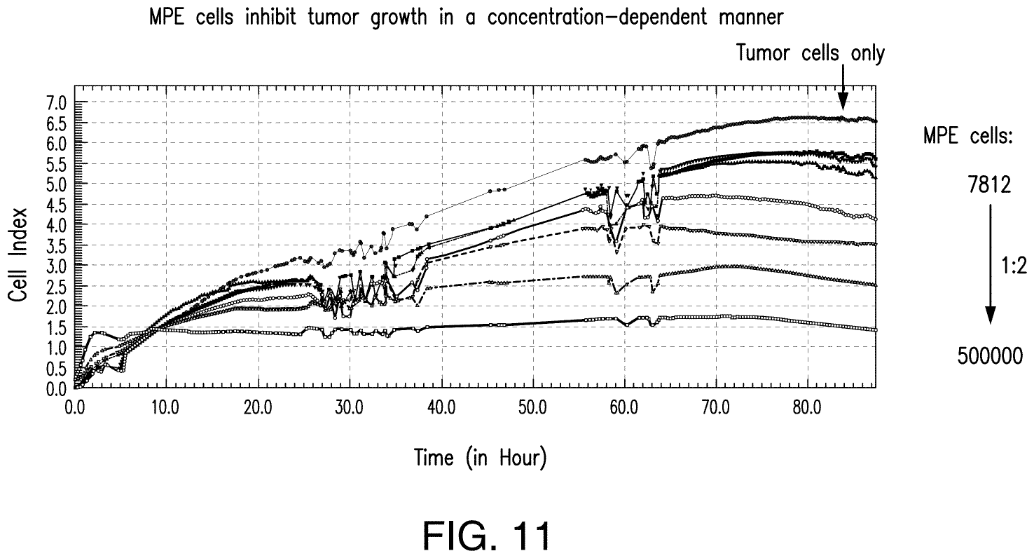

Cell-free pleural effusions influenced CAR T-cell efficacy (FIG. 8). CAR T cells obtained from different donors were influenced differently by pleural effusions from different patients. CAR T cells cultured in a cell-free pleural effusion from different patients had differential cytotoxicity (FIG. 9). MPE cells had a concentration-dependent impact on cell index (FIG. 10) and tumor growth (FIG. 11).

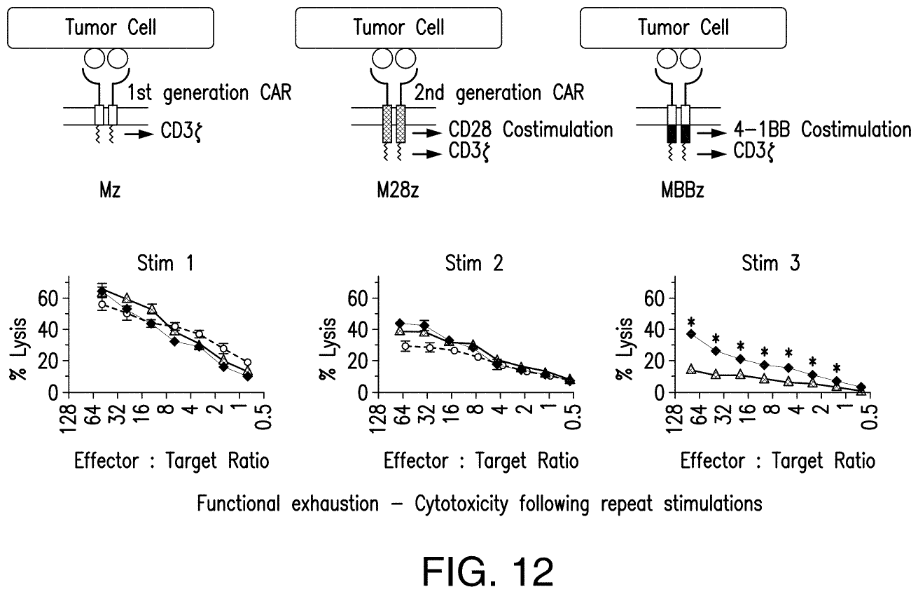

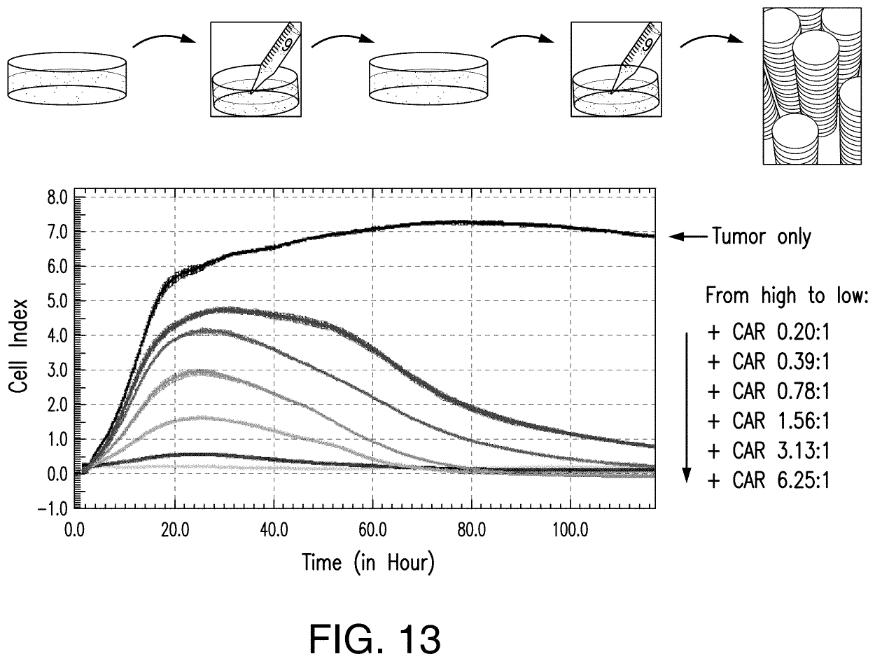

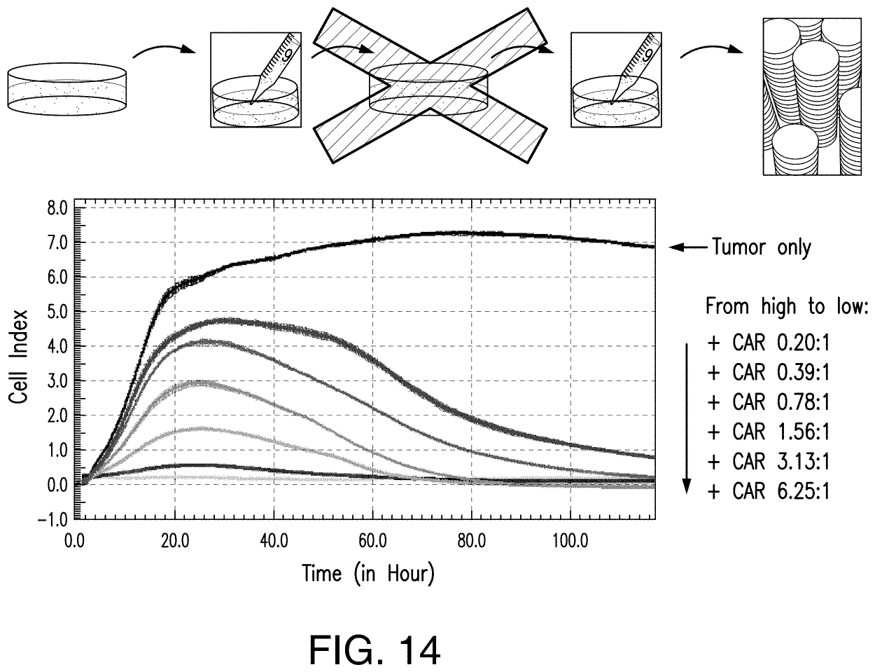

CAR T cells following repeat stimulations had functional exhaustion relating to cytotoxicity (FIG. 12). CAR T-cell cytotoxicity following exposure to repeat antigen stimulation were measured using antigen stress test for CAR T-cells (FIGS. 13-14).

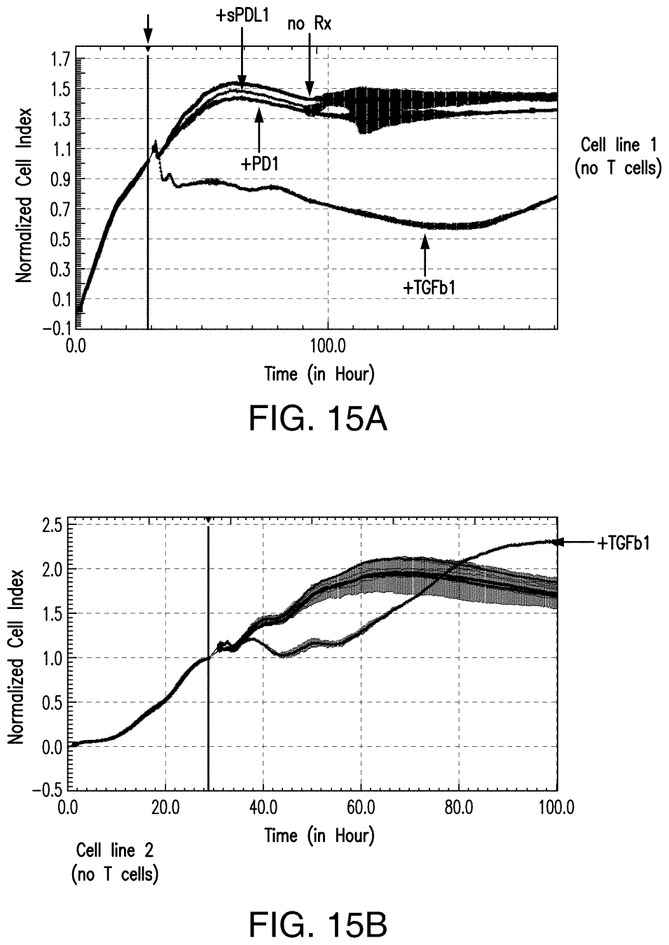

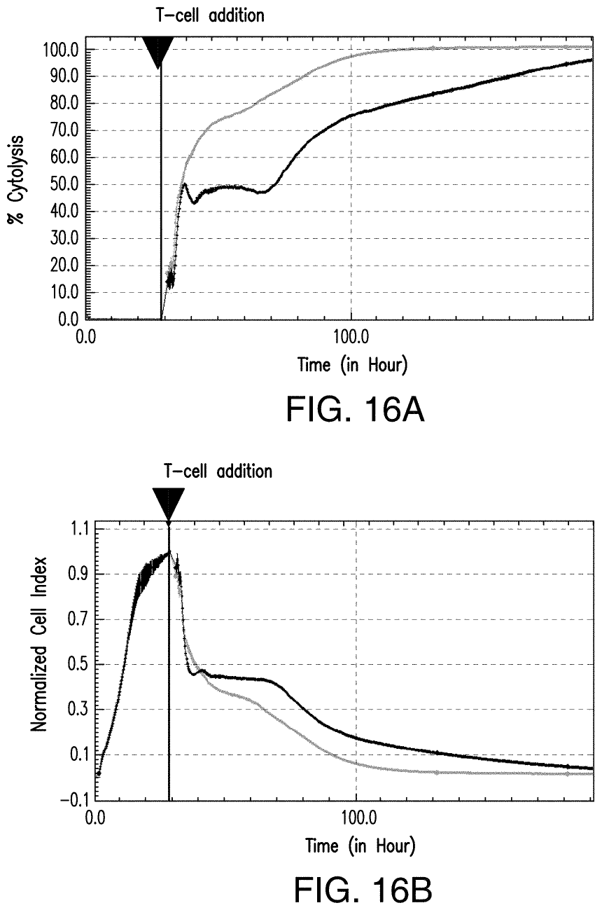

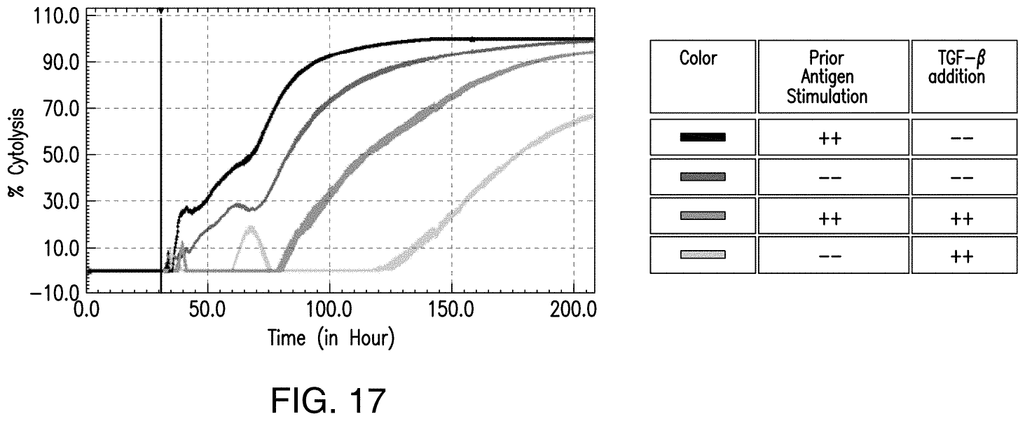

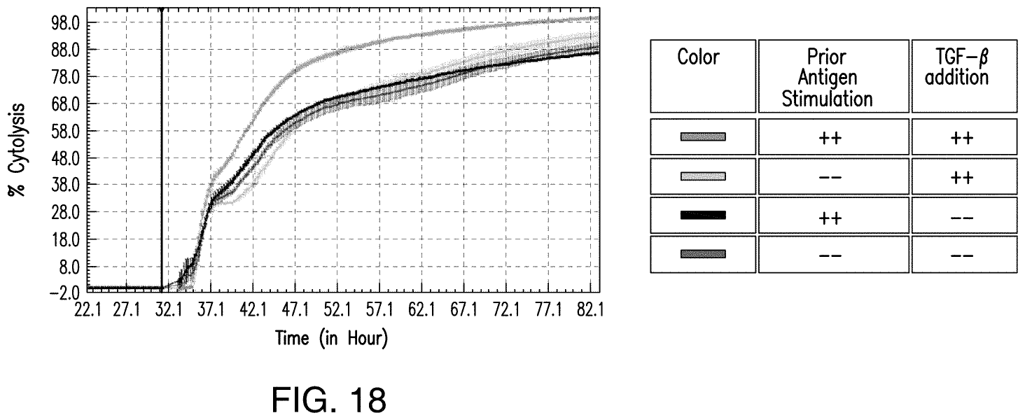

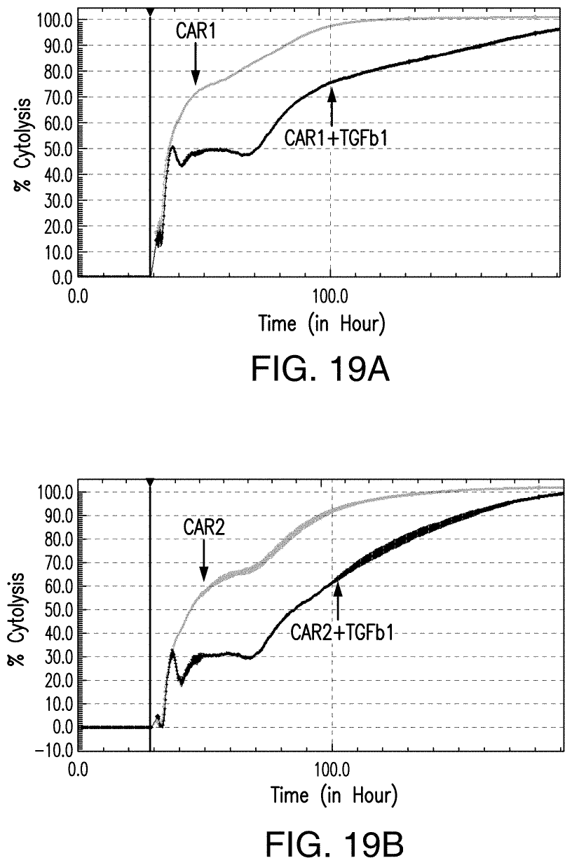

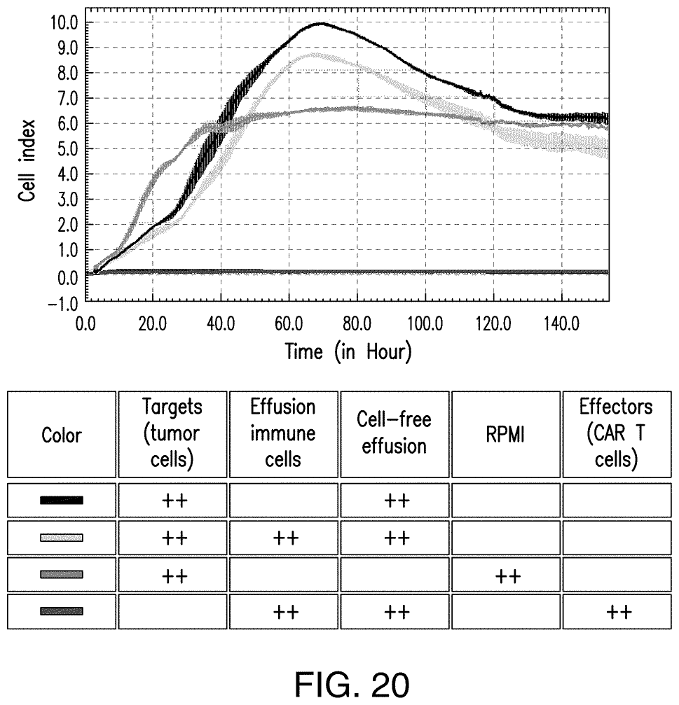

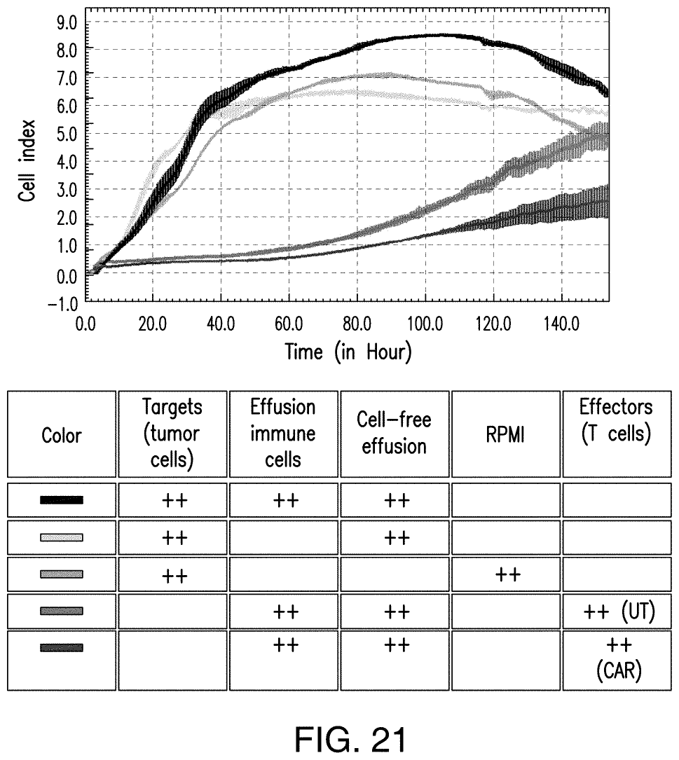

The influence of TGF-β on tumor growth was measured in cell line 1 (FIG. 15A) and cell line 2 (FIG. 15B). Recombinant TGF-β inhibited CAR T-cell efficacy as measured by cytolysis (FIG. 16A) and cell index (FIG. 16B). TGF-β inhibition depended on activation status of CAR T cells and the prior antigen stimulation of CAR T cells (FIG. 17). Cytotoxicity kinetics of CAR T-cells in the presence of TGF-β, and the rescue of CAR T-cell cytotoxicity in the presence of anti-TGFβ antibody were measured (FIG. 18). CAR T-cell 1 and CAR T-cell 2 were equally inhibited by TGF-β (FIG. 19). The impact of pleural effusion cells and cell-free effusion on target and CAR T cells were evaluated and represented as cell index (FIGS. 20-21).

### Example 2: MPEs Differentially Affect CAR T-Cell Cytotoxicity

Even cell-free MPEs, due to their constituent cytokines/chemokines influence T-cell cytotoxicity differentially. In this example, CAR T cells from multiple donors were incubated with MPEs obtained from different patients exhibiting differential cytotoxicity and chemokine expression.

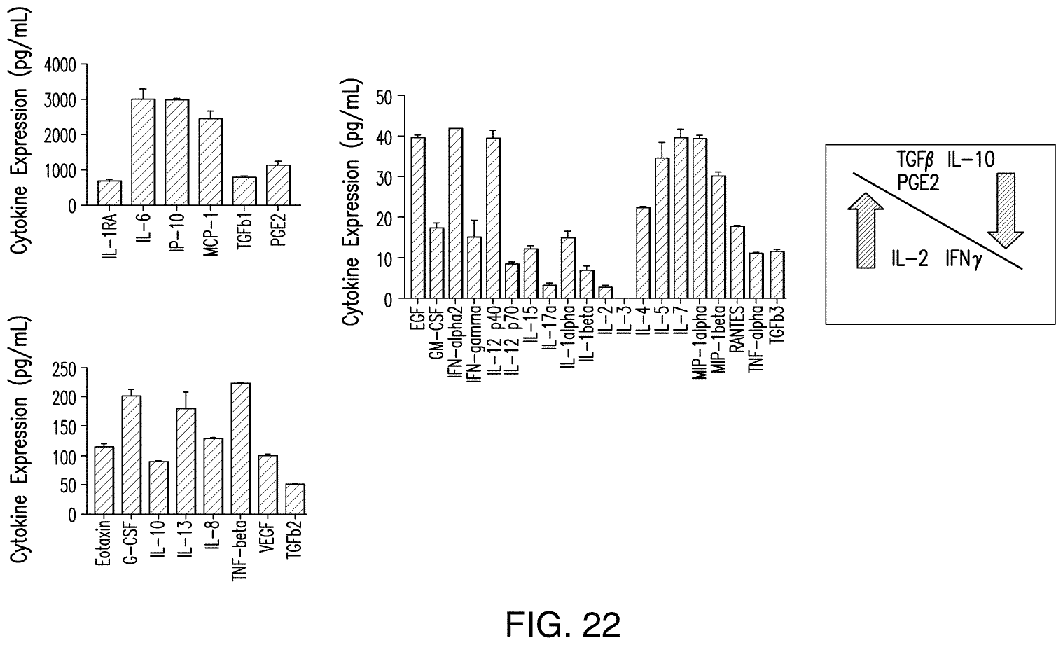

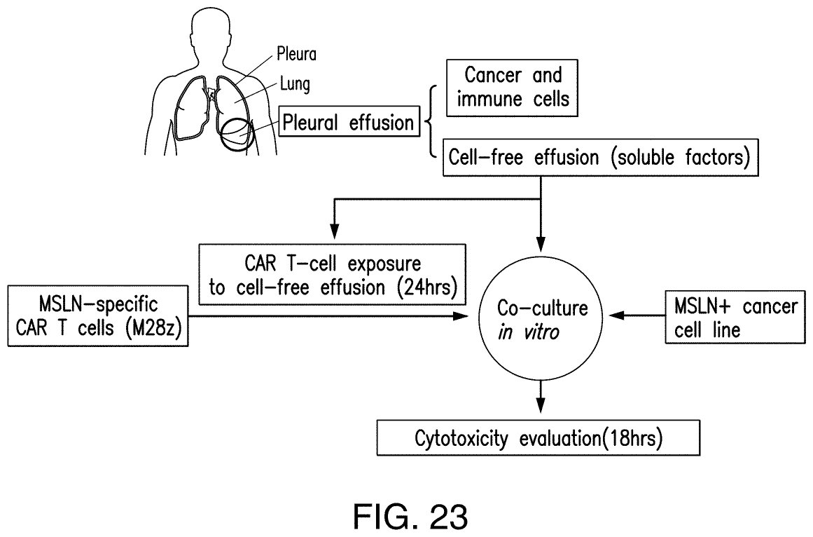

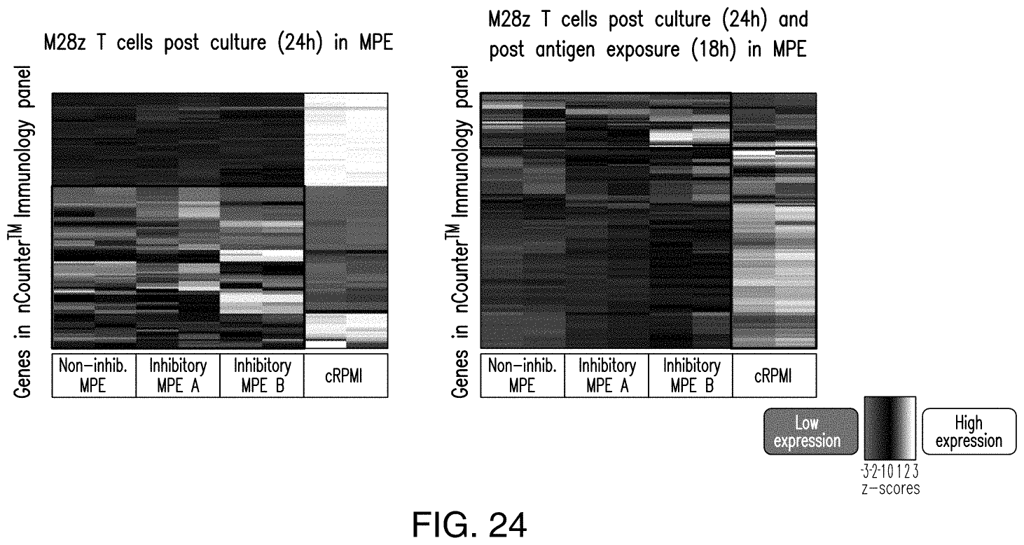

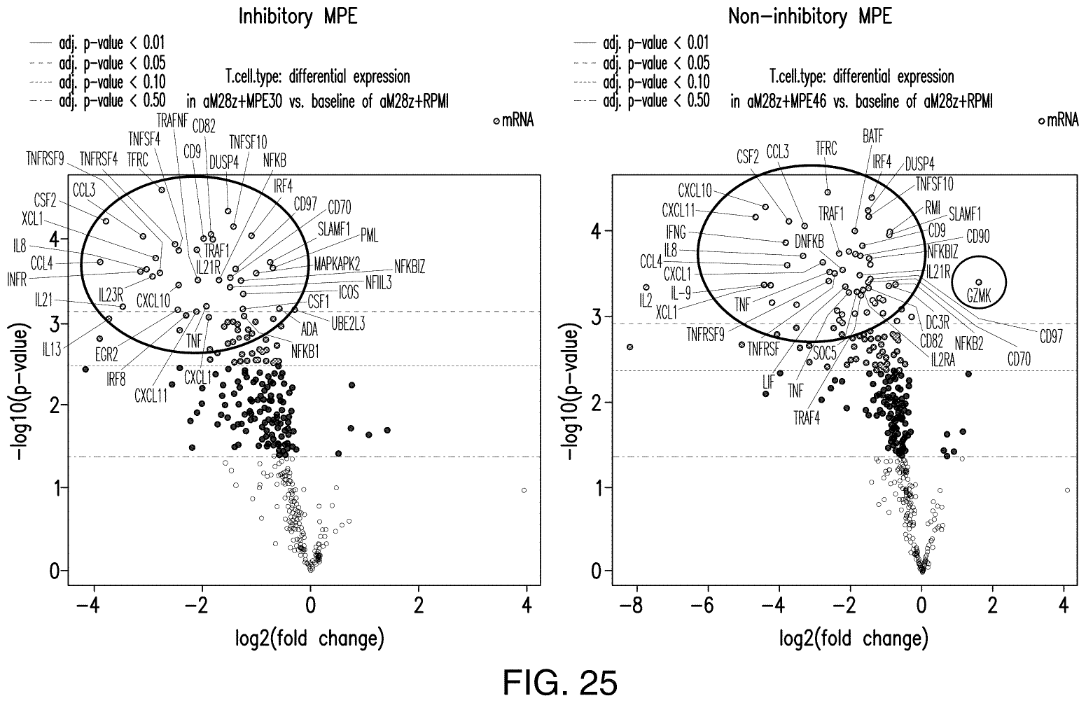

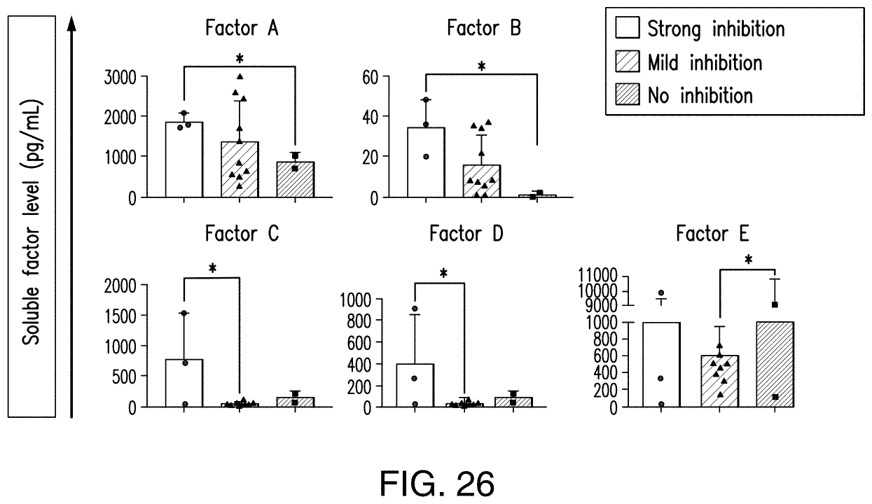

The composite of MPE includes immune and tumor cell-derived soluble factors (FIG. 22). An exemplary scheme of the methods and systems for evaluating the activity of antigen-receptors in accordance with certain embodiments of the present disclosure was shown in FIG. 23. Cytotoxicity was evaluated at 18 hours post-co-culture. M28z T cell 24 hour post-culture in MPE were treated with or without antigen exposure, and RNA were purified for gene expression analysis. Gene expression of CAR T cells varied upon exposure to MPE (FIG. 24). MPE induced downregulation of effector gene expression (FIG. 25). CAR T-cell cytotoxicity was associated with soluble factors present in MPE (FIG. 26).

### Example 3: Impact of Soluble PD-1, PD-L1 and TGFβ1 on M28z and M28z-PD1DNR-Mediated Cytotoxicity Towards A549GM and MGM

Effector T cells such as CAR T cells are influenced by external factors such as cytokines depending upon their genetic constitution. For example, although PD-L1 and TGF-beta can influence T cell cytotoxicity, the magnitude of influence or inhibition depends upon the genetic constitution of CAR within the T cell as well as the composition and dose of inhibitory cytokine or ligand within the MPE.

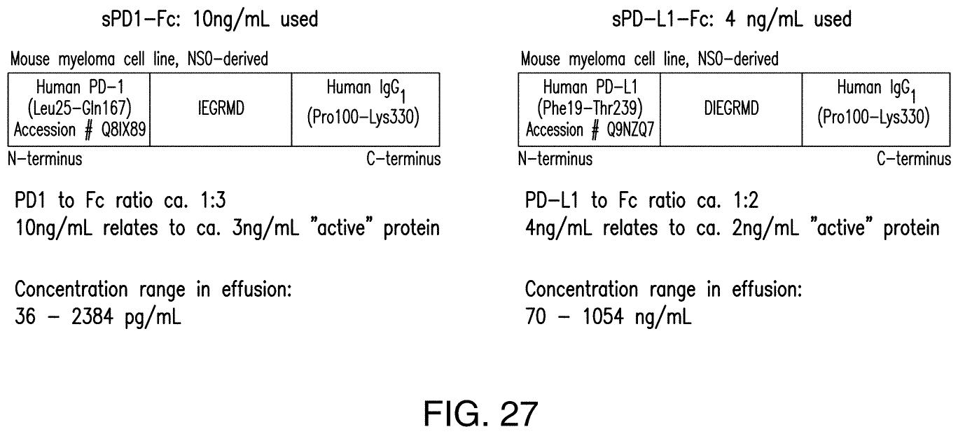

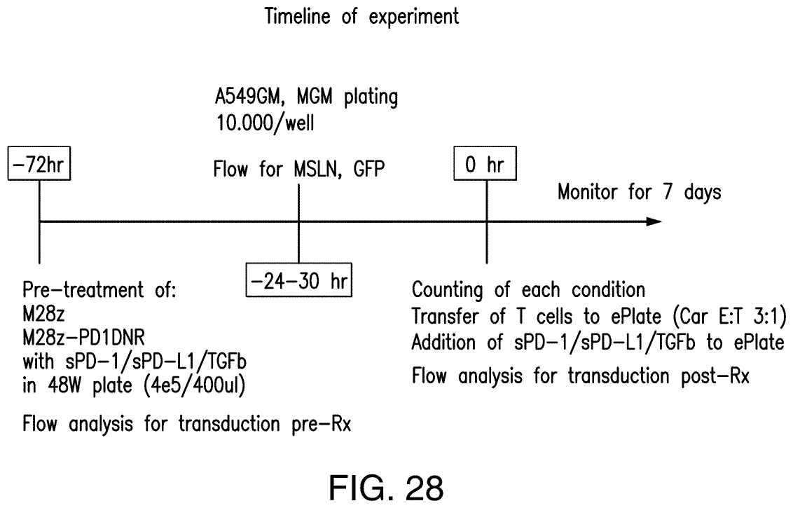

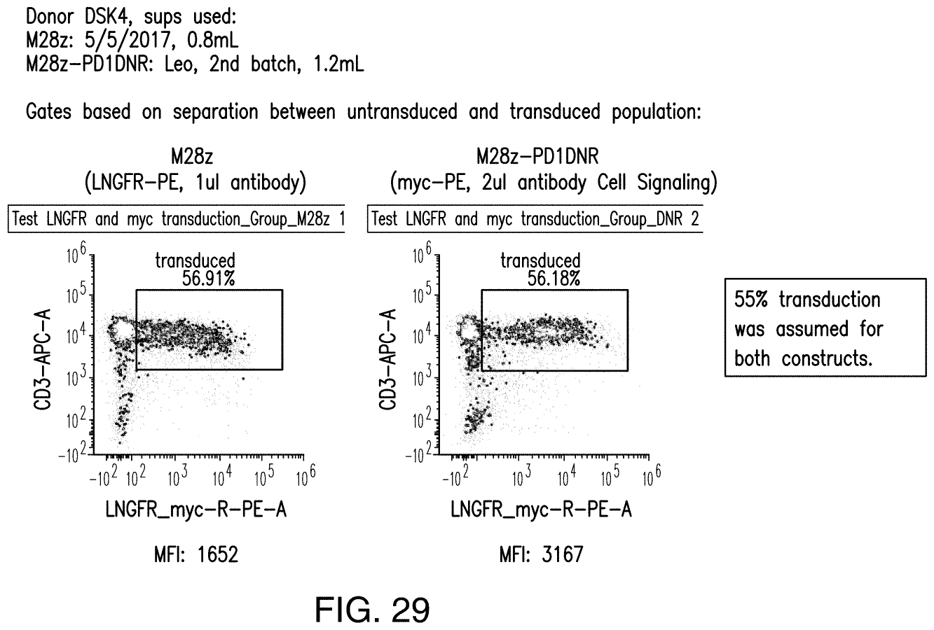

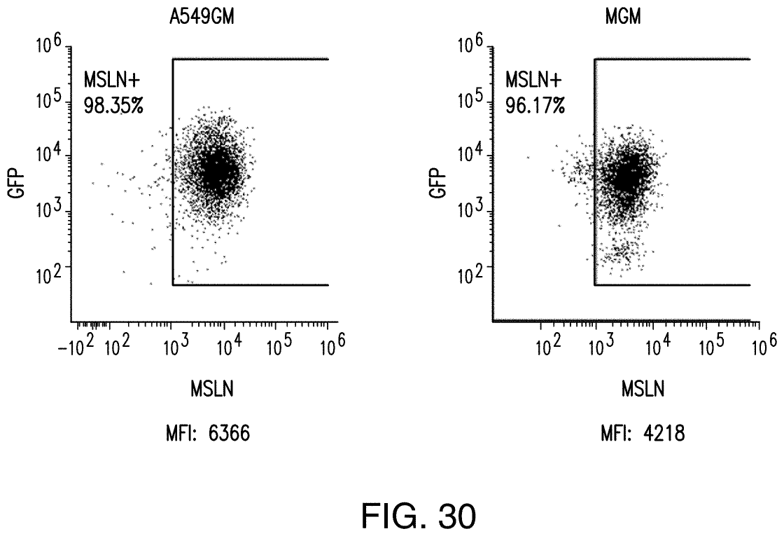

The specifics of soluble PD-1 (sPD-1) and PD-L1 (sPD-L1) used in the experiment were shown. sPD1-Fc and sPD-L1-Fc were used at the concentration of 10 ng/ml and 4 ng/ml, respectively (FIG. 27). The timeline of experiment was presented. M28z or M28z-PD1DNR were pre-treated with sPD-1/sPD-L1/TGFβ at 72 hours prior to transduction analysis and were plated in 48-well tissue culture plate. A5949 or MGM cells were plated to the well containing M28z or M28z-PD1DNR at 24-30 hours before flow analysis for transduction (FIG. 28). T-cells were transduced with M28z and M28z-PD1DNR at day 9 (day of effector CTL (eCTL); cells for flow cytometry were incubated separately at 2×10⁵/200 μl in 96 well plate 3 days before). About 55% transduction efficiency of cells was measured for both M28z and M28z-PD1DNR constructs (FIG. 29).

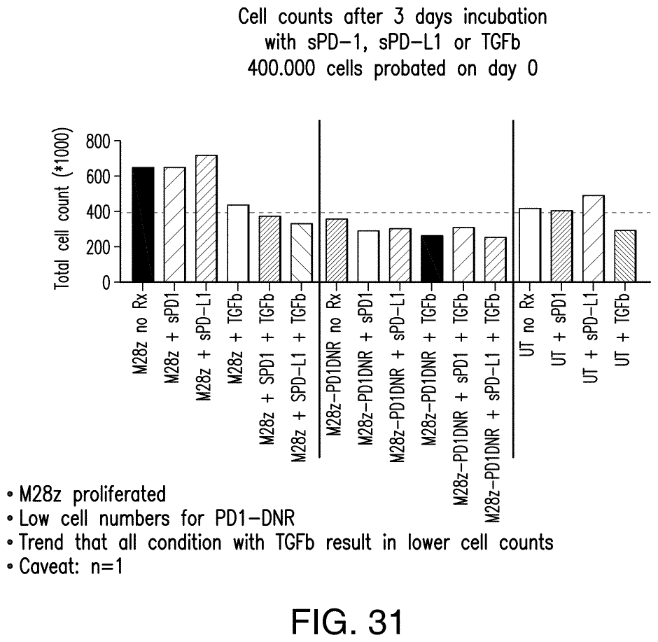

MSLN expression in A549GM (non-small cell lung cancer cells) and MGM (mesothelioma cells) cell lines was measured (FIG. 30). Cells were counted after 3 day incubation with soluble factors sPD-1 and sPD-L1 (no antigen) before plating in eCTL and 4.0×10⁵ cells per well were plated (FIG. 31).

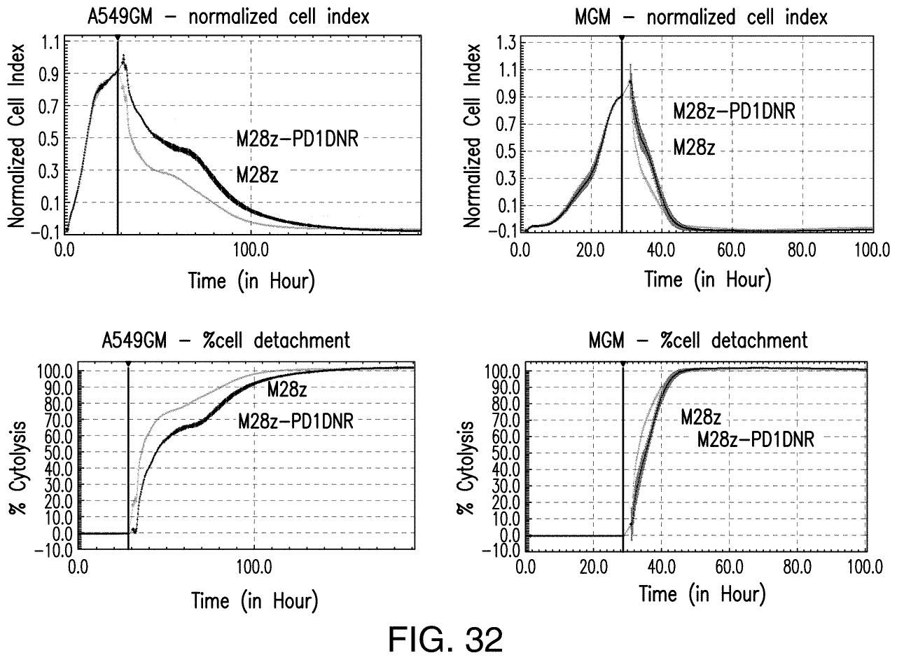

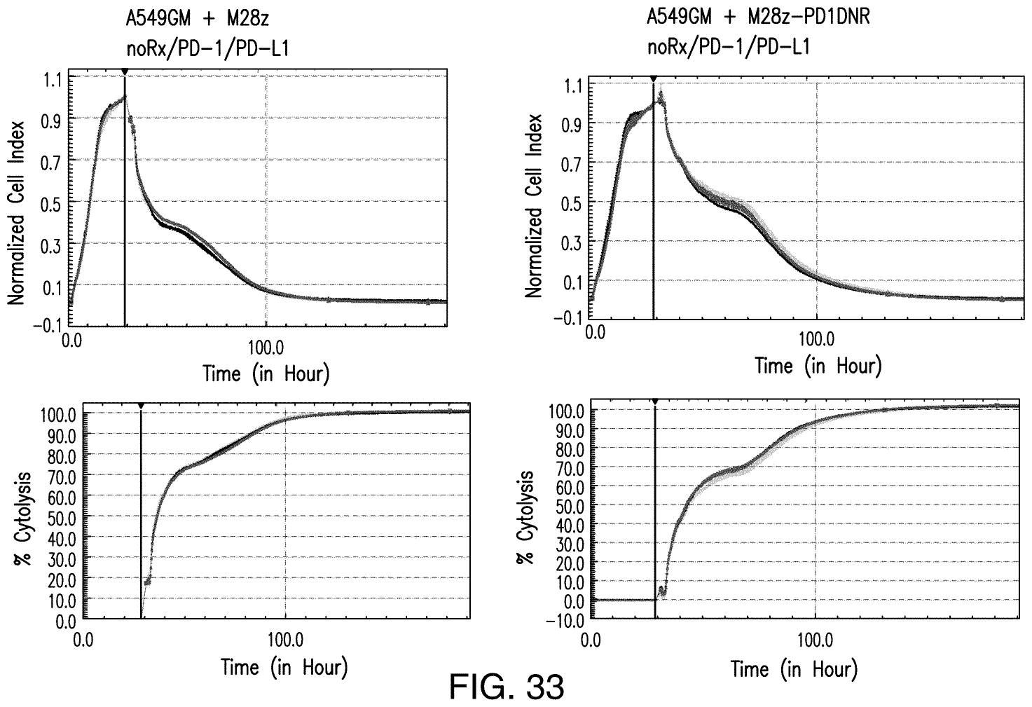

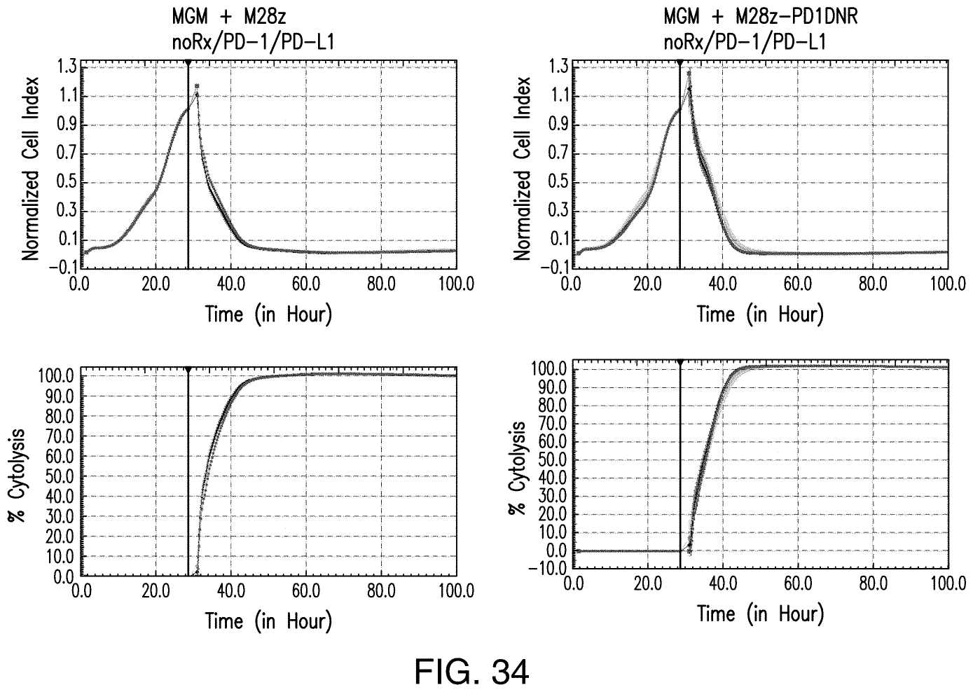

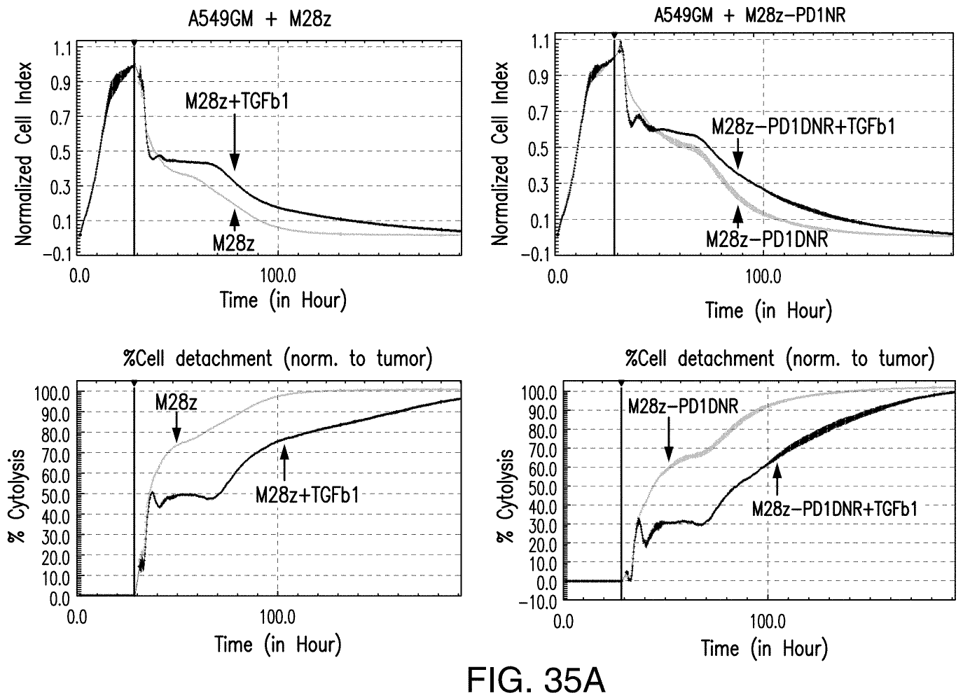

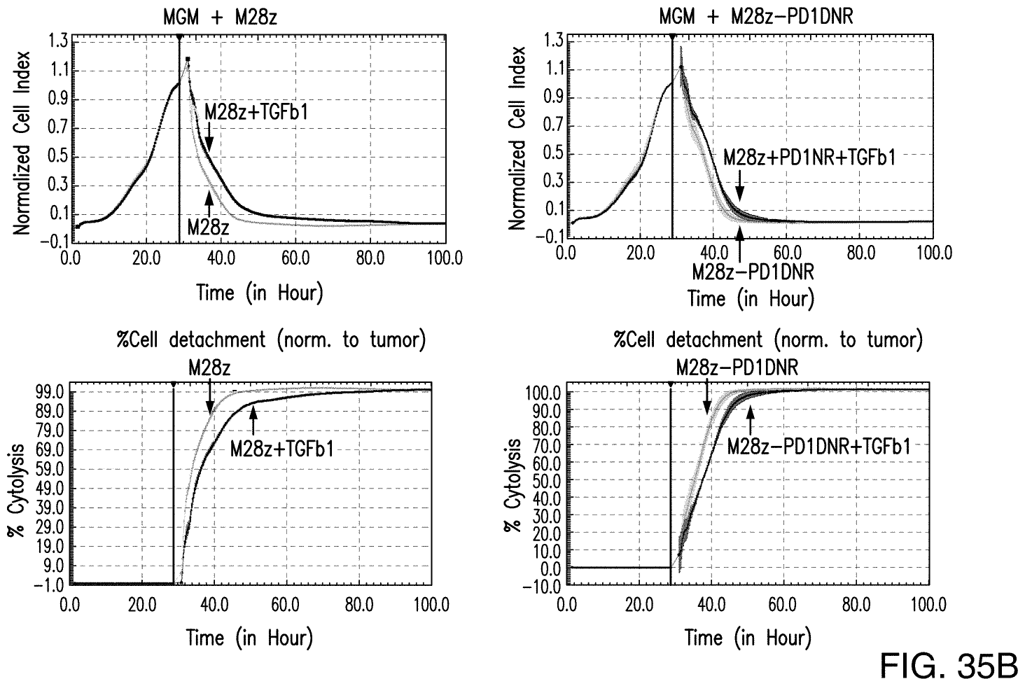

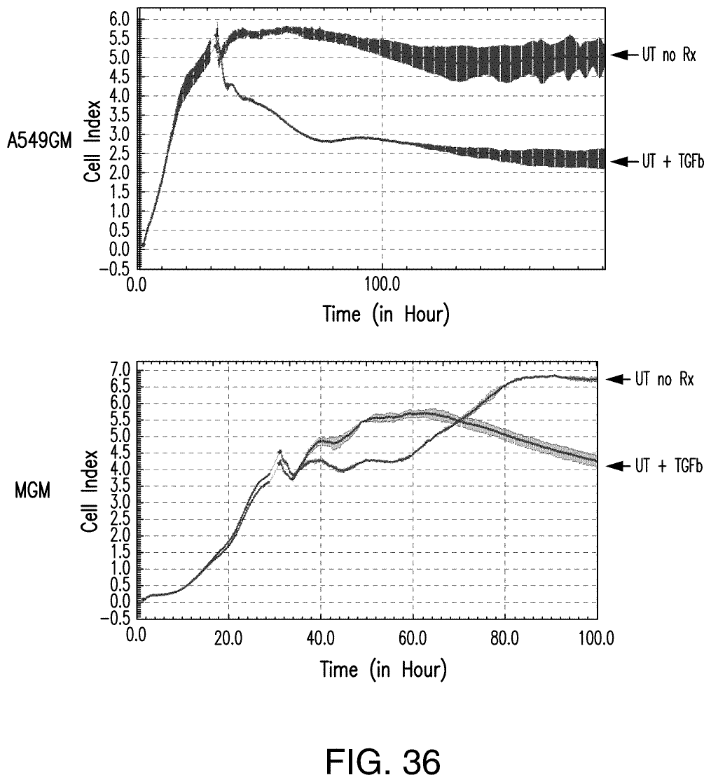

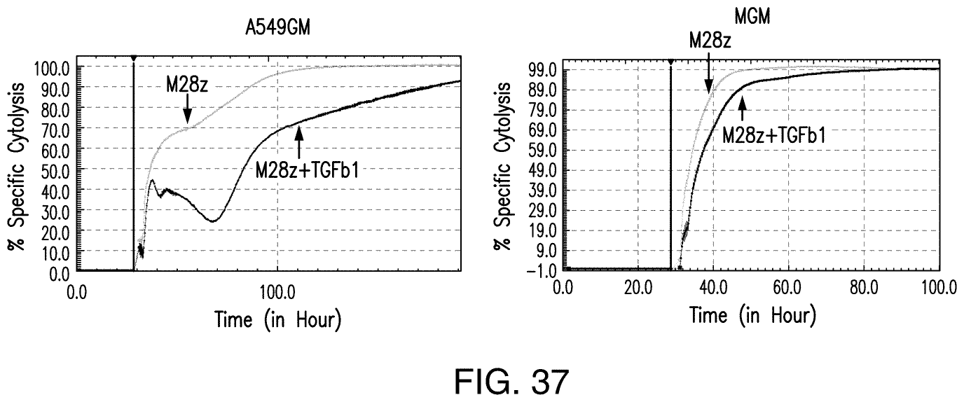

M28z killed A549GM and MGM faster than M28z-PD1DNR at E:T ratio 3:1 without addition of soluble factors. Killing of tumor cells was assessed by cell detachment (FIG. 32). sPD-1 and sPD-L1 did not affect M28z and M28z-PD1DNR cytotoxicity in A549GM and MGM cells (FIGS. 33-34). M28z and M28z-PD1DNR in A549GM cells were about equally inhibited by TGFβ1 (FIG. 35).

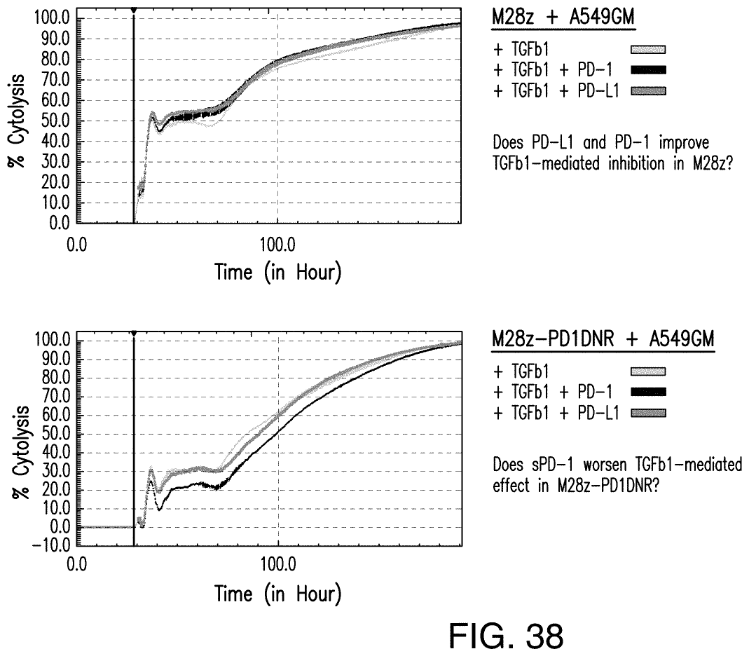

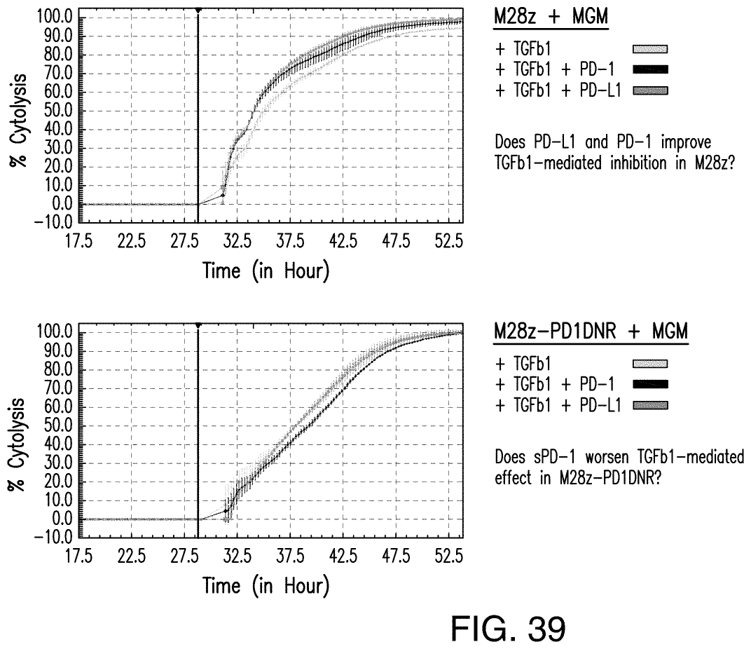

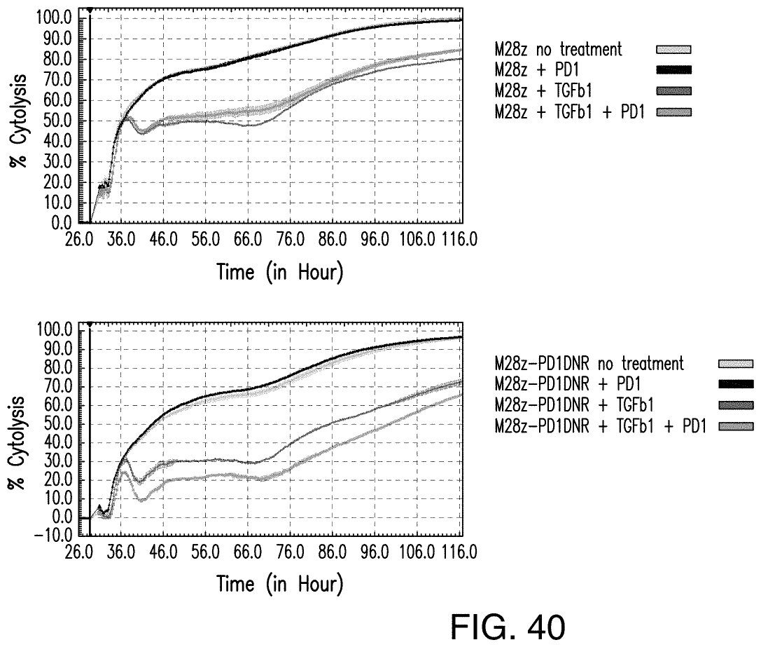

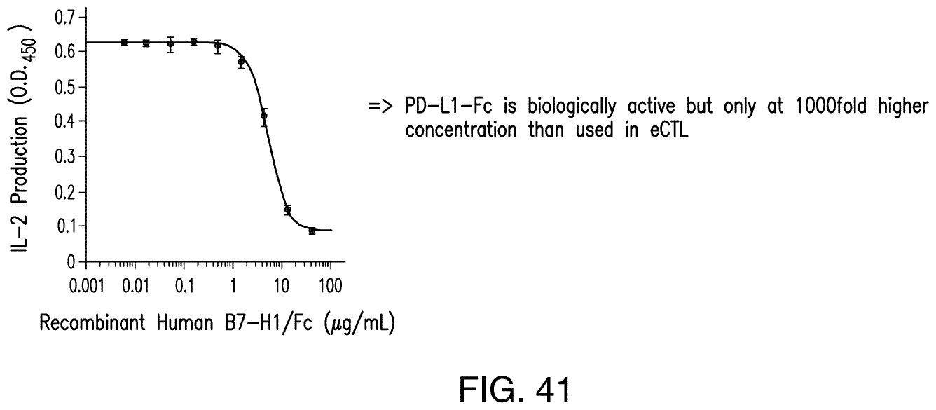

TGFβ1 inhibition in UT T cells in A549GM and MGM cells were exhibited by cell index (FIG. 36). TGFβ1 inhibition was normalized to cell index of tumor cells and UT as mock control (FIG. 37). Effects of sPD-1 and sPD-L1 on TGFβ-mediated inhibition in M28z and M28-PD1DNR were evaluated in A549GM and MGM cells (FIGS. 38-39). M28z vs M28z-PD1DNR towards PD1 and TGFβ1 were compared in A549GM cells (FIG. 40). PD-L1-Fc was biologically active but only at 1000 fold higher concentration than used in eCTL (FIG. 41).

Constructs used in the present example included M28z (LNGFR), M28z-PD1DNR (myc), and M28z-TGFβRIIDNR (planned, cells died after electroporation).

M28z and M28z-PD1DNR were incubated for 3 days in 48W plate (4e5/400 ul) with either of the following treatments (no antigen stimulation):
sPD-1: 10 ng/mL
sPD-L1: 4 ng/mL
TGFβ1: 2 ng/mL
sPD-1+TGFβ1
sPD-L1+TGFβ1

After pre-treatment cells were transferred to 10.000 A549GM or MGM in ePlate at CAR E:T 3:1.

The rationale for testing sPD-L1 in combination with TGFβ is that TGFβ leads to upregulation of surface-bound PD1, possibly increasing the inhibitory effect caused by sPD-L1.

The experiments show that PD1-Fc and PD-L1-Fc might not be representative of the biologically occurring form of these soluble factors in effusion/serum. PD1-Fc only tested in ELISA by manufacturer (biological functionality unknown) functionality of PD-L1-Fc measured by its ability to inhibit anti-CD3 antibody induced IL-2 secretion in human T lymphocytes. The ED50 for this effect is 2-10 μg/mL. Inhibition seen in CTL with effusions high in sPD-1/sPD-L1 might be caused by a synergistic effect. eCTL allows to study TGFβ-mediated inhibition of cytotoxicity. MGM are more sensitive towards CAR and have lower cell index: increase cell number from 10.000 to 20.000 cells/well and/or decrease E:T ratio.

### Example 4: Cell-Free MPE as an Immunosuppressive System to Study CAR T-Cell Efficacy

MPEs can be used to investigate their differential influence on T-cell cytotoxicity depending upon the composition of the MPE and further can be confirmed by addition of antagonist to the cytokine, in this example by addition of anti-TGFbeta antibody to confirm that T-cell cytotoxicity can be rescued.

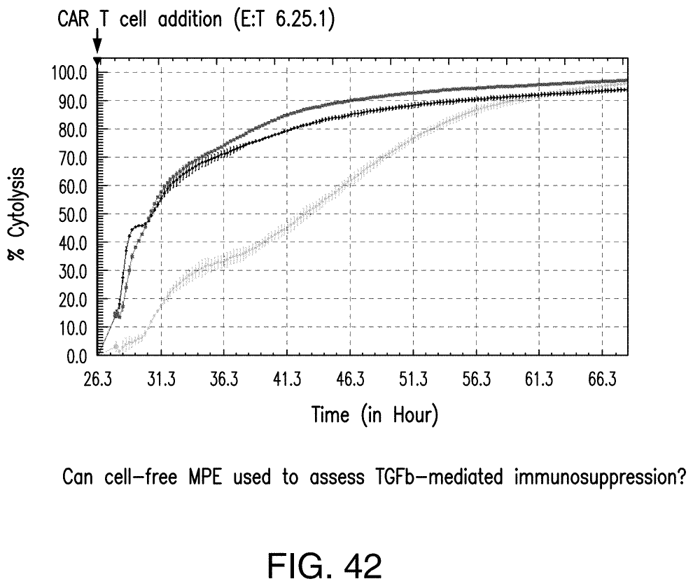

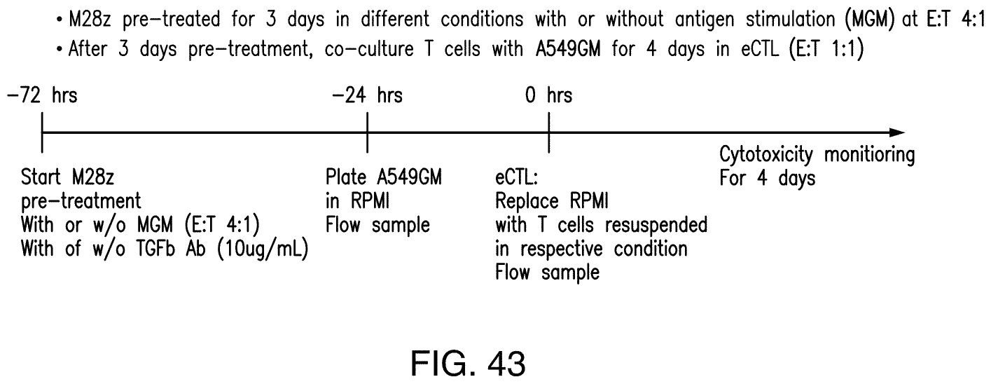

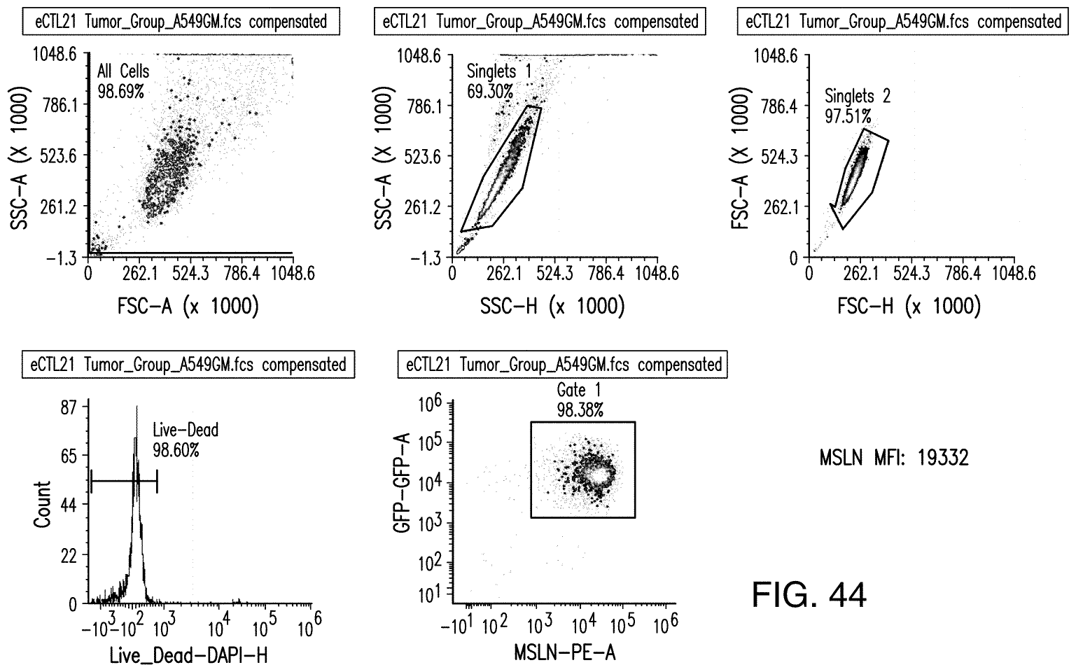

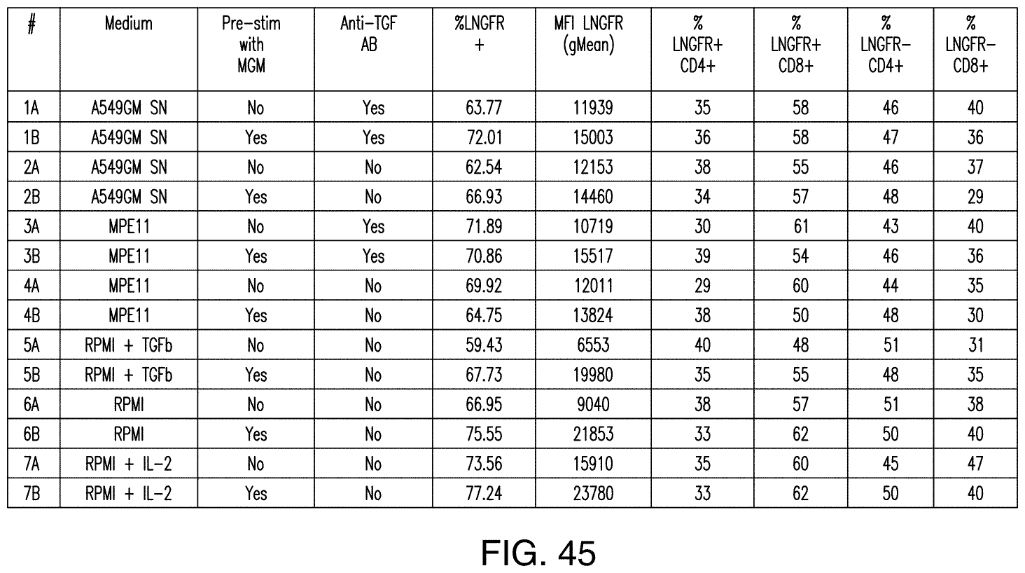

Cell-free MPE was used as an immunosuppressive system to study CAR T-cell efficacy (FIG. 42). Treatment conditions and timeline for coculture of T cells with A549GM cells was depicted in FIG. 43. eCTL21 in A549GM cells were analyzed using flow cytometry. T cells were analyzed using flow cytometry. Columns represent antigen activation status of CAR T cells, whether TGFβ antibody was added or not, percentages of total T cells with CAR expression, and percentages of CD4 and CD8 CAR T cells with CAR expression (FIGS. 44-45).

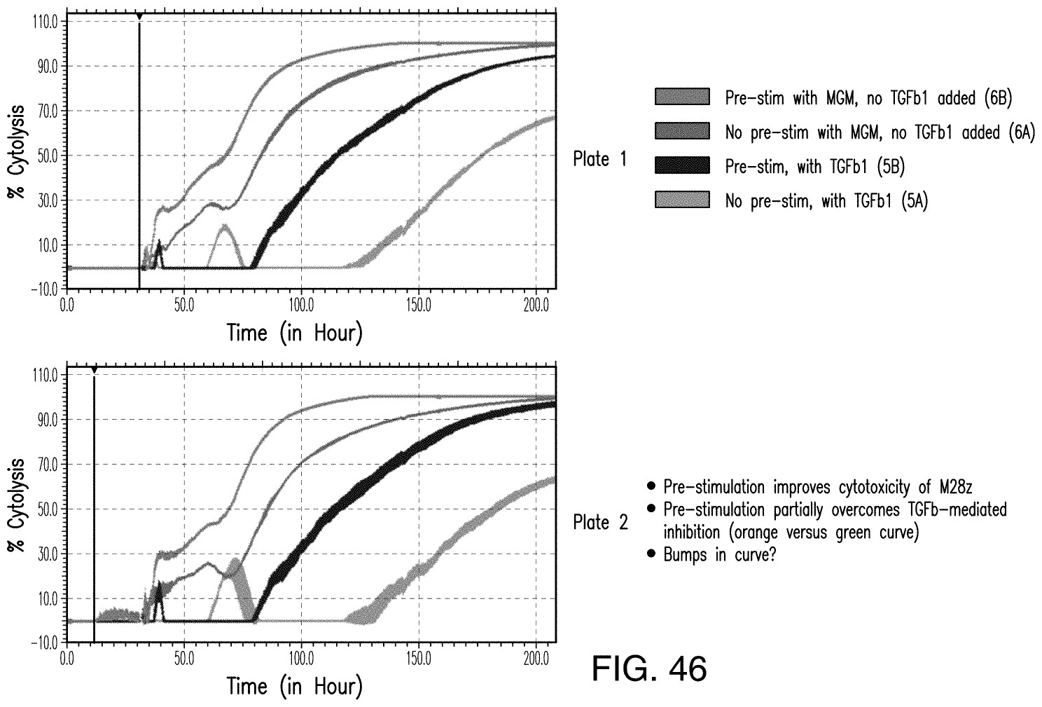

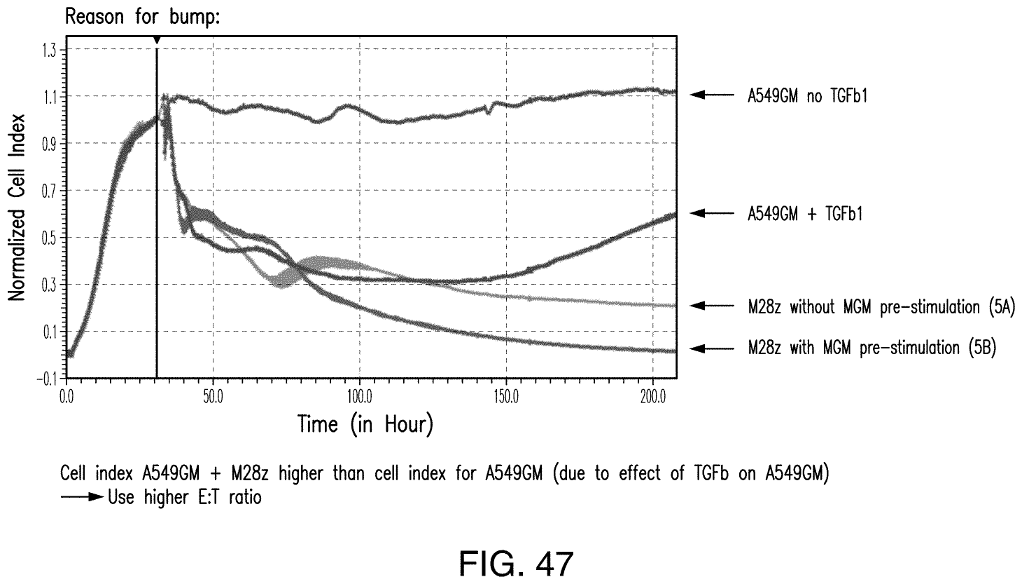

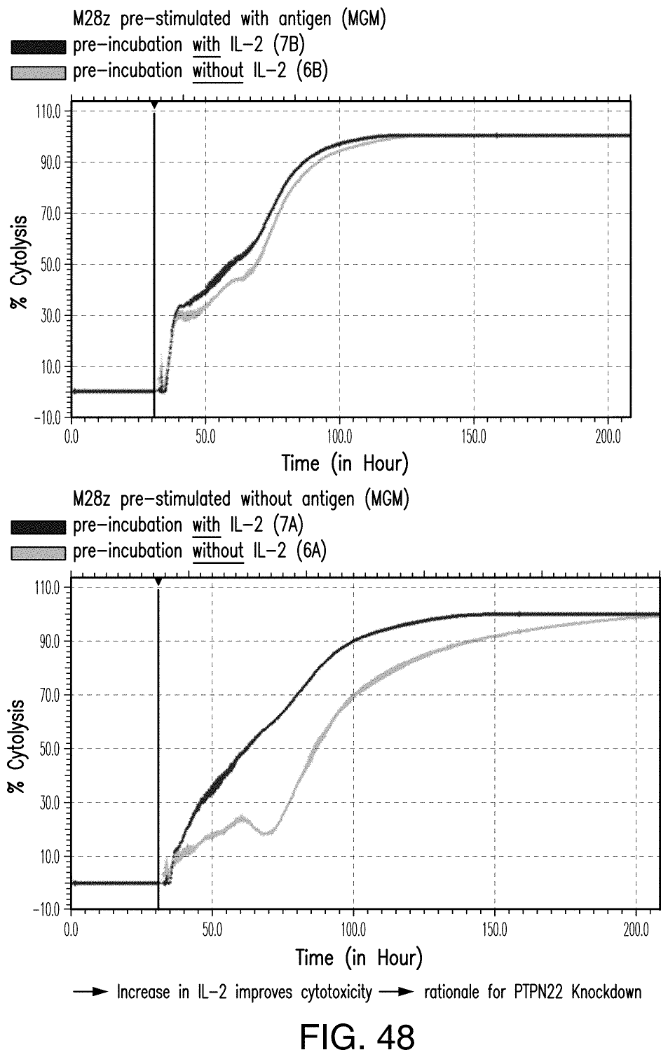

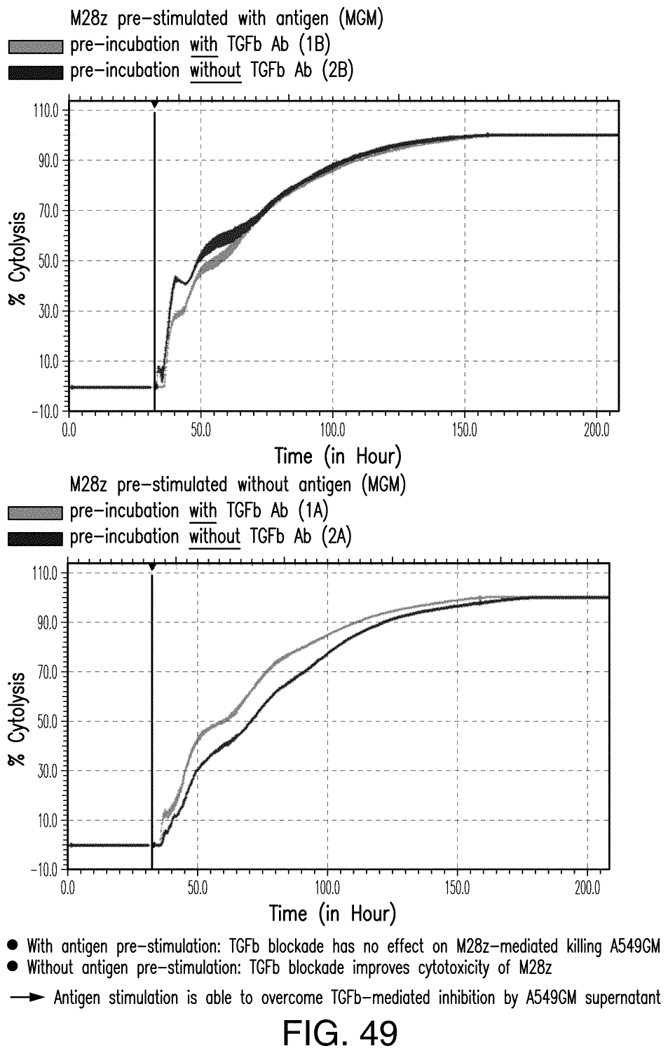

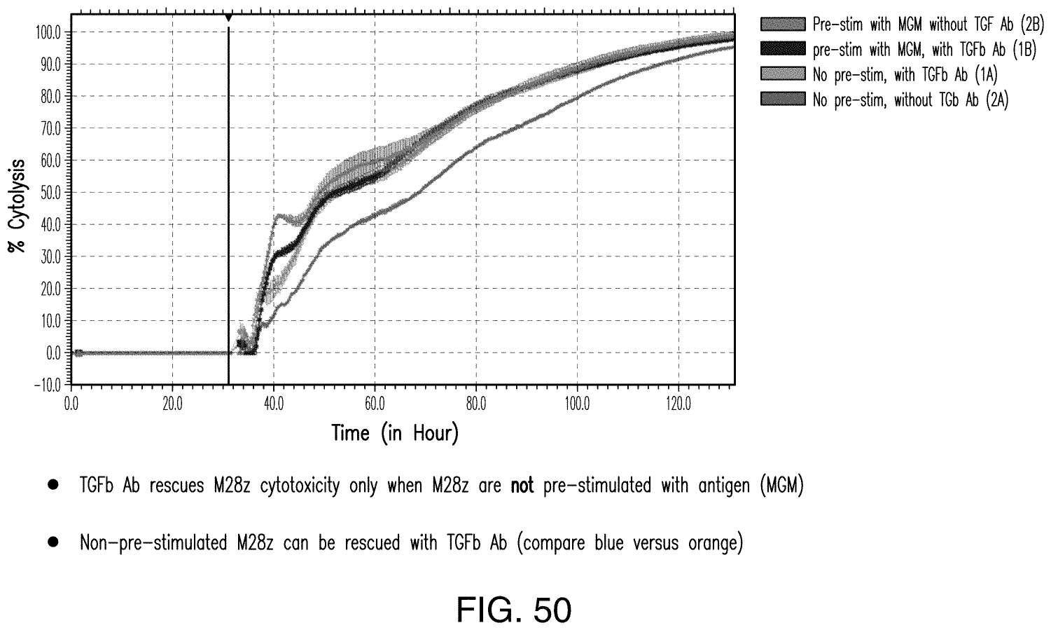

Effects of pre-stimulation with recombinant TGFβ1 on cytotoxicity of M28z in RPMI+10% FCS (no IL-2) were evaluated. M28z pre-stimulated with antigen MGM improved cytotoxicity (FIG. 46). Cell index of M28z with recombinant TGFβ (no IL-2) for A549GM cells was measured (FIG. 47). Addition of IL-2 improved cytotoxicity of M28z, without TGFβ1 addition (FIG. 48). M28z stimulated with antigen MGM was able to overcome TGFβ-mediated inhibition by A549GM supernatant (FIG. 49). TGFβ antibody rescued M28z cytotoxicity only when M28z were not pre-incubated with antigen MGM. M28z without pre-stimulation with MGM could be rescued with TGFβ antibody (FIG. 50).

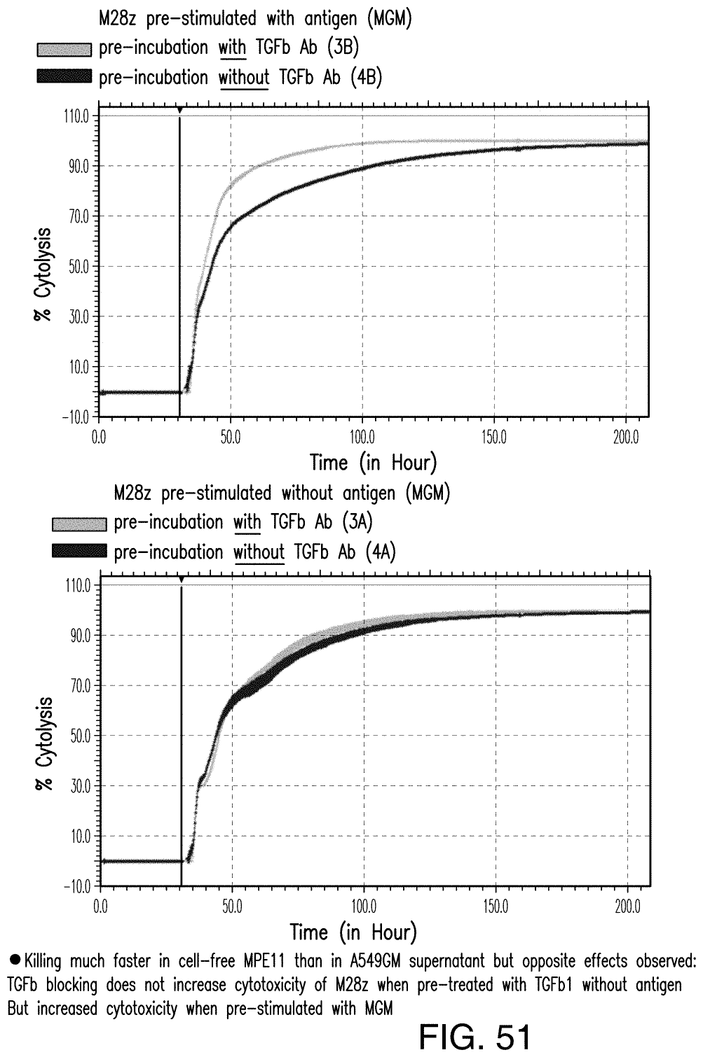

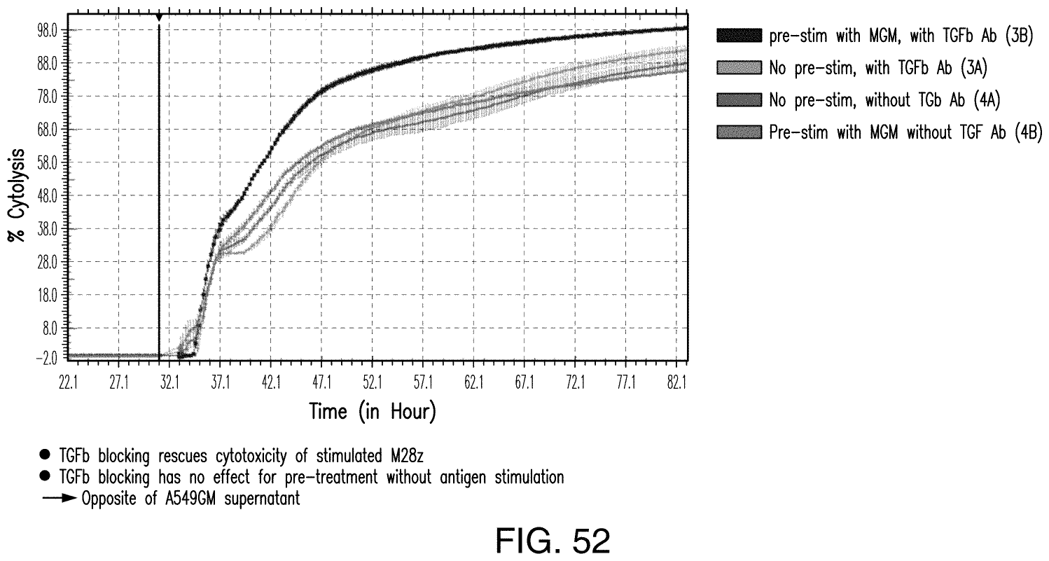

M28z were pre-stimulated with MGM and pre-incubated with or without TGFβ antibody. Killing was much faster in cell-free MPE11 than in A549GM supernatant. TGFβ blocking, however, did not increase cytotoxicity of M28z when pre-stimulated with TGFβ1 without antigen but increased cytotoxicity when pre-stimulated with MGM. TGFβ blocking rescued cytotoxicity of stimulated M28z. (FIGS. 51-52).

Treatment conditions are as follows:
1) A549GM SN with TGFβ antibody (10 ug/mL)
2) A549GM SN w/o TGFβ antibody
3) Cell-free MPE11 with TGFβ antibody (10 ug/mL)
4) Cell-free MPE11 without TGFβ antibody
5) RPMI+10% FCS+TGFβ1 (2 ng/mL) (no IL-2)
6) RPMI+10% FCS (no IL-2)
7) RPMI+10% FCS+IL-2 (40 U/mL)

All conditions were with or without MGM stimulation (E:T: 4:1) for 3 days. Conditions with TGFβ1 antibody were incubated for 2 hrs at 37° C. before use. All pre-treatment conditions were maintained during the experiment (including eCTL). TGFβ1 concentrations were: about 2 ng/ml in A549GM; 10 ng/ml in MPEP11; and 2 ng/ml in recombinant TGFβ1.

The present example shows that MPE11 can be used as an immunosuppressive system to assess efficacy of M28z constructs engineered to overcome TGFβ-mediated inhibition Prior antigen-stimulation before eCTL is required.

### Example 5: Investigation of PE41 Cells for their Potential Use as an In Vivo Tumor-PE Coculture Model

MPE influence on CAR T cells against non-small cell lung cancer cells was investigated in the presence of multiple target and effector cells.

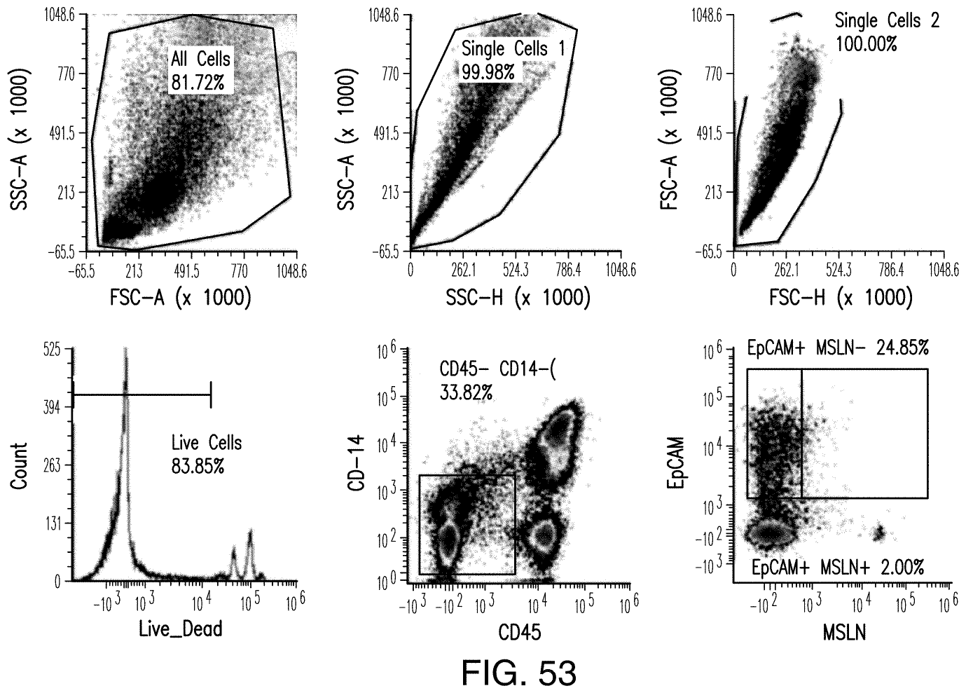

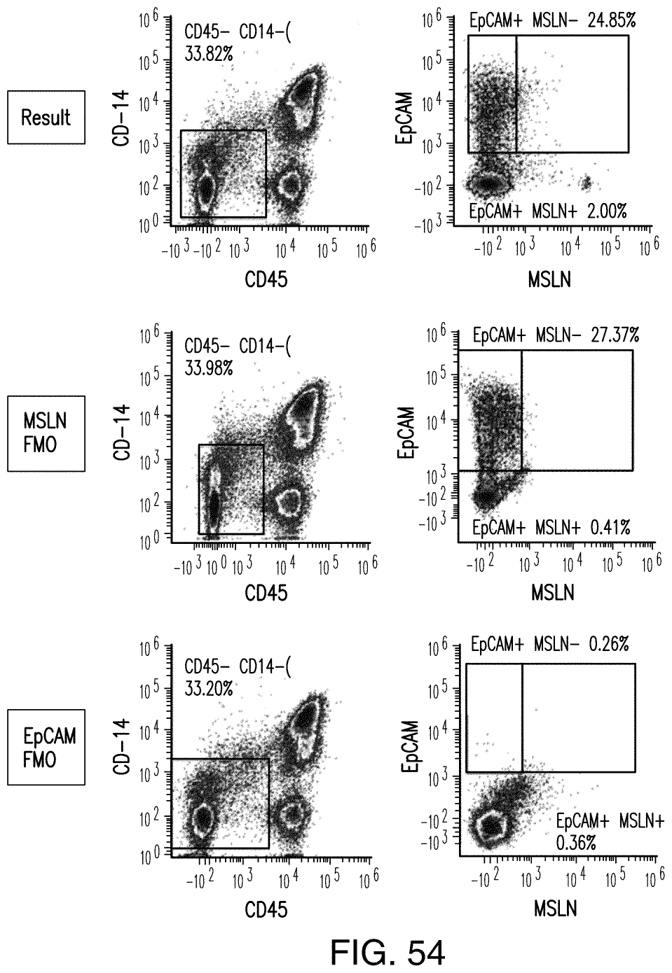

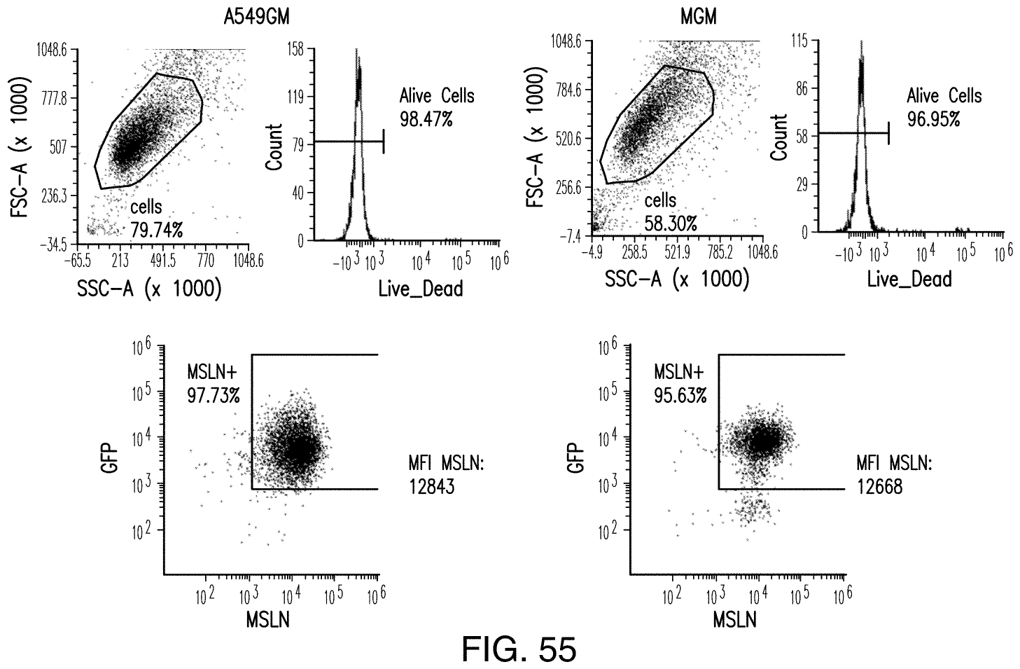

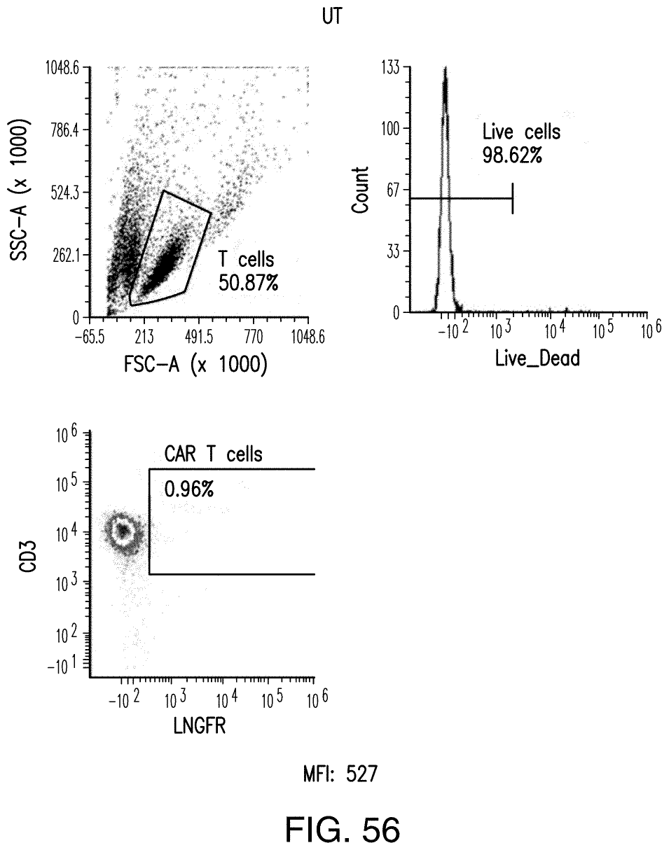

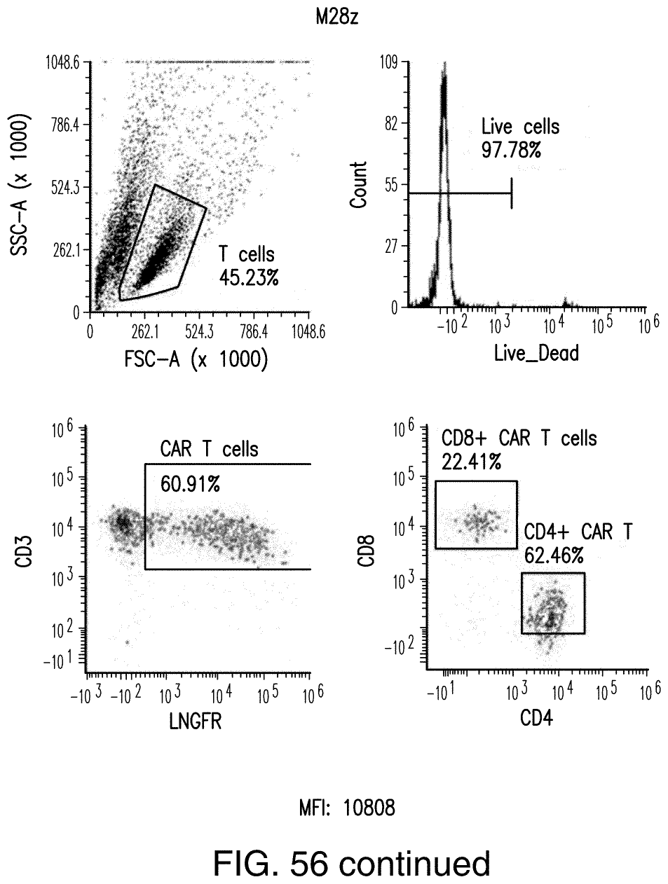

MPE41 tumor panel gating strategies for flow cytometry analysis of CD45−/CD14− and EpCAM+MSLN-cells was represented in FIG. 53. The results with The effect of FMO on the cell population for CD45−/CD14− and EpCAM+MSLN-cells were shown in FIG. 54. MSLN/GFP expression in A549GM and MGM tumor cells was analyzed using flow cytometry before plating (FIG. 55). M28z transduction efficacy was evaluated for CD8+ CAR T cells (FIG. 56).

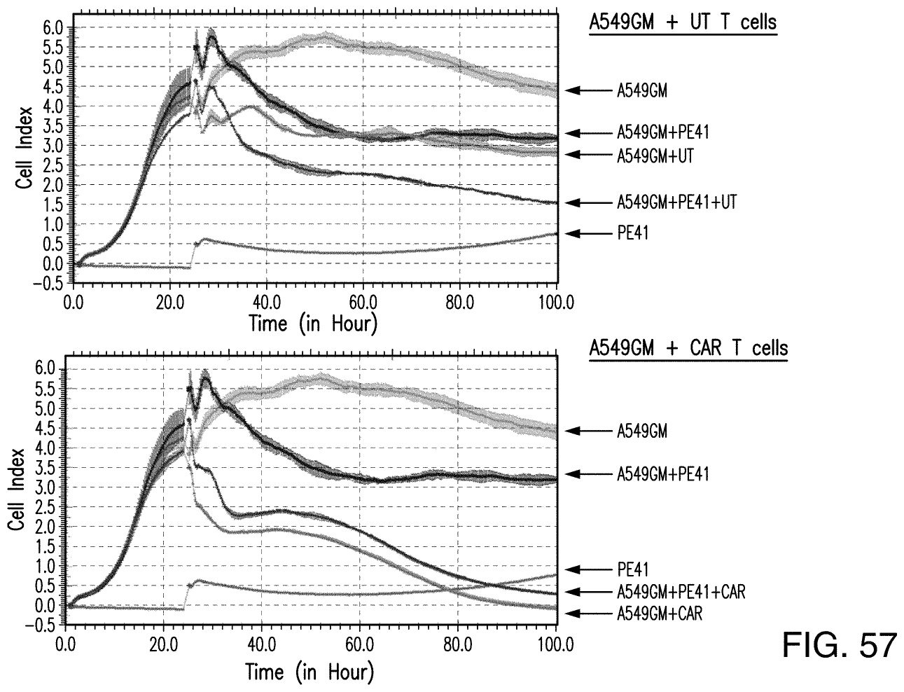

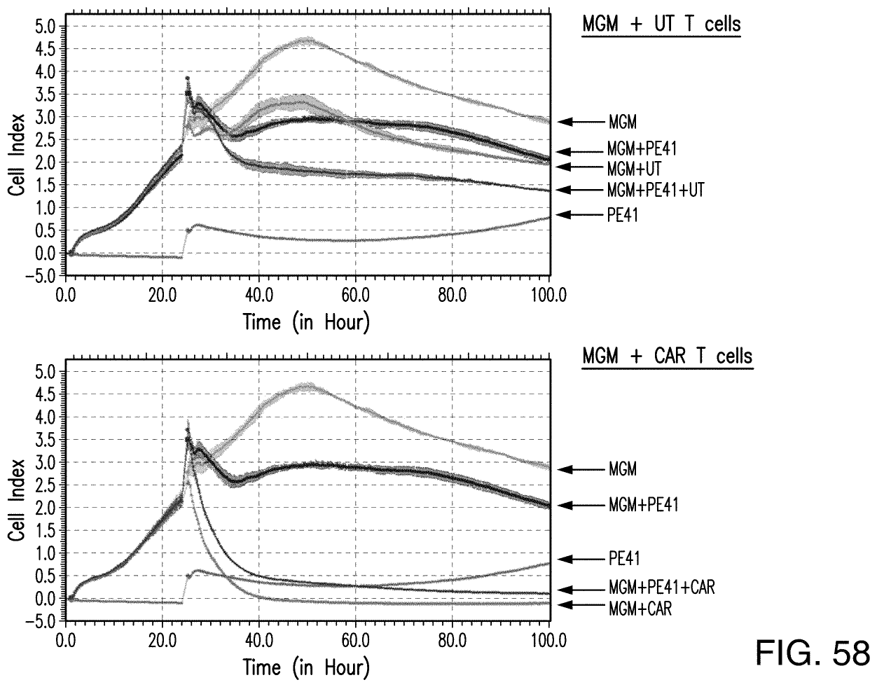

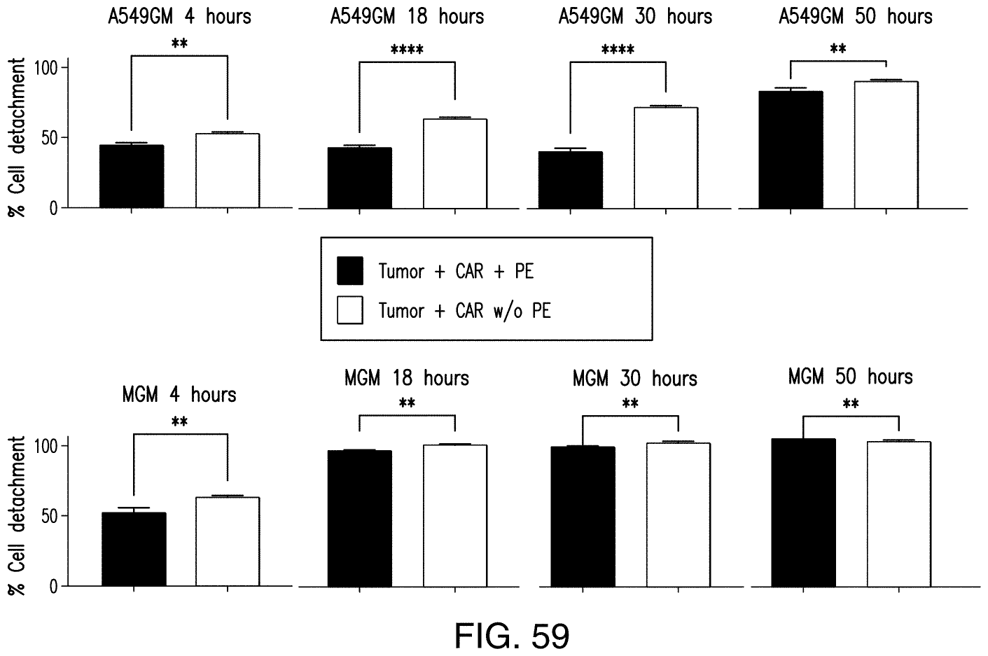

Impact of PE41 on A549GM and T-cell mediated killing were shown in FIG. 57. MGM and T-cell mediated killing was evaluated (FIG. 58). CAR T cell efficacy in presence of PE41 cells (normalized) was compared with A549GM and MGM cells (FIG. 59).

To characterize CAR T cell efficacy in presence of PE41 cells with the eCTL, experiment were conducted under the following conditions:

10000 A549GM or MGM cells plated 24 hours before PE41 and CAR T cell addition

1×10⁶ PE41 cells plated, directly used after thawing

Fixed CAR E:T ratio of 6.25:1

To accommodate for background of PE41 cells:

Cell index (Tumor+PE)—Cell index (PE only)
Cell index (Tumor+CAR+PE)—Cell Index (PE only)
Calculation of % detachment to compare groups with and without PE41 cells:

Formula for condition with PE41 for a specific time point:

$$\frac{\begin{matrix}Normalized cell index ( Tumor + CAR + PE ) - \\ normalized cell index ( Tumor + PE )\end{matrix}}{{0}^{*} - Normalized cell index ( Tumor + PE )}$$

Formula for condition without PE41 for a specific time point:

$$\frac{Cell index ( Tumor + CAR ) - Cell index ( Tumor only )}{{0}^{*} - Cell index ( Tumor only )}$$

$${}^{*} corresponds to 100 \% cell lysis ( cell index = 0 )$$

Assumption is no crowding effect.

PE41 showed immunosuppression when data is normalized. Effects observed are comparable to Cr51 CTL with cell-free effusion. Normalization must be used with caution as the complex interaction between PE41 cells and tumor and CAR is oversimplified.

### Example 6: Formation of Multicellular Tumor Spheroids (MCTS) with Malignant Pleural Effusion (MPE) Immune Cells

As T cell cytotoxicity is influenced by the inhibitory macrophages within the tumor micro environment, M2 macrophages obtained from MPEs can be used to investigate their influence on CAR T-cell cytotoxicity. In this example, macrophages were co cultured in a spheroid model with or without target cancer cells as well as with or without radiation therapy and with or without? addition of macrophage stimulatory factors such as CSF-1. CAR T-cell growth, cytotoxicity on target cells was investigated following incubation with target cells.

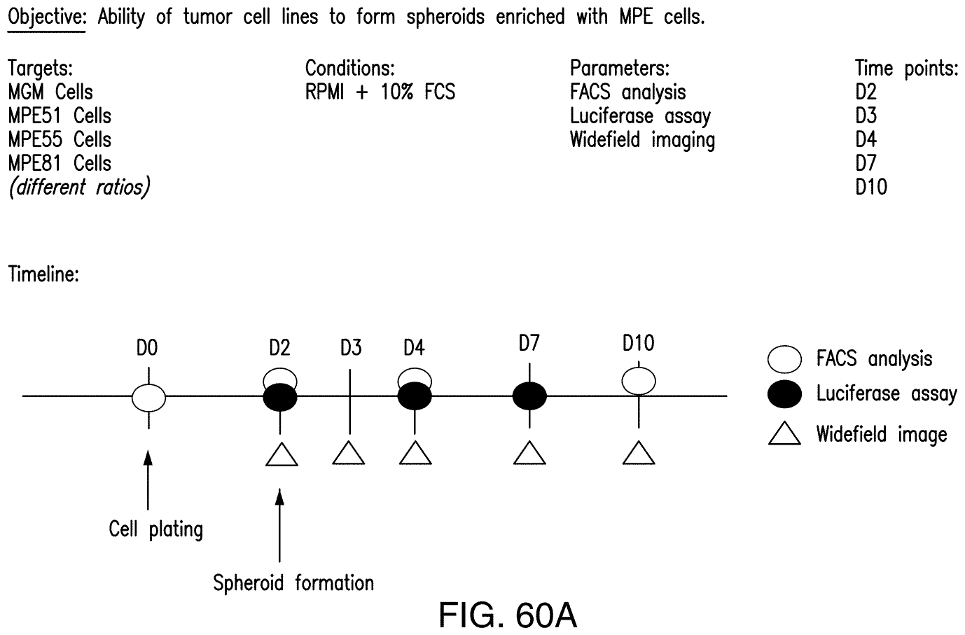

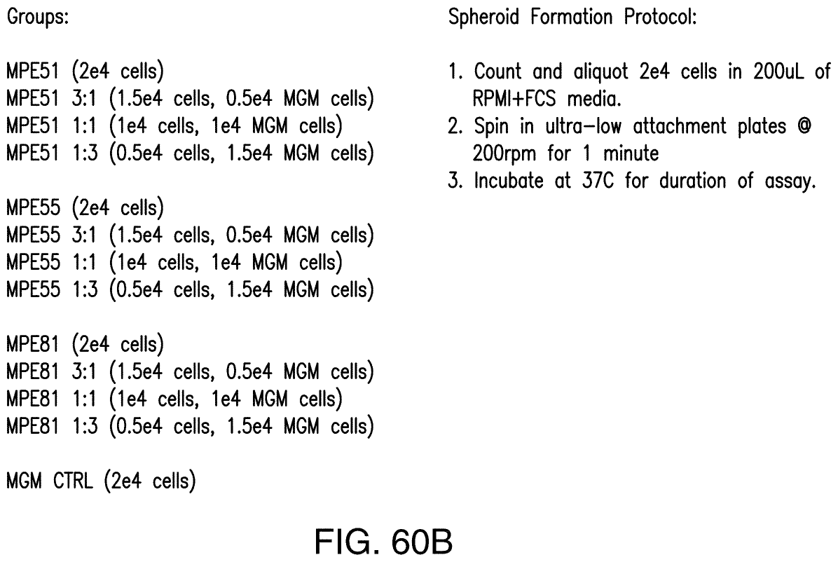

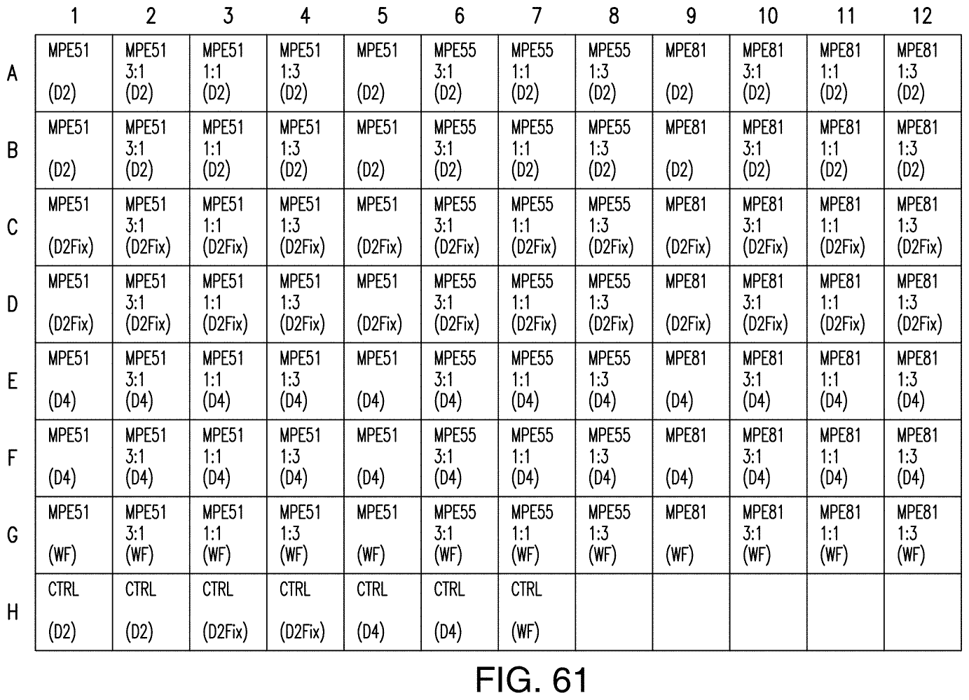

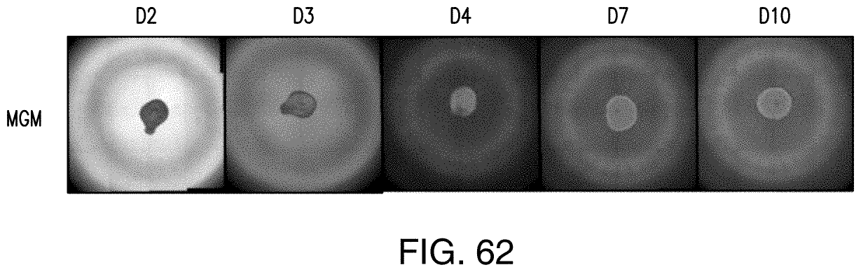

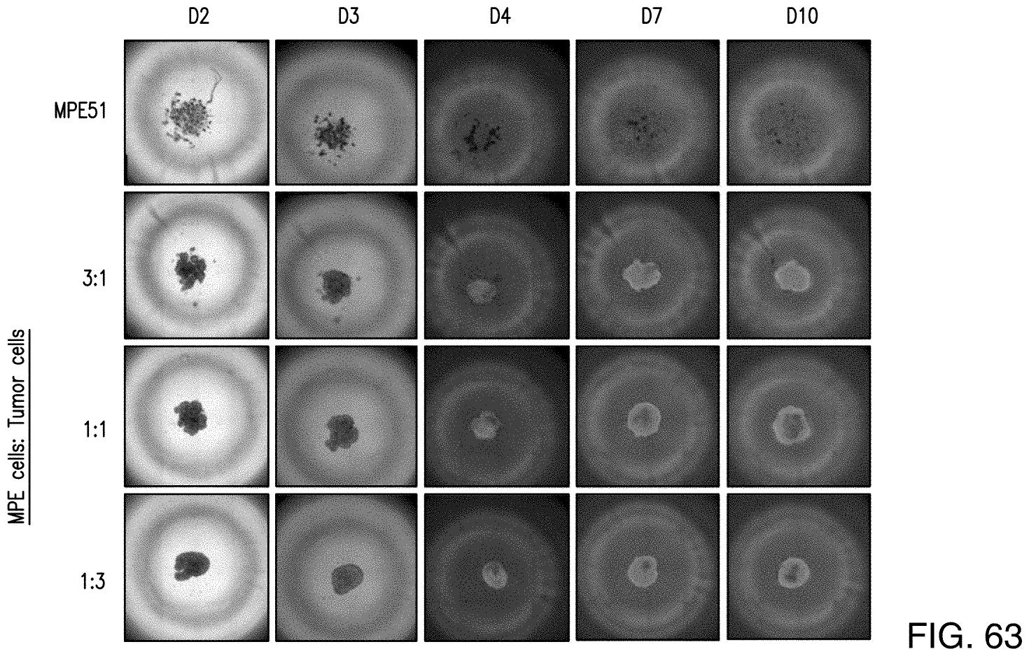

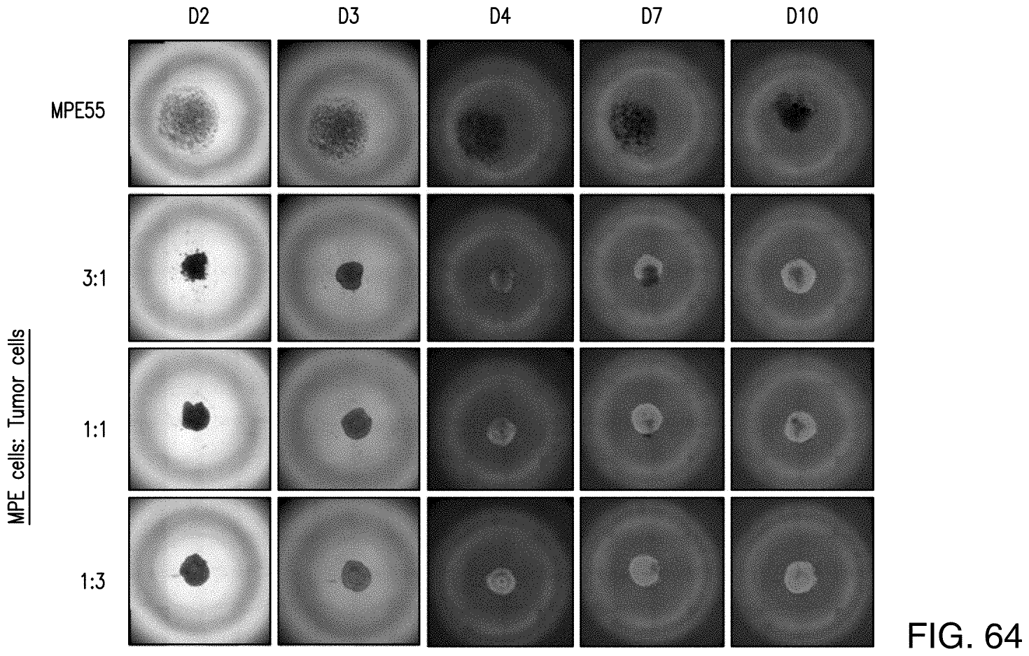

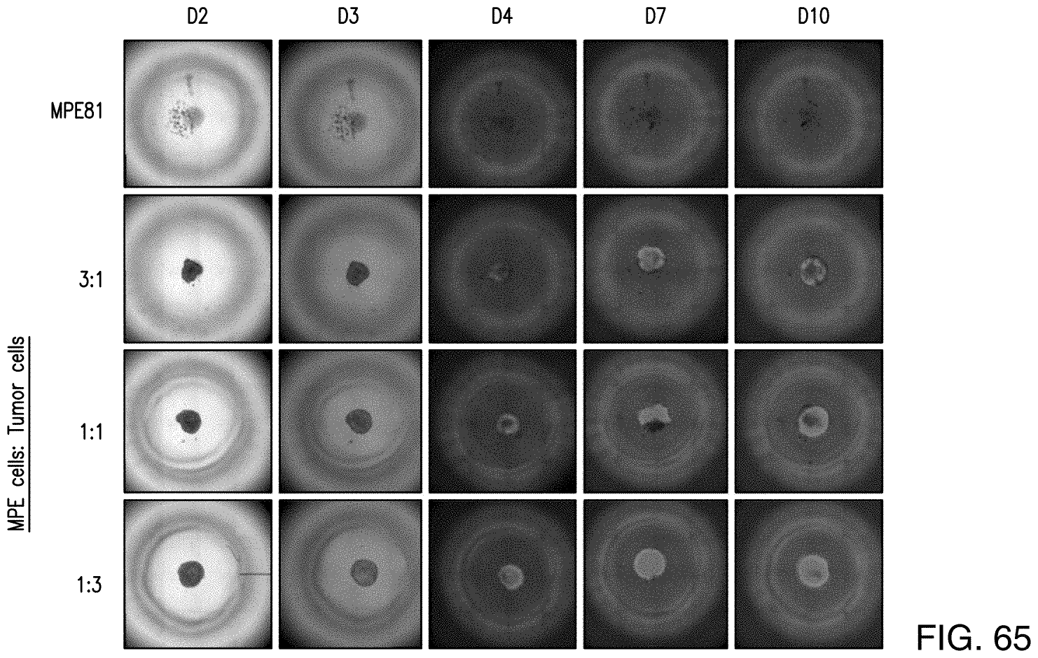

The experimental scheme for detecting ability of tumor cell lines to form spheroids enriched with MPE cells, including target cells, conditions, timeline and analysis methods was depicted in FIG. 60 and groups and spheroid formation protocol were depicted in FIG. 61. ZEN microscope brightfield and GFP merged images over time (FIG. 62). ZEN microscope brightfield and GFP merged images in MGM cells incubated with MPE51, MPE 55, and MPE 81 were shown in FIGS. 63-65 respectively at the various ratio of MPE cells:Tumor cells were photographed over time.

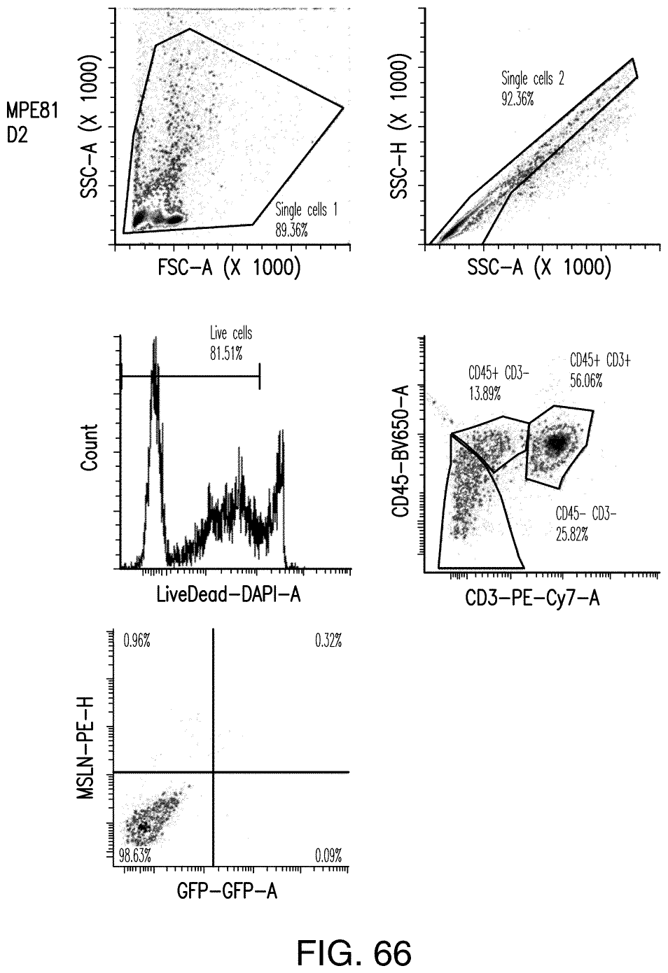

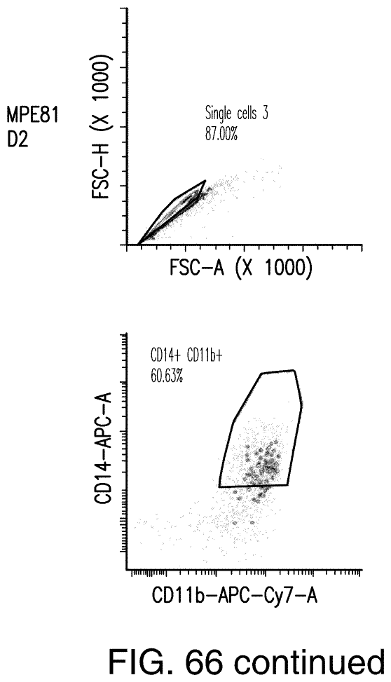

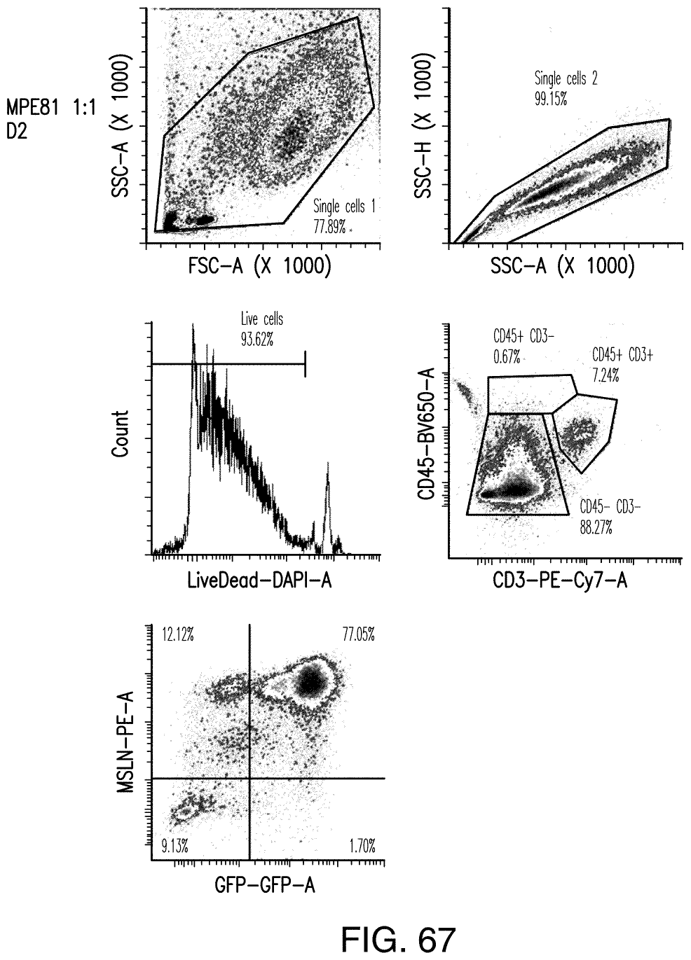

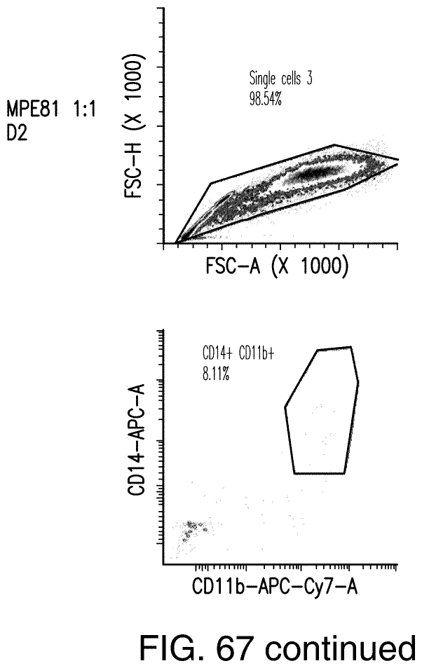

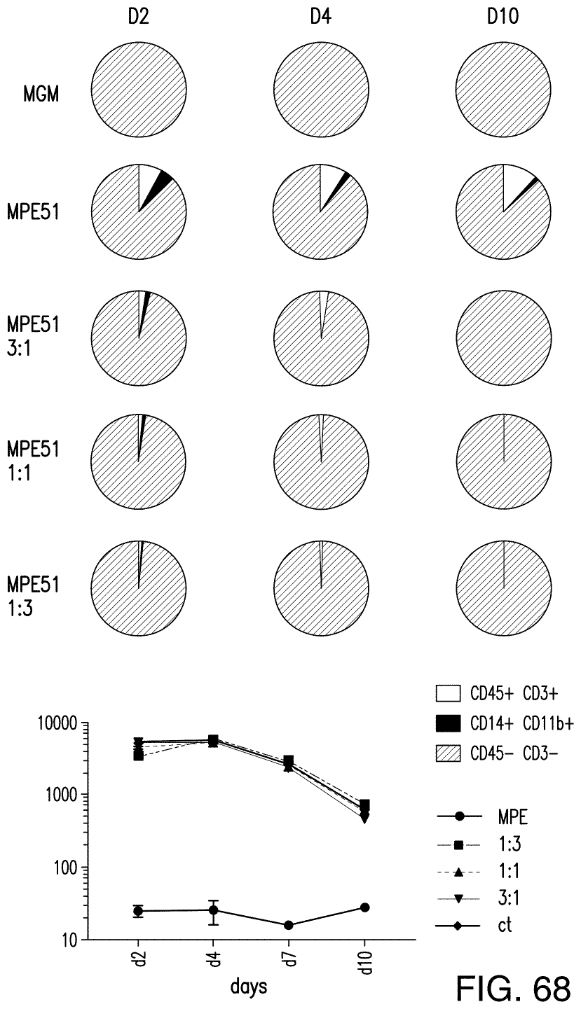

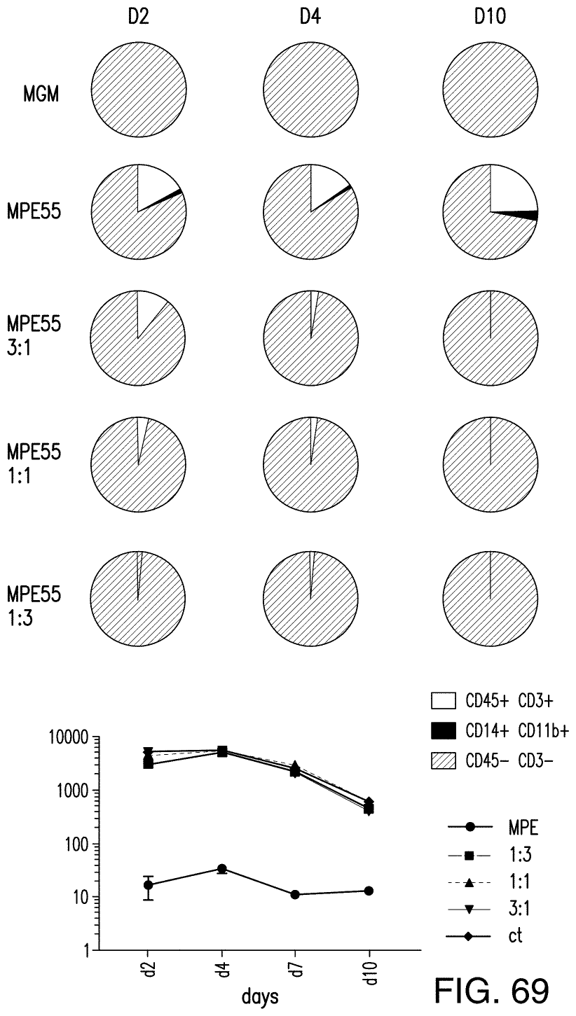

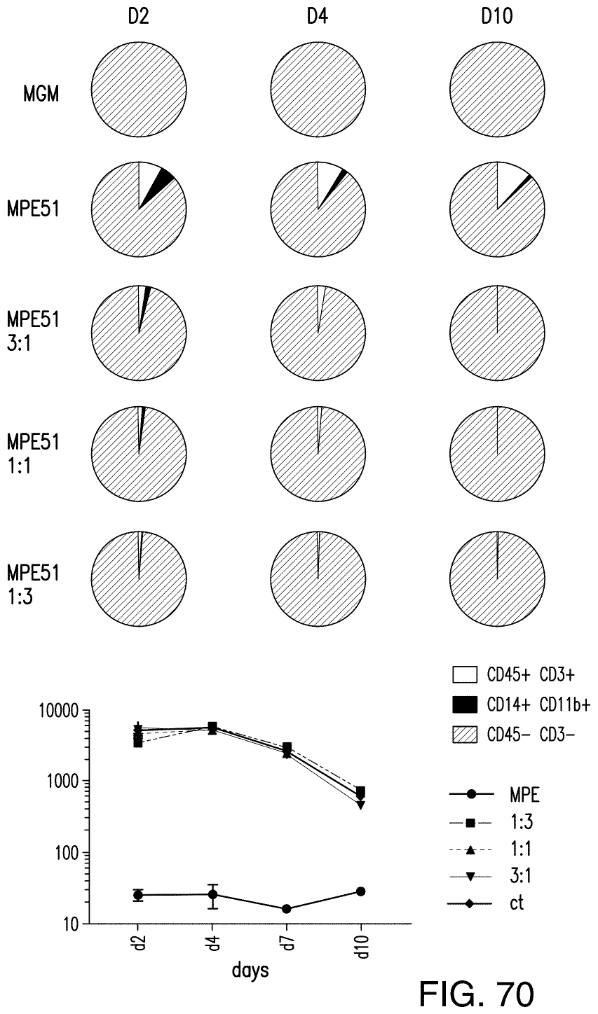

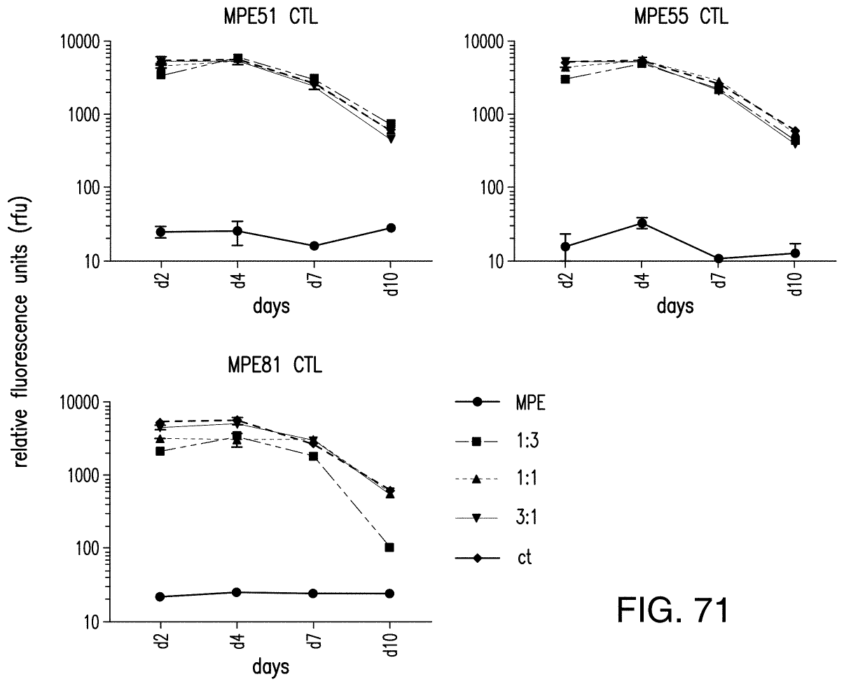

Flow cytometry gating strategies for MPE 81 for CD45+CD3+ and CD14+CD11b+ cells at day 2 were shown in FIGS. 66-67. Flow cytometry analysis of cell populations for MPE 51 cells and MPE 55 cells at the various ratio of MPE cells:T cells were shown (FIGS. 68-70). MGM tumor cell populations were evaluated using luciferase activity assay (FIG. 71).

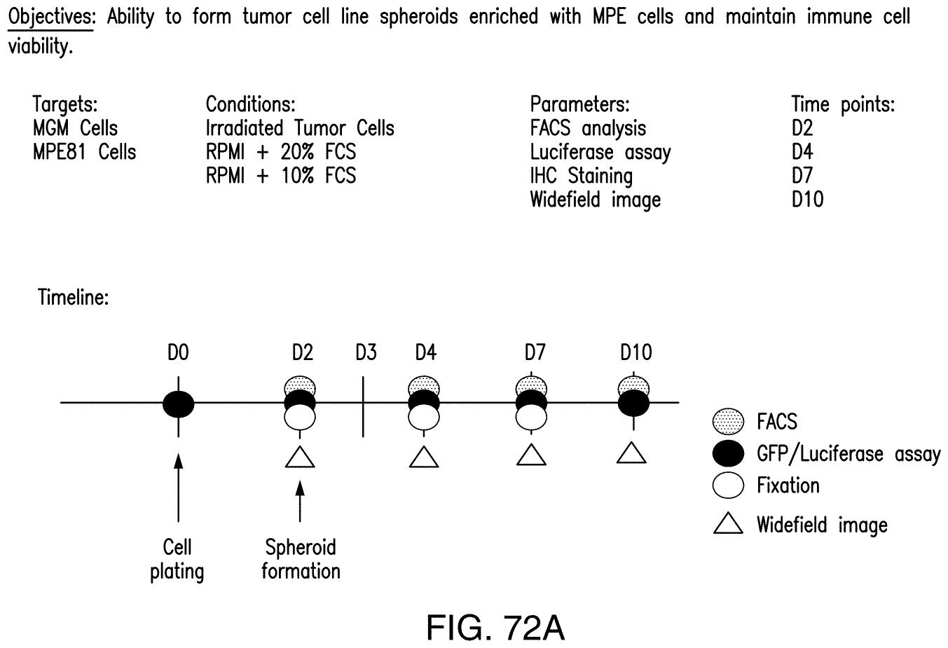

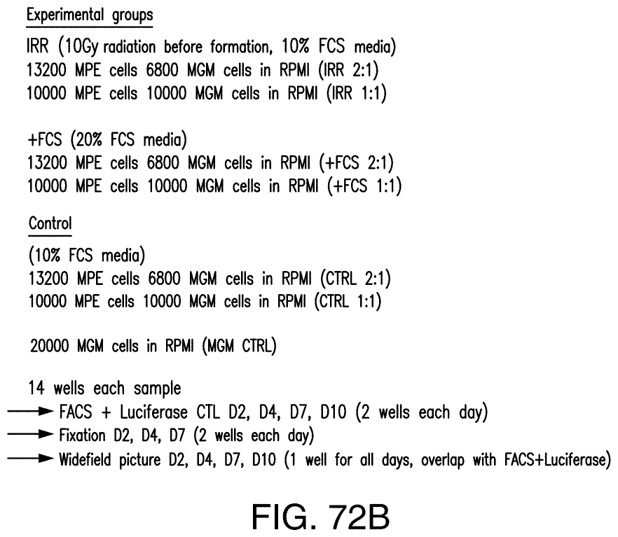

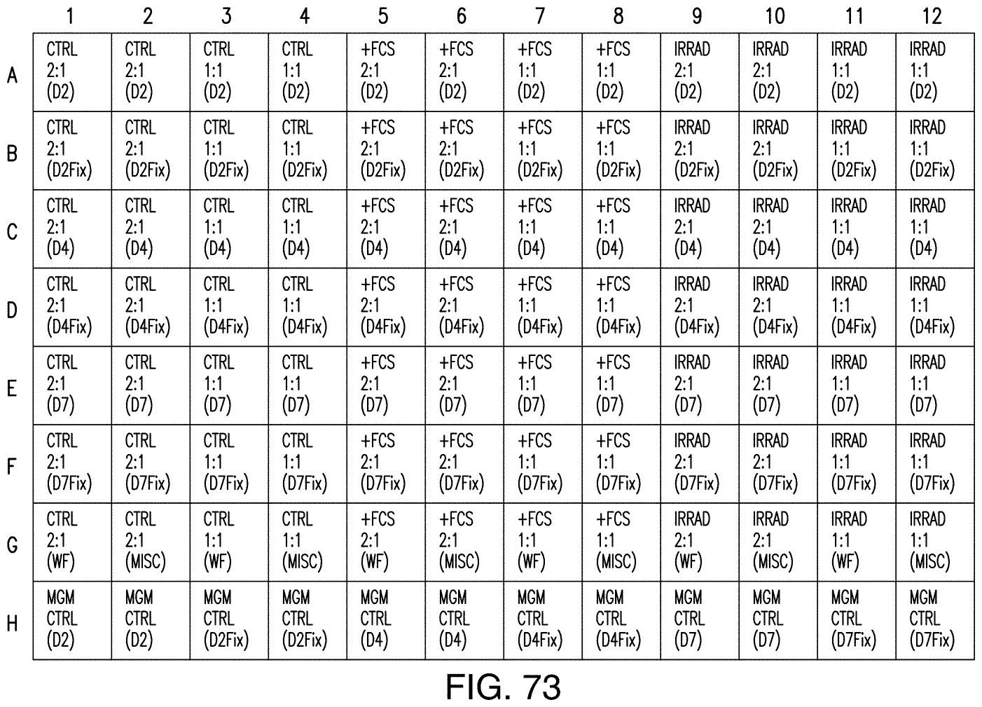

Experimental scheme to identify the ability to form MGM tumor cell line spheroids enriched with MPE cells and maintain immune cell viability was laid out. Target cells were MGM and MPE 81 cells. Cells were incubated with or without different doses of radiation or with or without addition of CSF1 factor at different concentrations. MPE cells and MGM cells were mixed at the ratio of 2:1 or 1:1 in RPMI cell culture medium, and were plated in a 96 well tissue culture plate. The cells were then analyzed using FACS assay and Luciferase assay at day 2, day 4, day 7 and day 10. Cells were fixed at day 2, 4 and 7 and widefield images were pictured at day 2, day 4, day 7 and day 10 (FIGS. 72-73).

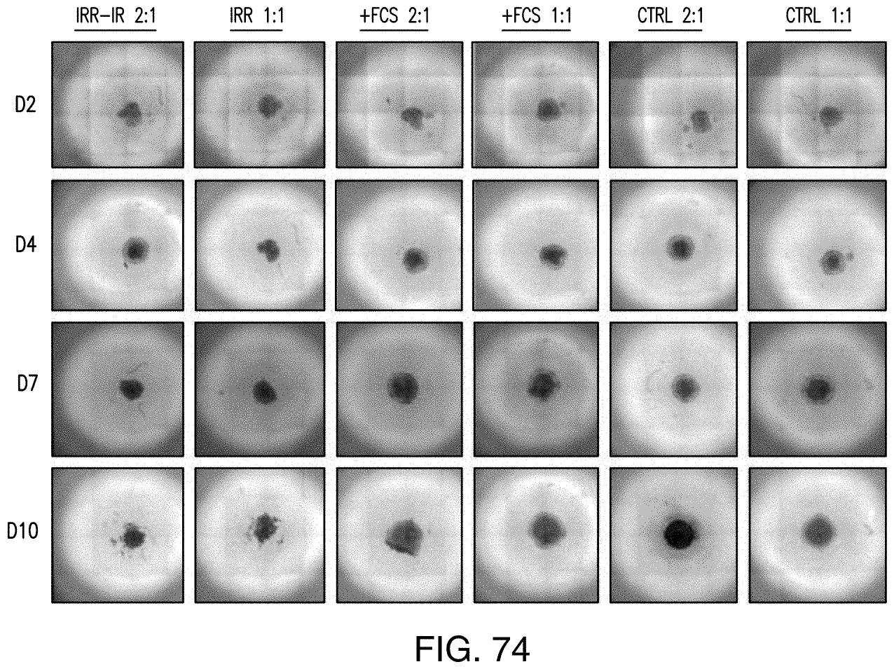

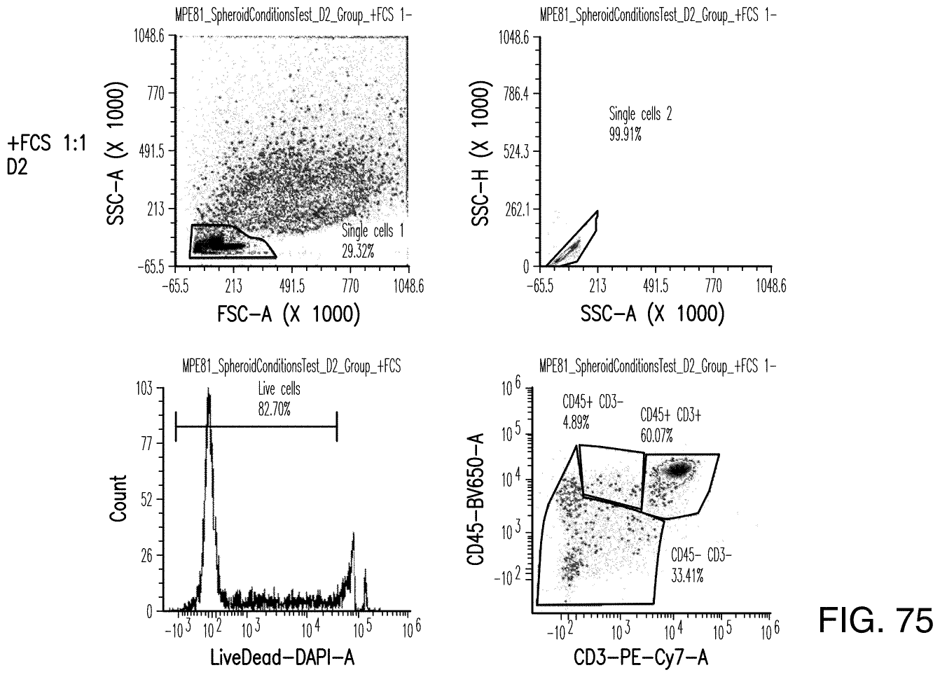

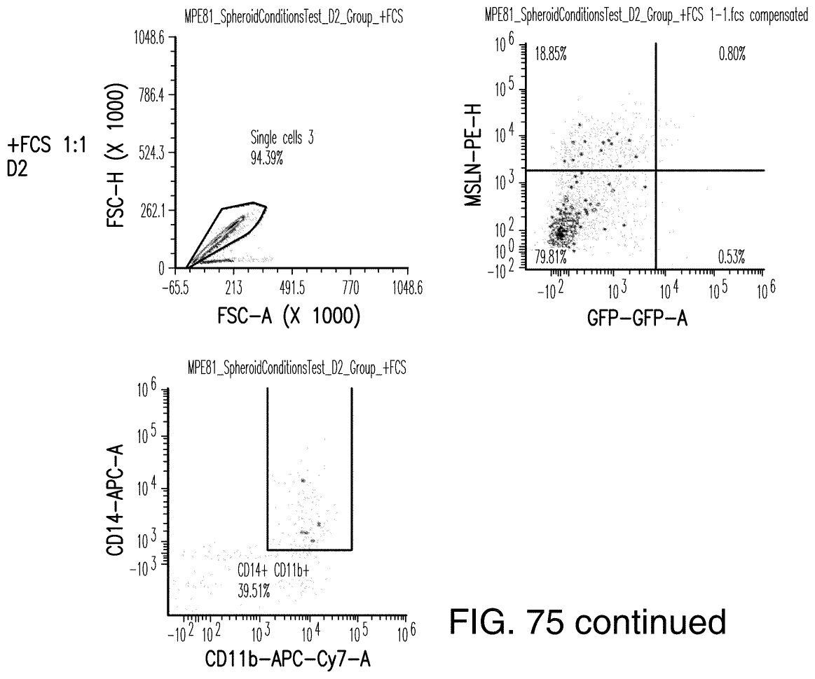

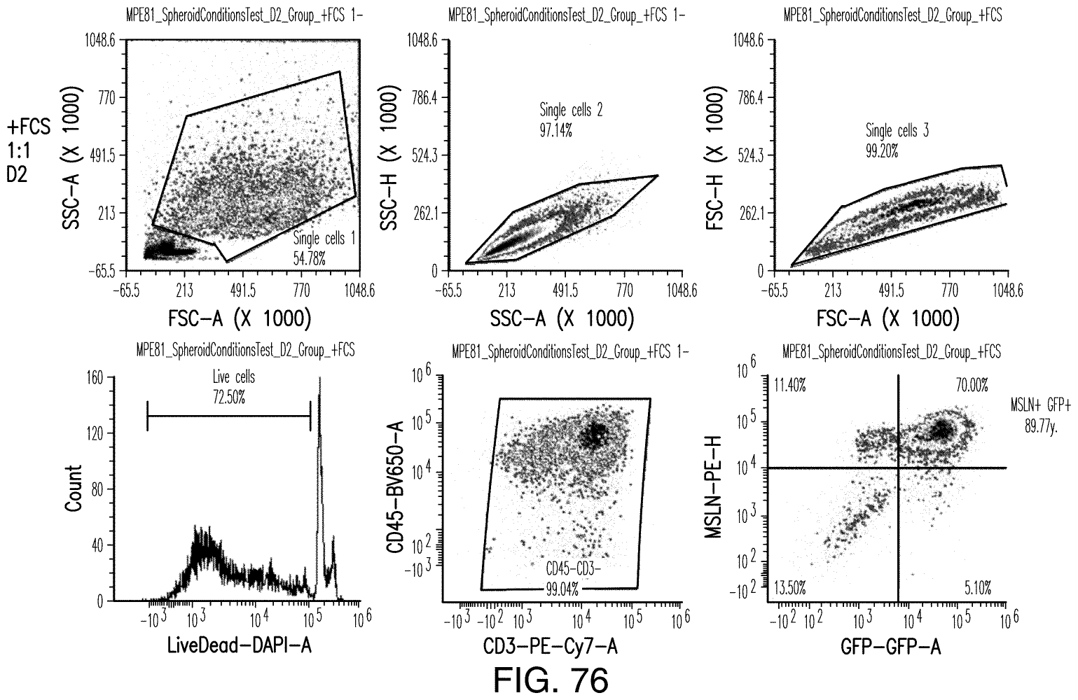

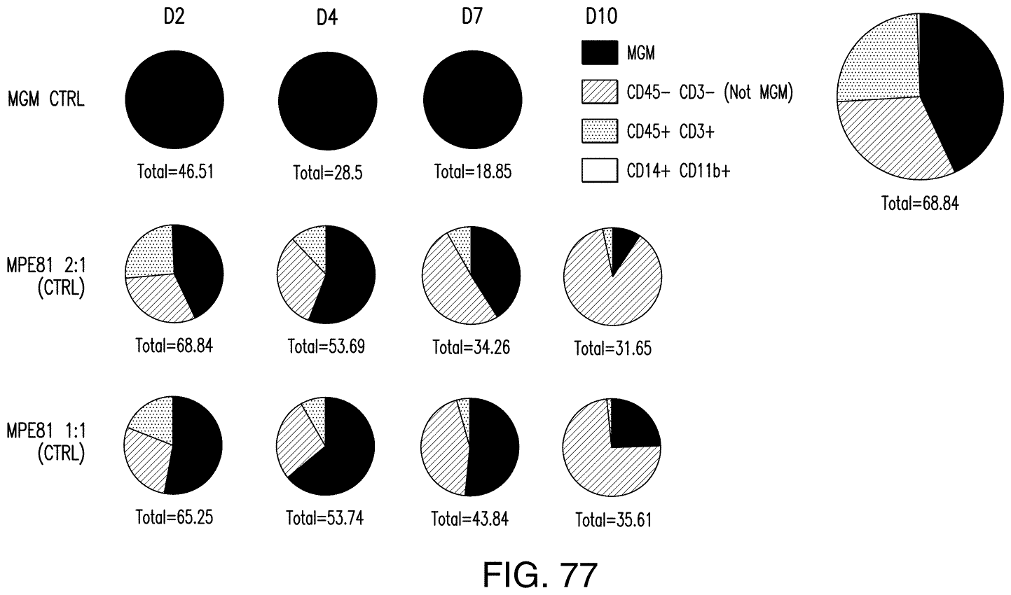

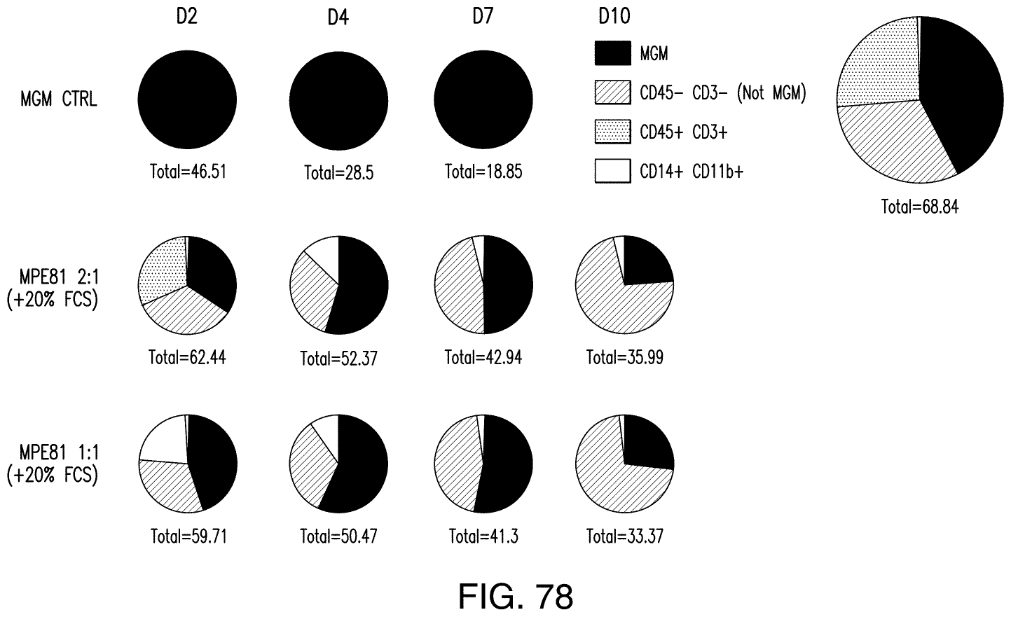

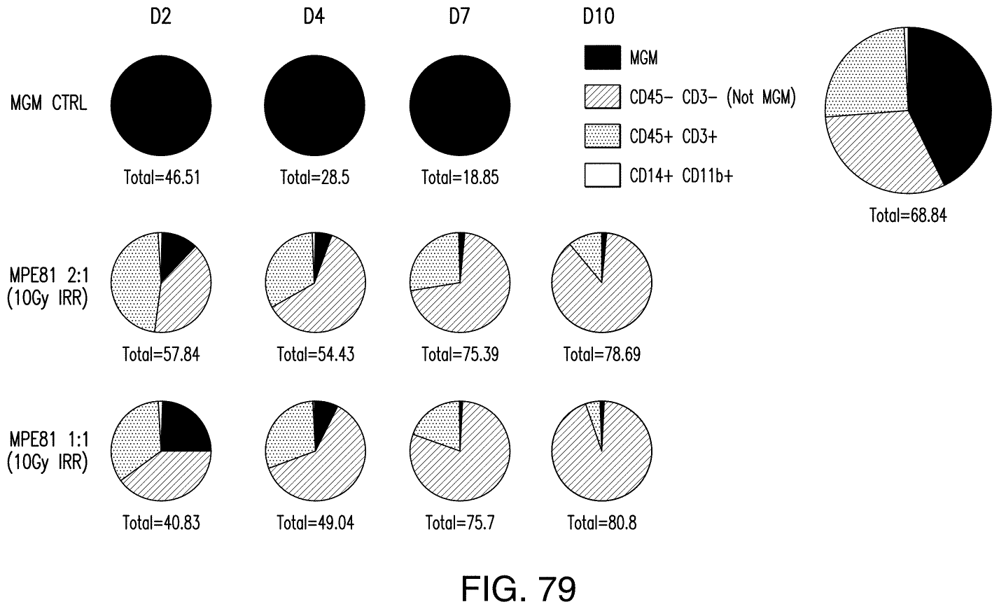

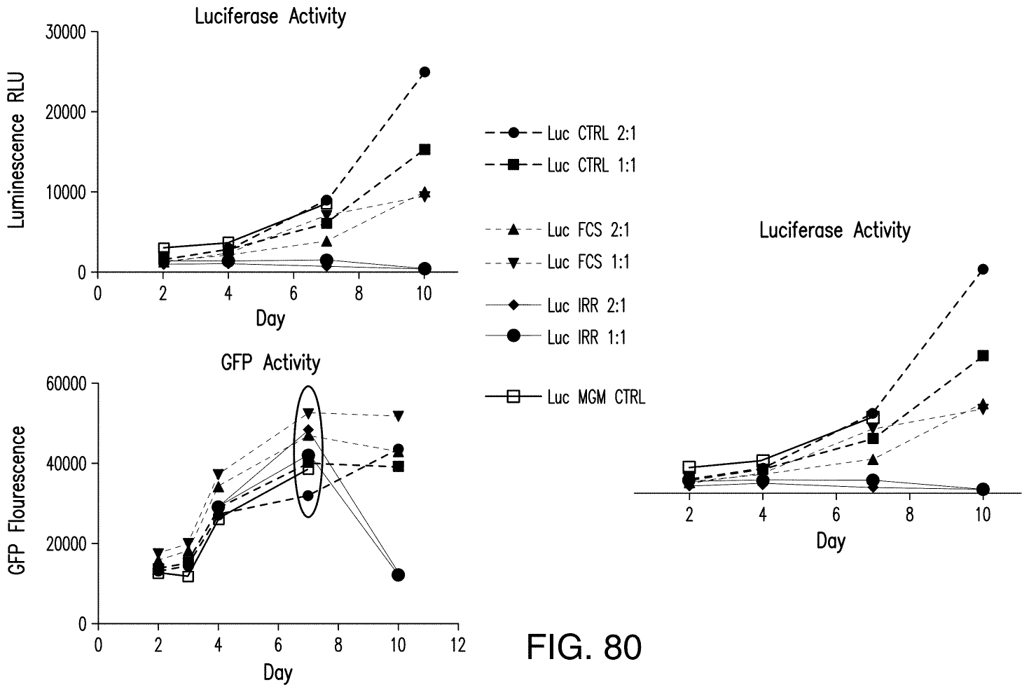

ZEN microscopic brightfield and GFP merged images of the cells were shown (FIG. 74). The flow cytometry gating strategy for cell populations at D2 was presented for immune cells (FIG. 75) and MGM tumor cells (FIG. 76). Cell populations CD45+CD3+ and CD14+CD11b+ of the cells with or without doses of radiation or with different concentrations of FCS were assessed using flow cytometry at day 2, day 4, day 7 and day 10 (FIGS. 77-79). MGM tumor cell population were analyzed using GFP and luciferin activity assay (FIG. 80).

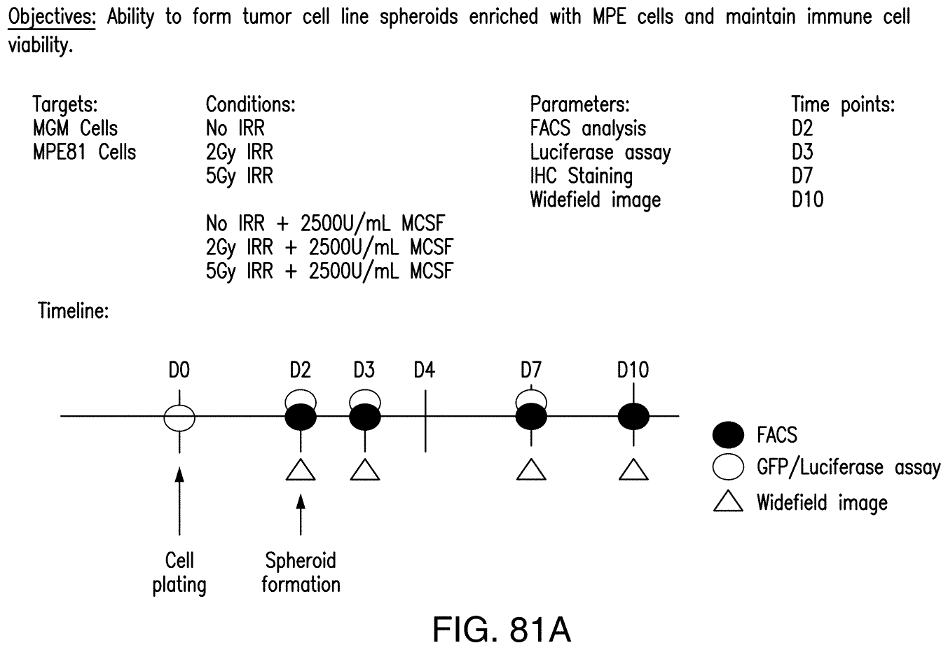

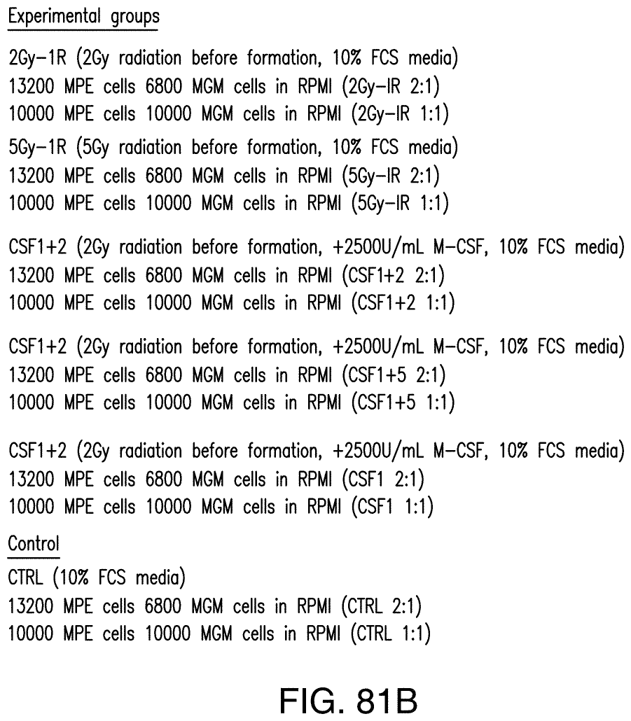

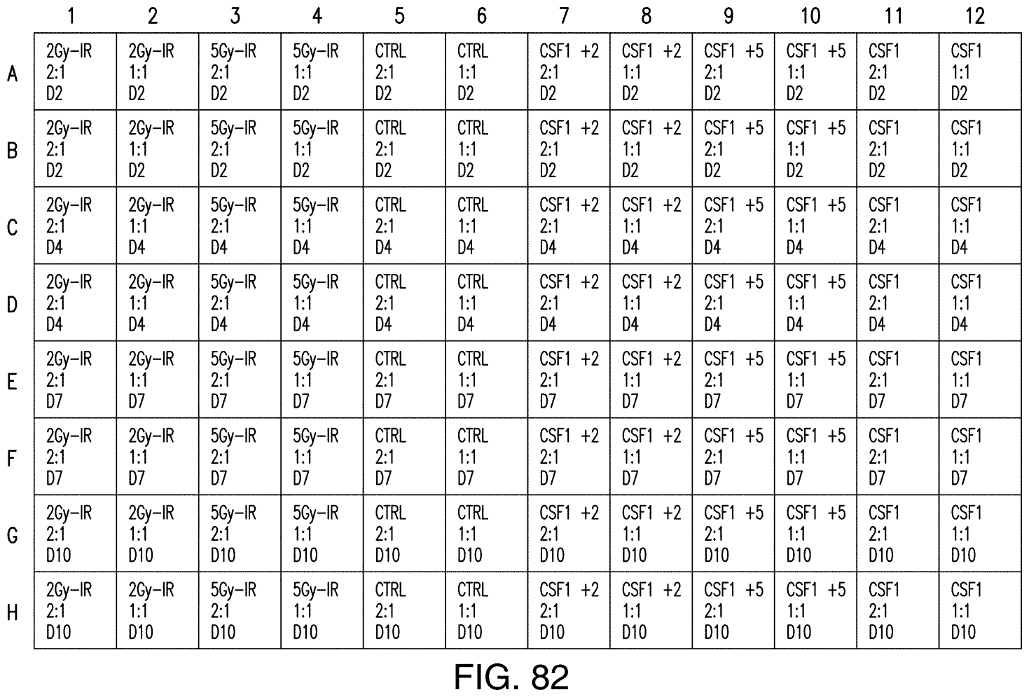

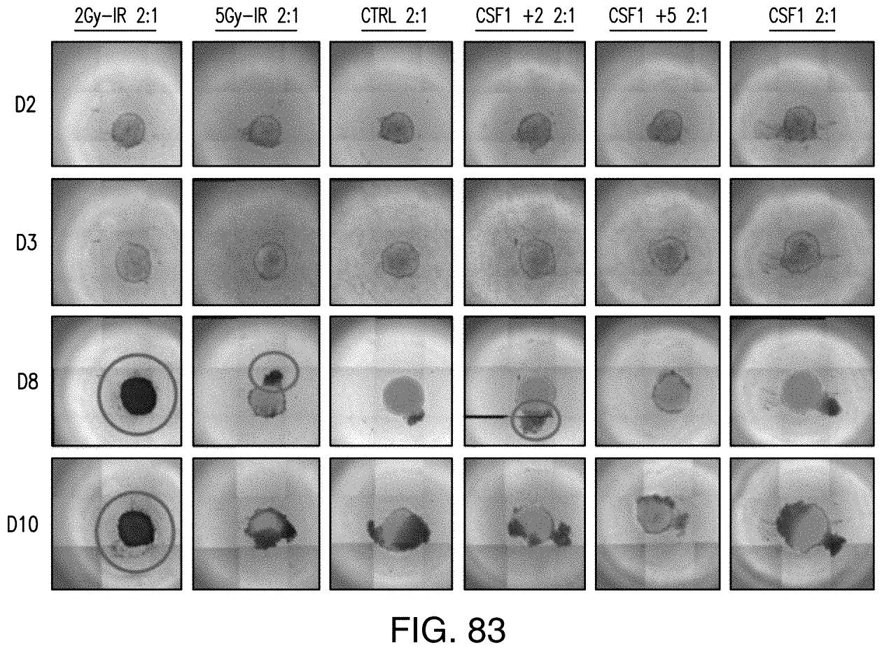

Experimental scheme to identify the ability to form MGM tumor cell line spheroids enriched with MPE cells and maintain immune cell viability was laid out. Cells were incubated with or without different doses of radiation or with or without addition of CSF1 factor at different concentrations. CTRL (control), 2 Gy IR, 5Gy IR, 2Gy IR±CSF, 5Gy IR±CSF. MPE cells and MGM cells were mixed at the ratio of 2:1 or 1:1 in RPMI cell culture medium, and were plated in a 96 well tissue culture plate. Cells were analyzed using ZEN brightfield and GFP merged images over time microscopic (FIGS. 81-83).

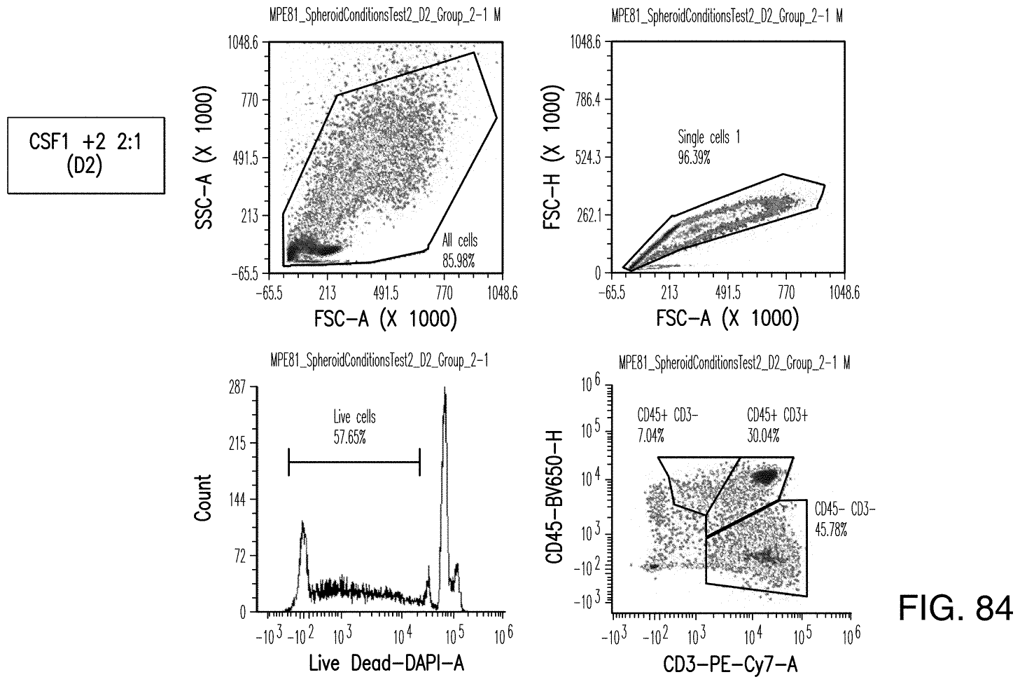

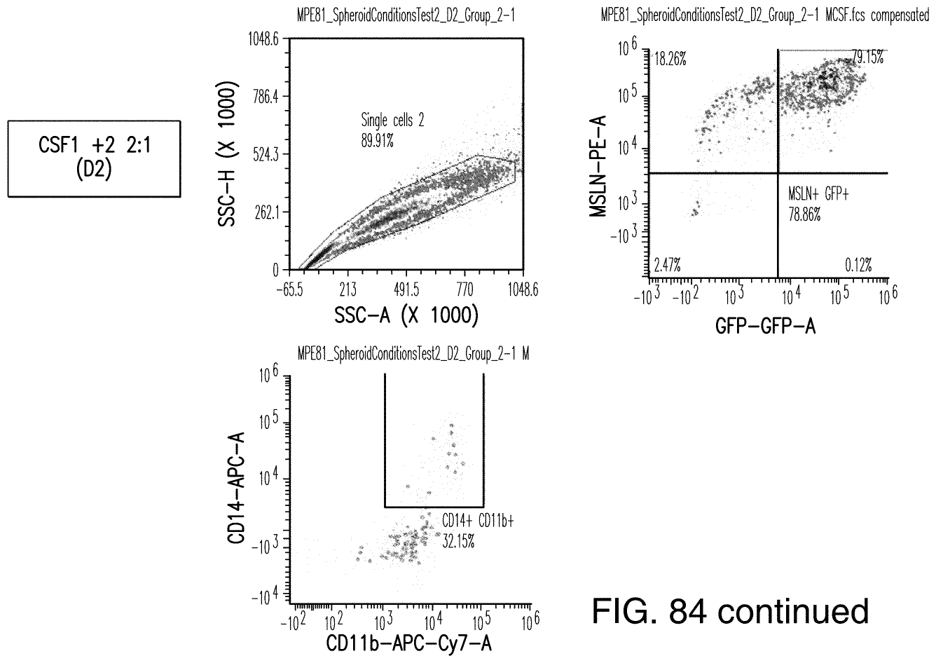

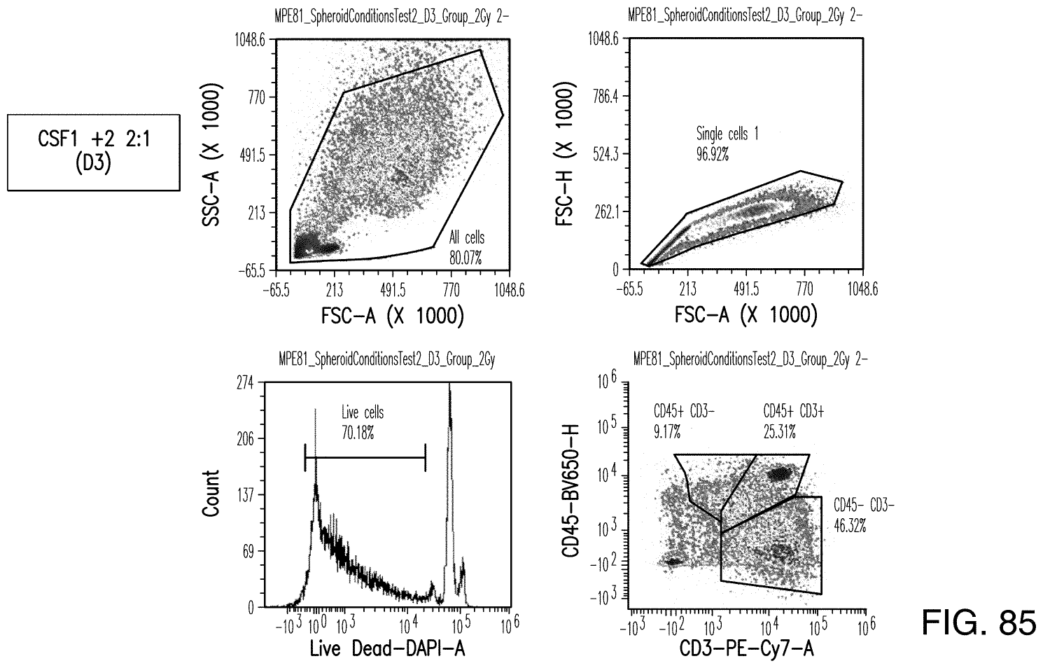

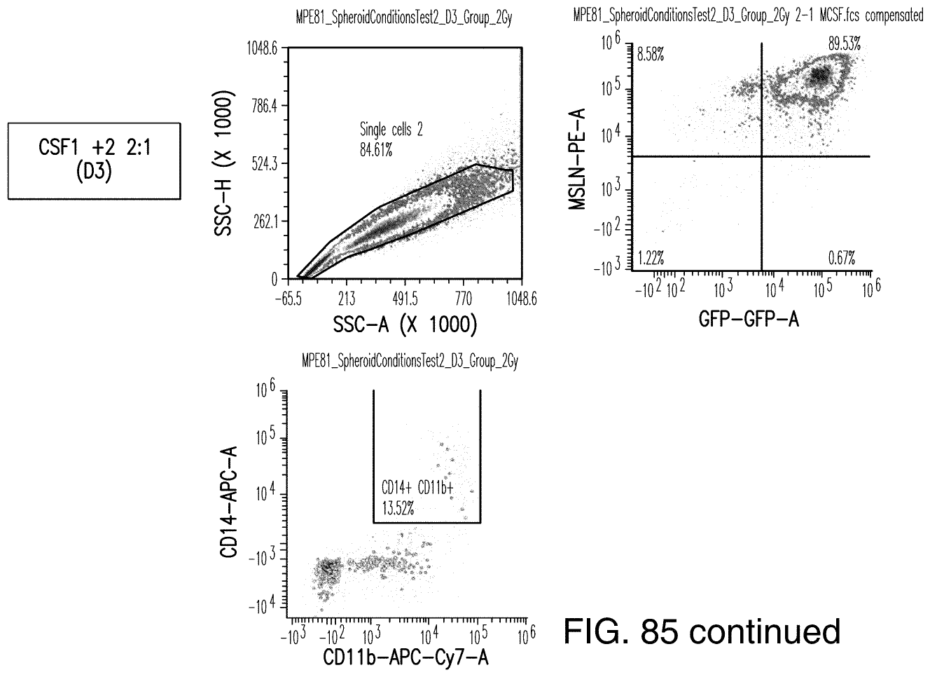

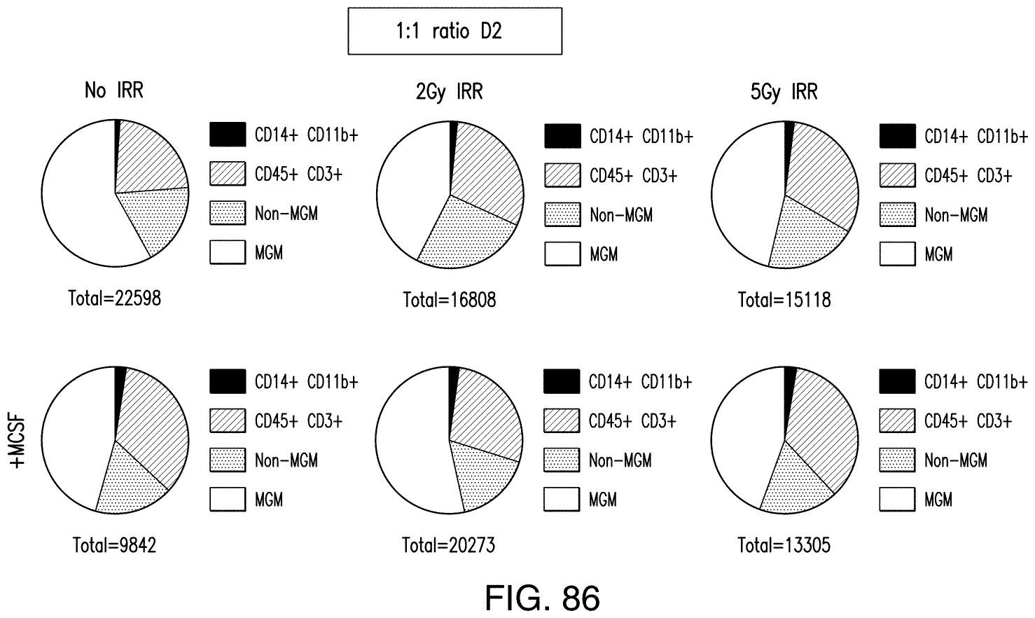

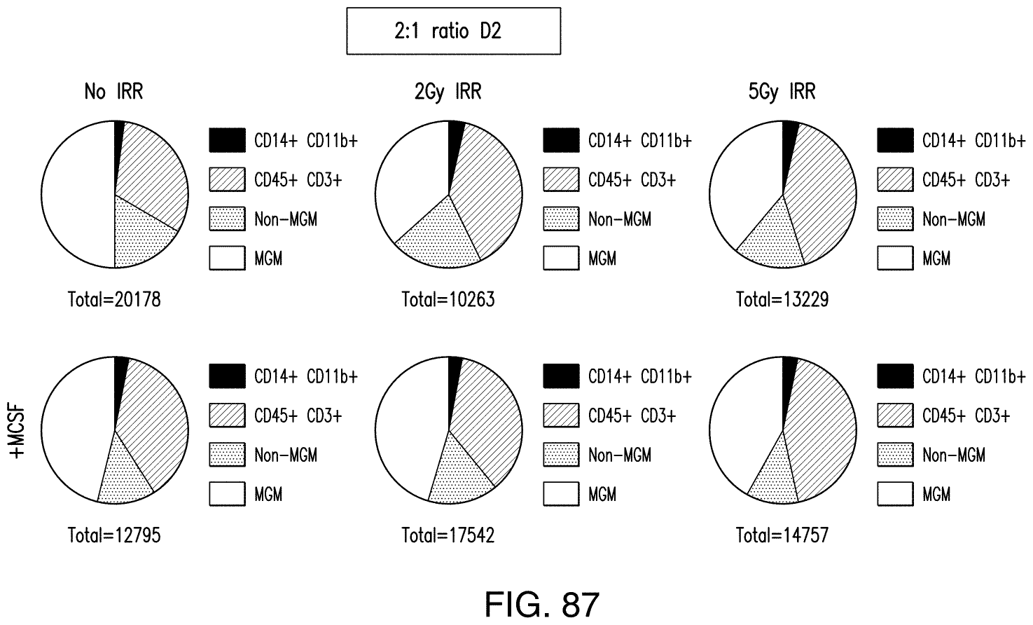

The effects of ionizing radiations on cell populations for CD45+CD3+ and CD14+CD11b+ were analyzed using flow cytometry at day 2 (D2) and 3 (D3) (FIGS. 84-85). The effects of ionizing radiations on cell populations for CD45+CD3+ and CD14+CD11b+ were analyzed using flow cytometry at day 2 (D2) and 3 (D3). The experimental groups consist of cells with or without different doses of radiation or with or without addition of CSF1 factor at different concentrations (FIGS. 86-87).

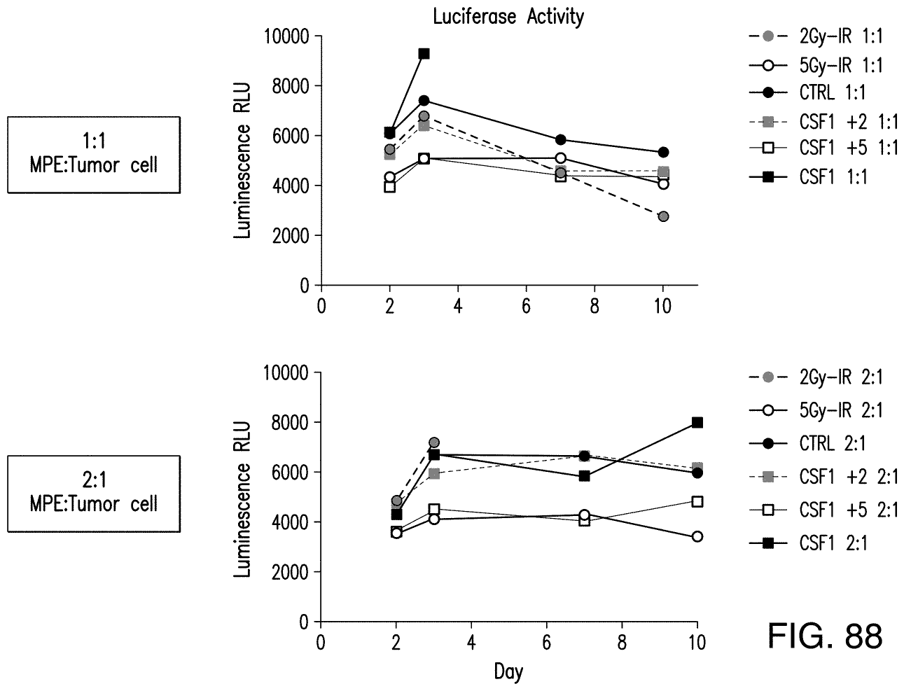

MGM tumor cell population were assessed using luciferase activity assay (FIG. 88). Advantages of 3D models include: 3D models more accurately mimic tissue-like structures better than 2D cell cultures; 3D models can exhibit differentiated cellular function not present in 2D cell cultures; and some findings demonstrate that 3D models may be more predictive of in vivo response to drug treatments (Weiswald et al., 2015, Neoplasia).

The objective of the experiment is to explore the ability of tumor cell lines to form spheroids enriched with MPE cells. The present example shows that it was possible to form multicellular tumor spheroids (MCTS) that incorporated immune cell populations from malignant pleural effusion (MPE). Formation of MCTS at a given MPE:Tumor Cell ratio was dependent on the MPE. Immune cell populations were depleted in current culture conditions. Suggested from luciferase activity and FACS data, tumor spheroid viability began to decrease between day 7 and day 10. The present example further showed that additional FCS did not appear to favor immune cells over MGM cells. 10Gy irradiation was too strong and resulted in significant MGM cell death even after 2 days.

Although the presently disclosed subject matter and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the inventions of which are incorporated herein by reference in their entireties for all purposes.

What is claimed is:

1. A method for assessing cytotoxicity of an immunoresponsive cell, comprising:
(a) culturing an immunoresponsive cell in a pleural effusion;
(b) contacting the immunoresponsive cell with a target cell; and
(c) measuring a status of the target cell contacted with the immunoresponsive cell,
wherein the status of the target cell indicates the cytotoxicity of the immunoresponsive cell.

2. The method of claim 1, wherein (b) comprises culturing the target cell and immunoresponsive cell in the pleural effusion.

3. A method for assessing the effect of an immunotherapeutic agent on cytotoxicity of an immunoresponsive cell, comprising
(a) culturing an immunoresponsive cell in a first pleural effusion;
(b) contacting the immunoresponsive cell with a target cell;
(c) measuring a status of the target cell contacted with the immunoresponsive cell, wherein the status indicates the cytotoxicity of the immunoresponsive cell;
(d) culturing the immunoresponsive cell in a second pleural effusion;
(e) contacting an immunotherapeutic agent with the immunoresponsive cell;
(f) contacting the immunoresponsive cell with the target cell;
(g) measuring the status of the target cell contacted with the immunoresponsive cell; and
(h) comparing the status measured in (g) with the status measured in (c), wherein a change between the status measured in (g) and the status measured in (c) indicates that the immunotherapeutic agent has an effect on cytotoxicity of the immunoresponsive cell.

4. The method of claim 3, wherein (b) comprises culturing the target cell and immunoresponsive cell in the first pleural effusion; (0 comprises culturing the target cell and immunoresponsive cell in the second pleural effusion; and/or (e) comprises culturing the immunotherapeutic agent and the immunoresponsive cell in the second pleural effusion.

5. The method of claim 1, wherein the target cell comprises a tumor antigen or a pathogen antigen.

6. The method of claim 1, wherein the status of the target cell is selected from the group consisting of cell death, cell proliferation, cell apoptosis, cell necrosis, cell autophagy, cell lysis, cell growth arrest, cell antigen expression suppression, cell chemokine receptor expression, cell chemokine secretion, cell receptor expression, cell ligand expression and combinations thereof.

7. The method of claim 1, wherein the status of the target cell is measured by a Cr⁵¹ release assay, a bioluminescence assay, a flow cytometry assay, an impedance assay, an apoptosis assay, an assay measuring chemokine secretion, an assay measuring cell ligand expression, an assay measuring cell receptor expression, or a combination of the foregoing.

8. The method of claim 1, wherein the pleural effusion is obtained from a subject, or two or more subjects.

9. The method of claim 8, wherein the subject suffers from cancer.

10. The method of claim 1, where the pleural effusion is obtained from a subject who previously received an anti-cancer agent.

11. The method of claim 10, wherein the anti-cancer agent is selected from the group consisting of an immune checkpoint inhibitor, cytokines, oncolytic virus, T cells, dendritic cells, bispecific antibodies, BiTEs, immunotoxins, and combinations thereof.

12. The method of claim 11, wherein the pleural effusion is substantially free of immune cells, or comprises immune cells.

13. The method of claim 12, wherein the immune cells are selected from the group consisting of T cells, B cells, Nature Killer (NK) cells, neutrophils, macrophages, dendritic cells, cytotoxic T lymphocytes (CTLs), regulatory T cells (Tregs), Natural Killer T (NKT) cells, and combinations thereof.

14. The method of claim 1, wherein the pleural effusion has an immunosuppressive effect.

15. The method of claim 1, wherein the method comprises culturing the immunoresponsive cells in the pleural effusion for at least about 30 minutes, up to about 72 hours, and/or about 24 hours before its initial contact with the target cell.

16. The method of claim 1, wherein the method comprises measuring the status of the target cell at least about 1 hour, no later than about 72 hours, and/or about 18 hours from the initial contact of the immunoresponsive cell with the target cell.

17. The method of claim 1, wherein the immunoresponsive cell comprises a chimeric antigen receptor (CAR) that binds to an antigen, and the CAR comprises an extracellular antigen-binding domain, a transmembrane domain, and an intracellular signaling domain.

18. The method of claim 17, wherein the extracellular antigen-binding domain specifically binds to mesothelin; the transmembrane domain comprises a CD28 polypeptide; the intracellular signaling domain comprises a CD3 polypeptide; and/or the intracellular signaling domain further comprises a co-stimulatory signaling domain, wherein the co-stimulatory signaling region comprises a CD28 polypeptide.

19. The method of claim 1, wherein the immunoresponsive cell is selected from the group consisting of T cells, Natural Killer (NK) cells, human embryonic stem cells, and pluripotent stem cells from which lymphoid cells may be differentiated.

20. The method of claim 19, wherein the immunoresponsive cell is a T cell.

21. The method of claim 20, wherein the T cell is selected from the group consisting of cytotoxic T lymphocytes (CTLs), regulatory T cells (Tregs), and Natural Killer T (NKT) cells.

22. A kit for assessing cytotoxicity of an immunoresponsive cell, comprising a pleural effusion, an immunoresponsive cell, and a target cell.

23. A kit for assessing the effect of an immunotherapeutic agent on cytotoxicity of an immunoresponsive cell, comprising: a pleural effusion, an immunoresponsive cell, and a target cell.

24. A system for assessing cytotoxicity of an immunoresponsive cell, comprising a pleural effusion, an immunoresponsive cell, and a target cell.

25. A system for assessing the effect of an immunotherapeutic agent on cytotoxicity of an immunoresponsive cell, comprising: a pleural effusion, an immunoresponsive cell, and a target cell.


SEQUENCE LISTING (19 sequences)

SEQ ID NO: 1 - protein, 10 aa, Artificial Sequence
  Description of Artificial Sequence Synthetic peptide
GGSVSSGSYY

SEQ ID NO: 2 - protein, 7 aa, Artificial Sequence
  Description of Artificial Sequence Synthetic peptide
IYYSGST

SEQ ID NO: 3 - protein, 12 aa, Artificial Sequence
  Description of Artificial Sequence Synthetic peptide
AREGKNGAFDIW

SEQ ID NO: 4 - protein, 6 aa, Artificial Sequence
  Description of Artificial Sequence Synthetic peptide
QSISSY

SEQ ID NO: 5 - protein, 4 aa, Artificial Sequence
  Description of Artificial Sequence Synthetic peptide
AASS

SEQ ID NO: 6 - protein, 10 aa, Artificial Sequence
  Description of Artificial Sequence Synthetic peptide
QQSYSTPLTF

SEQ ID NO: 7 - protein, 119 aa, Artificial Sequence
  Description of Artificial Sequence Synthetic polypeptide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSS

SEQ ID NO: 8 - protein, 109 aa, Artificial Sequence
  Description of Artificial Sequence Synthetic polypeptide
RHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRT

SEQ ID NO: 9 - protein, 15 aa, Artificial Sequence
  Description of Artificial Sequence Synthetic peptide
GGGGSGGGGSGGGGS

SEQ ID NO: 10 - DNA, 357 bp, Artificial Sequence
  Description of Artificial Sequence Synthetic polynucleotide
caggttcagcttcaggagagtggcccaggcctggtgaagccaagtgagactctcagcttgacttgcacagtttctggaggcagtgtctcctcaggcagctattattggtcctggattcggcagccccctgggaaaggcctggagtggattgggtacatatattacagtggcagcacaaattacaatccatccctgaagtctcgagtaactatcagtgtggacacaagcaagaatcagttttcactcaaactgtcttctgtgactgctgctgacactgctgtttattattgtgccagggaggggaaaaatggggcatttgatatttggggtcagggcacaatggtgacagtcagctct

SEQ ID NO: 11 - DNA, 327 bp, Artificial Sequence
  Description of Artificial Sequence Synthetic polynucleotide
cgccatcagatgactcagtccccctccagtctttctgcctcagttggggatagagtgaccatcacatgcagagcaagtcagagcatatcatcctatctgaactggtaccagcagaagccagggaaagcccccaaattgctgatttatgcagcctcaagtctccagagtggggtgccaagcaggttctcaggcagtggcagtgggacagatttcacattgacaatcagctccctccaacctgaagattttgccacctactattgccagcaatcctacagcacgcccctgacttttggaggtggcacaaaggtagagatcaagaggact

SEQ ID NO: 12 - protein, 164 aa, Unknown
  Description of Unknown CD3-zeta sequence
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - DNA, 339 bp, Unknown
  Description of Unknown CD3-zeta sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 14 - protein, 41 aa, Unknown
  Description of Unknown CD28 sequence
RSKRSRLLHSDYMNMTPRRPGPTRKHYQPYAPPRDFAAYRS

SEQ ID NO: 15 - DNA, 123 bp, Unknown
  Description of Unknown CD28 sequence
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 16 - protein, 27 aa, Homo sapiens
FWVLVVVGGVLACYSLLVTVAFIIFWV

SEQ ID NO: 17 - DNA, 81 bp, Homo sapiens
ttttgggtgctggtggtggttggtggagtcctggcttgctatagcttgctagtaacagtggcctttattattttctgggtg

SEQ ID NO: 18 - protein, 6 aa, Artificial Sequence
  Description of Artificial Sequence Synthetic peptide
IEGRMD

SEQ ID NO: 19 - protein, 7 aa, Artificial Sequence
  Description of Artificial Sequence Synthetic peptide
DIEGRMD